(19) United States
(12) Patent Application Publication
(10) Pub. No.: US 2022/0344002 A1
(43) Pub. Date: Oct. 27, 2022

(54) SOLUBLE MEDIATORS FOR PREDICTING SYSTEMIC LUPUS ERYTHEMATOSUS ACTIVITY EVENTS

(71) Applicant: Oklahoma Medical Research Foundation, Oaklahoma City, OK (US)

(72) Inventors: Judith Ann JAMES, Edmond, OK (US); Melissa E. MUNROE, Edmond, OK (US)

(21) Appl. No.: 17/762,281

(22) PCT Filed: Sep. 18, 2020
(86) PCT No.: PCT/US20/51482

§ 371 Date: Mar. 21, 2022

Related U.S. Application Data
(60) Provisional application No. 62/903,551, filed on Sep. 20, 2019.

(51) Int. Cl. G16B 25/10; G01N 33/564; C12Q 1/6883; G16B 40/20; G16H 10/40

(52) U.S. Cl. CPC G16B 25/10; G01N 33/564; C12Q 1/6883; G16B 40/20; G16H 10/40; C12Q 2600/158; G01N 2800/104; G01N 2800/54; G01N 2800/60

(57) ABSTRACT

Systemic Lupus Erythematosus is marked by altered immune regulation linked to waxing and waning clinical disease. Embodiments described herein identify sets of biomarkers/mediators and their use for informing and/or predicting a future SLE disease activity event such as an impending SLE flare or SLE-related organ inflammation. Such an approach can be beneficial in the management of lupus.

### CROSS REFERENCE TO RELATED APPLICATIONS

This application claims the benefit of U.S. Provisional Application 62/903,551 filed 20 Sep. 2019, the content of which is incorporates herein by reference.

### STATEMENT REGARDING FEDERALLY SPONSORED RESEARCH OR DEVELOPMENT

This invention was made with government support under award numbers: 5U19AI082714-10, 5P30GM103510-05, 5U54GM104938-07, 3P30AR053483-10, and 1P30AR073750-01 awarded by the National Institute of Allergy, Immunology and Infectious Diseases; National Institute of Arthritis, Musculoskeletal and Skin Disease, and National Institute of General Medical Sciences. The government has certain rights in the invention.

### BACKGROUND OF THE INVENTION

Systemic lupus erythematosus (SLE) is a chronic, debilitating autoimmune disease that causes irreversible organ damage, contributing to diminished quality of life and early mortality (1, 2). Most SLE patients experience periods of relatively quiescent disease punctuated with periods of increased clinical activity (3). Because clinical disease flares and the major immunosuppressants used to subdue disease activity can both cause irreparable damage (1), the frequency and severity of flares are important prognostic indicators for long term SLE outcomes (4-6). In patients receiving standard-of-care treatments, rates of flare range from 0.24 to 1.8 flares per person-year (5-7). Treatment typically relies on rapidly acting, toxic agents such as steroids. Earlier identification and treatment of flares might prevent significant organ damage and improve the quality of life for patients with SLE (8).

This would be particularly useful in African American (AA) SLE patients, who frequently experience a more aggressive disease course. AA SLE patients face an increased risk of developing irreversible organ system involvement, including permanent CNS, pulmonary, and cardiovascular damage (9-12), lupus nephritis and end-stage renal disease (13), and a three-fold increase in SLE-related mortality compared to European American (EA) patients (14).

Current approaches for forecasting clinical disease flares have some utility but remain inadequate, as evidenced by the studies summarized below. For example, prior efforts have focused on a single pathway (e.g. Type I IFN), which has resulted in the capture of an inaccurate number of patients that are in a pre-flare state (28). SLE is known to be very heterogenous (29): therefore, capturing the heterogeneity for the purposes of predicting an upcoming SLE disease activity event (such as a flare or new/worsening organ damage) has been difficult.

### SUMMARY

Embodiments described herein use a predictive model to analyze quantitative expression levels of a set of soluble mediators. Such a predictive model generates a score representing a likelihood of a SLE disease activity event, such as an impending clinical disease flare or future manifestation of organ inflammation and damage. As described in the different embodiments below, the set of soluble mediators analyzed by the predictive model can include different numbers of soluble mediators (e.g., 9 biomarkers, 10 biomarkers, 14 biomarkers, 31 biomarkers, and other combinations). Each of these combinations of soluble mediators is informative for assessing the likelihood of a SLE disease activity event.

Disclosed herein is a method comprising: (a) obtaining a dataset comprising expression levels of biomarkers from a test sample from a systemic lupus erythematosus (SLE) subject, wherein the biomarkers comprise: at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; (b) generating a Lupus Flare Predictive Index (LFPI) based on the expression levels in the obtained dataset; and (c) determining likelihood of a future SLE disease activity event in the SLE subject based on the LFPI. In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ). In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1); the at least two TNFR superfamily member molecules comprise tumor necrosis factor receptor I (TNFRI), tumor necrosis factor receptor II (TNFRII), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

Additionally disclosed herein is a method comprising: (a) obtaining a dataset comprising expression levels of biomarkers from a test sample from a systemic lupus erythematosus (SLE) subject, wherein the biomarkers comprise: at least one innate cytokine selected from IL-7, IL-1α, and IL-1β; at least one Th1 cytokine selected from interferon-gamma (IFN-γ), IL-12p70, IL-2, and IL-2R_{α}, at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α). C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; (b) generating a LFPI based on the expression levels in the obtained dataset; and (c) determining likelihood of a future SLE disease activity event in the SLE subject based on the LFPI.

Additionally disclosed herein is a method comprising: (a) obtaining or having obtained a dataset comprising expression levels of biomarkers from a test sample from a systemic lupus erythematosus (SLE) subject, wherein the biomarkers comprise: (i) chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); (ii) tumor necrosis factor receptor (TNFR) superfamily member molecules, wherein the TNFR superfamily member molecules comprise: a tumor necrosis factor receptor I (TNFRI), and a tumor necrosis factor receptor II (TNFRII); (iii) regulatory mediator molecules, wherein the regulatory mediator molecules comprise: native transforming growth factor beta (native TGF-β), and an interleukin-1 receptor antagonist (IL-1RA); and (iv) one or more systemic lupus erythematosus (SLE) mediator molecules, wherein the one or more SLE mediator molecules comprise a stem cell factor (SCF); (b) generating a LFPI based on the expression levels in the obtained dataset; and (c) determining likelihood of a future SLE disease activity event in the SLE subject based on the LFPI. In some embodiments, the biomarkers further comprise one or more T-helper type-1 (Th1) cytokines, wherein the one or more Th1 cytokines comprise an interferon-gamma (IFN-γ). In some embodiments, the tumor necrosis factor receptor (TNFR) superfamily member molecules further comprise a tumor necrosis factor-related apoptosis-inducing ligand (TRAIL). In some embodiments, the chemokine(s) or adhesion molecules further comprise: a C—C motif chemokine ligand 4 (CCL4)/macrophage inflammatory protein-1 beta (MIP-1β) and an Intercellular Adhesion Molecule 1 (ICAM-1). In some embodiments, the regulatory mediator molecules further comprise total TGF-β. In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1); the TNFR superfamily member molecules further comprise a tumor necrosis factor-related apoptosis-inducing ligand (TRAIL); and the regulatory mediator molecules further comprise a total transforming growth factor beta (total TGF-β). In some embodiments, the biomarkers further comprise (vi) one or more innate cytokines, wherein the one or more innate cytokines are selected from IL-7, IL-1α, and IL-1 β. In some embodiments, the biomarkers further comprise (vii) one or more T-helper type-2 (Th2) cytokines, wherein the one or more Th2 cytokines are selected from IL-13 and IL-4. In some embodiments, the biomarkers further comprise (viii) one or more Th17 cytokines, wherein the one or more Th17 cytokines comprise IL-17A. In some embodiments, the one or more SLE mediator molecules further comprise Resistin. In some embodiments, the Th1 cytokines further comprise IL-2, IL-12p70, and IL-2R_{α}. In some embodiments, the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

In some embodiments, the Th1 cytokines comprise interferon-gamma (IFN-γ), IL-2R_{α}, IL-12p70, and IL-2; the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8; the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α; the regulatory mediator molecules comprise: native transforming growth factor beta (native TGF-β), an interleukin-1 receptor antagonist (IL-1RA), a total transforming growth factor beta (total TGF-β), and IL-10; the SLE mediator molecules comprise: a stem cell factor (SCF) and Resistin; and wherein the biomarkers further comprise: innate cytokines, wherein the innate cytokines comprise: IL-7, IL-1α, and IL-1β; T-helper type-2 (Th2) cytokines, wherein the Th2 cytokines comprise: IL-13 and IL-4; and one or more Th17 cytokines, wherein the one or more Th17 cytokines comprise IL-17A.

In some embodiments, the expression levels of biomarkers comprise protein levels. In some embodiments, the expression levels of biomarkers are determined using one of an ELISA assay, xMAP® technology, or SimplePlex™ assay. In some embodiments, the expression levels of biomarkers comprise mRNA levels. In some embodiments, the mRNA levels are obtained from circulating cells. In some embodiments, the mRNA levels are obtained from circulating T-cells.

In some embodiments, generating the LFPI based on the expression levels comprises applying a predictive model. In some embodiments, applying the predictive model comprises, for the expression level of each biomarker: log-transforming the expression level; standardizing the expression level: obtaining a corresponding coefficient for the biomarker; the corresponding coefficient representing an association between pre-flare expression levels of the biomarker and a measurement of SLE clinical disease activity; and weighting the standardized expression level with the corresponding coefficient to obtain a LFPI subscores for the biomarker; and summing the LFPI subscores to obtain the LFPI. In some embodiments, for each expression level, the corresponding coefficient is obtained from a linear regression model testing associations between the measurement of SLE clinical disease activity and the pre-flare expression levels of the biomarker.

In some embodiments, standardizing the expression level comprises normalizing the expression level to a mean expression value for SLE patients with stable SLE disease. In some embodiments, the biomarkers were selected for inclusion in the dataset using an applied machine learning modeling approach. In some embodiments, the applied machine learning modeling approach is one of random forest or gradient boosting. In some embodiments, the measurement of SLE clinical disease activity is the Safety of Estrogens in Lupus Erythematosus National Assessment-Systemic Lupus Erythematosus Disease Activity Index (SELENA-SLEDAI). In some embodiments, the measurement of SLE clinical disease activity is determined from samples obtained from a group of patients undergoing a flare event. In some embodiments, performance of the predictive model is characterized by an area under a receiver operating characteristic curve that is greater than 0.85. In some embodiments, performance of the predictive model is characterized by an area under a receiver operating characteristic curve that is greater than 0.90.

In some embodiments, the method further comprises administering a treatment to the SLE subject. In some embodiments, obtaining the dataset comprising expression levels of biomarkers comprises: obtaining a blood, serum or plasma sample from the SLE subject; and assessing expression levels of biomarkers from the test sample from the SLE subject. In some embodiments, the future SLE disease activity event is one of a future flare event or future organ damage. In some embodiments, the dataset further comprises expression levels of biomarkers from a second test sample taken from the systemic lupus erythematosus (SLE) subject at a different time point.

Additionally disclosed herein is a non-transitory computer readable medium storing instructions that, when executed by a processor, cause the processor to perform the steps of: (a) obtaining a dataset comprising expression levels of biomarkers from a test sample from a systemic lupus erythematosus (SLE) subject, wherein the biomarkers comprise: at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; (b) generating a LFPI based on the expression levels in the obtained dataset; and (c) determining likelihood of a future SLE disease activity event in the SLE subject based on the LFPI. In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ). In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1); the at least two TNFR superfamily member molecules comprise tumor necrosis factor receptor I (TNFRI), tumor necrosis factor receptor II (TNFRII), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

Additionally disclosed herein is a non-transitory computer readable medium storing instructions that, when executed by a processor, cause the processor to perform the steps of: (a) obtaining a dataset comprising expression levels of biomarkers from a test sample from a systemic lupus erythematosus (SLE) subject, wherein the biomarkers comprise: at least one innate cytokine selected from IL-7, IL-1α, and IL-1β; at least one Th1 cytokine selected from interferon-gamma (IFN-γ), IL-12p70, IL-2, and IL-2R_{α}; at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; (b) generating a LFPI based on the expression levels in the obtained dataset; and (c) determining likelihood of a future SLE disease activity event in the SLE subject based on the LFPI.

Additionally disclosed herein is a non-transitory computer readable medium storing instructions that, when executed by a processor, cause the processor to perform the steps of: (a) obtaining a dataset comprising expression levels of biomarkers from a test sample from a systemic lupus erythematosus (SLE) subject, wherein the biomarkers comprise: (i) chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); (ii) tumor necrosis factor receptor (TNFR) superfamily member molecules, wherein the TNFR superfamily member molecules comprise: a tumor necrosis factor receptor I (TNFRI), and a tumor necrosis factor receptor II (TNFRII); (iii) regulatory mediator molecules, wherein the regulatory mediator molecules comprise: native transforming growth factor beta (native TGF-β), and an interleukin-1 receptor antagonist (IL-1RA); and (iv) one or more systemic lupus erythematosus (SLE) mediator molecules, wherein the one or more SLE mediator molecules comprise a stem cell factor (SCF); (b) generating a LFPI based on the expression levels in the obtained dataset; and (c) determining likelihood of a future SLE disease activity event in the SLE subject based on the LFPI. In some embodiments, the biomarkers further comprise one or more T-helper type-1 (Th1) cytokines, wherein the one or more Th1 cytokines comprise an interferon-gamma (IFN-γ). In some embodiments, the tumor necrosis factor receptor (TNFR) superfamily member molecules further comprise a tumor necrosis factor-related apoptosis-inducing ligand (TRAIL). In some embodiments, the chemokine(s) or adhesion molecules further comprise: a C—C motif chemokine ligand 4 (CCL4)/macrophage inflammatory protein-1 beta (MIP-1β) and an Intercellular Adhesion Molecule 1 (ICAM-1). In some embodiments, the regulatory mediator molecules further comprise total TGF-β. In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1); the TNFR superfamily member molecules further comprise a tumor necrosis factor-related apoptosis-inducing ligand (TRAIL); and the regulatory mediator molecules further comprise a total transforming growth factor beta (total TGF-β). In some embodiments, the biomarkers further comprise (vi) one or more innate cytokines, wherein the one or more innate cytokines are selected from IL-7, IL-1α, and IL-1β. In some embodiments, the biomarkers further comprise (vii) one or more T-helper type-2 (Th2) cytokines, wherein the one or more Th2 cytokines are selected from IL-13 and IL-4. In some embodiments, the biomarkers further comprise (viii) one or more Th17 cytokines, wherein the one or more Th17 cytokines comprise IL-17A. In some embodiments, the one or more SLE mediator molecules further comprise Resistin. In some embodiments, the Th1 cytokines further comprise IL-2, IL-12p70, and IL-2R_{α}. In some embodiments, the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

In some embodiments, the Th1 cytokines comprise interferon-gamma (IFN-γ), IL-2R_{α}, IL-12p70, and IL-2; the chemokine(s) or adhesion molecules comprise: a C—C motif chemokine ligand 2 (CCL2) monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8; the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α; the regulatory mediator molecules comprise: native transforming growth factor beta (native TGF-β), an interleukin-1 receptor antagonist (IL-1RA), a total transforming growth factor beta (total TGF-β), and IL-10; the SLE mediator molecules comprise: a stem cell factor (SCF) and Resistin; and wherein the biomarkers further comprise: innate cytokines, wherein the innate cytokines comprise: IL-7, IL-1α, and IL-1β; T-helper type-2 (Th2) cytokines, wherein the Th2 cytokines comprise: IL-13 and IL-4; and one or more Th17 cytokines, wherein the one or more Th17 cytokines comprise IL-17A. In some embodiments, the expression levels of biomarkers comprise protein levels. In some embodiments, the expression levels of biomarkers are determined using one of an ELISA assay, xMAP® technology, or SimplePlex™ assay. In some embodiments, the expression levels of biomarkers comprise mRNA levels. In some embodiments, the mRNA levels are obtained from circulating cells. In some embodiments, the mRNA levels are obtained from circulating T-cells.

In some embodiments, the instructions that cause the processor to perform the step of generating the LFPI based on the expression levels comprise instructions that, when executed by the processor, cause the processor to perform the step of applying a predictive model. In some embodiments, the instructions that cause the processor to perform the step of applying the predictive model comprise instructions that, when executed by the processor, cause the processor to perform the steps of: for the expression level of each biomarker: log-transforming the expression level; standardizing the expression level; obtaining a corresponding coefficient for the biomarker; the corresponding coefficient representing an association between pre-flare expression levels of the biomarker and a measurement of SLE clinical disease activity: and weighting the standardized expression level with the corresponding coefficient to obtain a LFPI subscore for the biomarker; and summing the LFPI subscores to obtain the LFPI. In some embodiments, for each expression level, the corresponding coefficient is obtained from a linear regression model testing associations between the measurement of SLE clinical disease activity and the pre-flare expression levels of the biomarker. In some embodiments, the instructions that cause the processor to perform the step of standardizing the expression level further comprises instructions that, when executed by the processor, cause the processor to perform the step of normalizing the expression level to a mean expression value for SLE patients with stable SLE disease.

In some embodiments, the biomarkers are selected for inclusion in the dataset using an applied machine learning modeling approach. In some embodiments, the applied machine learning modeling approach is one of random forest or gradient boosting. In some embodiments, the measurement of SLE clinical disease activity is the Safety of Estrogens in Lupus Erythematosus National Assessment-Systemic Lupus Erythematosus Disease Activity Index (SELENA-SLEDAI). In some embodiments, the measurement of SLE clinical disease activity is determined from samples obtained from a group of patients undergoing a flare event. In some embodiments, performance of the predictive model is characterized by an area under a receiver operating characteristic curve that is greater than 0.85. In some embodiments, performance of the predictive model is characterized by an area under a receiver operating characteristic curve that is greater than 0.90. In some embodiments, the future SLE disease activity event is one of a future flare event or future organ damage. In some embodiments, the dataset further comprises expression levels of biomarkers from a second test sample taken from the systemic lupus erythematosus (SLE) subject at a different time point.

Additionally disclosed herein is a method comprising: (a) obtaining a blood, serum, or plasma sample from the SLE subject; (b) assessing expression levels of biomarkers from a test sample from the SLE subject, wherein the biomarkers comprise: at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin. In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ). In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-β), and Intercellular Adhesion Molecule 1 (ICAM-1); the at least two TNFR superfamily member molecules comprise tumor necrosis factor receptor I (TNFRI), tumor necrosis factor receptor II (TNFRI), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

Additionally disclosed herein is a method comprising: (a) obtaining a blood, serum, or plasma sample from the SLE subject; (b) assessing expression levels of biomarkers from a test sample from the SLE subject, wherein the biomarkers comprise: at least one innate cytokine selected from IL-7, IL-1α, and IL-1β; at least one Th1 cytokine selected from interferon-gamma (IFN-γ), IL-12p70, IL-2, and IL-2R_{α}, at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β). Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; (b) generating a LFPI based on the expression levels in the obtained dataset; and (c) determining likelihood of a future SLE disease activity event in the SLE subject based on the LFPI.

Additionally disclosed herein is a method for assessing expression levels in a systemic lupus erythematosus (SLE) subject comprising: (a) obtaining a blood, serum, or plasma sample from the SLE subject; (b) assessing expression levels of biomarkers from a test sample from the SLE subject, wherein the biomarkers comprise: chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); tumor necrosis factor receptor (TNFR) superfamily member molecules, wherein the TNFR superfamily member molecules comprise: a tumor necrosis factor receptor I (TNFRI), and a tumor necrosis factor receptor II (TNFRII): regulatory mediator molecules, wherein the regulatory mediator molecules comprise: native transforming growth factor beta (native TGF-β), and an interleukin-1 receptor antagonist (IL-1RA); and one or more systemic lupus erythematosus (SLE) mediator molecules, wherein the one or more SLE mediator molecules comprise a stem cell factor (SCF). In some embodiments, the biomarkers further comprise one or more T-helper type-1 (Th1) cytokines, wherein the one or more Th1 cytokines comprise an interferon-gamma (IFN-γ). In some embodiments, the tumor necrosis factor receptor (TNFR) superfamily member molecules further comprise a tumor necrosis factor-related apoptosis-inducing ligand (TRAIL). In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1). In some embodiments, the regulatory mediator molecules further comprise total TGF-β. In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1); the TNFR superfamily member molecules further comprise a tumor necrosis factor-related apoptosis-inducing ligand (TRAIL); and the regulatory mediator molecules further comprise a total transforming growth factor beta (total TGF-β). In some embodiments, the biomarkers further comprise one or more innate cytokines, wherein the one or more innate cytokines are selected from IL-7, IL-1α, and IL-1β. In some embodiments, the biomarkers further comprise one or more T-helper type-2 (Th2) cytokines, wherein the one or more Th2 cytokines are selected from IL-13 and IL-4. In some embodiments, the biomarkers further comprise one or more Th17 cytokines, wherein the one or more Th17 cytokines comprise IL-17A. In some embodiments, the one or more SLE mediator molecules further comprise Resistin. In some embodiments, the Th1 cytokines further comprise IL-2, IL-12p70, and IL-2R_{α}. In some embodiments, the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10. In some embodiments, the Th1 cytokines comprise interferon-gamma (IFN-γ), IL-2R_{α} IL-12p70, and IL-2; the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8; the TNFR superfamily member molecules comprise: a tumor necrosis factor receptor I (TNFRI), a tumor necrosis factor receptor II (TNFRII), a tumor necrosis factor-related apoptosis-inducing ligand (TRAIL). Fas, NGF-β, and TNF-α; the regulatory mediator molecules comprise: native transforming growth factor beta (native TGF-β), an interleukin-1 receptor antagonist (IL-1RA), a total transforming growth factor beta (total TGF-β), and IL-10; the SLE mediator molecules comprise: a stem cell factor (SCF) and Resistin; and wherein the biomarkers further comprise: innate cytokines, wherein the innate cytokines comprise: IL-7, IL-1α, and IL-1β; T-helper type-2 (Th2) cytokines, wherein the Th2 cytokines comprise: IL-13 and IL-4; and one or more Th17 cytokines, wherein the one or more Th17 cytokines comprise IL-17A.

In some embodiments, the expression levels of biomarkers comprise protein levels. In some embodiments, the expression levels of biomarkers are determined using one of an ELISA assay, xMAPS® technology, or SimplePlex™ assay. In some embodiments, the expression levels of biomarkers comprise mRNA levels. In some embodiments, the mRNA levels are obtained from circulating cells. In some embodiments, the mRNA levels are obtained from circulating T-cells.

In some embodiments, the method further comprises: determining a likelihood that the SLE subject will have a future SLE disease activity event, wherein the determination comprises: determining that expression levels of the Th1, chemokine/adhesion molecules, and TNFR superfamily member molecules are elevated and that expression levels of the regulator mediator molecules are reduced as compared to expression levels in a previous sample from the SLE subject. In some embodiments, the method further comprises administering a treatment to the SLE subject after determining that the SLE subject is likely to have the future SLE disease activity event. In some embodiments, the method further comprises: generating a LFPI based on the assessed expression levels. In some embodiments, generating the LFPI based on the expression levels comprises applying a predictive model. In some embodiments, applying the predictive model comprises, for the assessed expression level of each biomarker: log-transforming the expression level; standardizing the expression level; obtaining a corresponding coefficient for the biomarker; the corresponding coefficient representing an association between pre-flare expression levels of the biomarker and a measurement of SLE clinical disease activity; and weighting the standardized expression level with the corresponding coefficient to obtain a LFPI subscore for the biomarker; and summing the LFPI subscores to obtain the LFPI. In some embodiments, for each assessed expression level, the corresponding coefficient is obtained from a linear regression model testing associations between the measurement of SLE clinical disease activity and the pre-flare expression levels of the biomarker. In some embodiments, standardizing the assessed expression level comprises normalizing the expression level to a mean expression value for SLE patients with stable SLE disease. In some embodiments, the measurement of SLE clinical disease activity is the Safety of Estrogens in Lupus Erythematosus National Assessment-Systemic Lupus Erythematosus Disease Activity Index (SELENA-SLEDAI).

In some embodiments, the measurement of SLE clinical disease activity is determined from samples obtained from a group of patients undergoing a flare event. In some embodiments, performance of the predictive model is characterized by an area under a receiver operating characteristic curve that is greater than 0.85. In some embodiments, performance of the predictive model is characterized by an area under a receiver operating characteristic curve that is greater than 0.90. In some embodiments, the future SLE disease activity event is one of a future flare event or future organ damage.

Additionally disclosed herein is a computer system for assessing likelihood of a future SLE disease activity event in a systemic lupus erythematosus (SLE) subject, the computer system comprising: a storage memory for storing a dataset comprising expression levels for biomarkers from a test sample from the SLE subject, the biomarkers comprising: at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; a processor communicatively coupled to the storage memory for determining a LFPI by applying a predictive model to the stored dataset, the LFPI predictive of the likelihood of the future SLE disease activity event in the SLE subject. In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ). In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG). C—C motif chemokine ligand 4 (CCL4)/macrophage inflammatory protein-1 beta (MIP-1P), and Intercellular Adhesion Molecule 1 (ICAM-1): the at least two TNFR superfamily member molecules comprise tumor necrosis factor receptor I (TNFRI), tumor necrosis factor receptor II (TNFRII), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

Additionally disclosed herein is a computer system for assessing likelihood of a future SLE disease activity event in a systemic lupus erythematosus (SLE) subject, the computer system comprising: a storage memory for storing a dataset comprising expression levels for biomarkers from a test sample from the SLE subject, the biomarkers comprising: at least one innate cytokine selected from IL-7, IL-1α, and IL-1β; at least one Th1 cytokine selected from interferon-gamma (IFN-γ), IL-12p70, IL-2, and IL-2R_{α}; at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; a processor communicatively coupled to the storage memory for determining a LFPI by applying a predictive model to the stored dataset, the LFPI predictive of the likelihood of the future SLE disease activity event in the SLE subject.

Additionally disclosed herein is a computer system for assessing likelihood of a future SLE disease activity event in a systemic lupus erythematosus (SLE) subject, the computer system comprising: a storage memory for storing a dataset comprising expression levels for biomarkers from a test sample from the SLE subject, the biomarkers comprising: (i) chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); (ii) tumor necrosis factor receptor (TNFR) superfamily member molecules, wherein the TNFR superfamily member molecules comprise: a tumor necrosis factor receptor I (TNFRI), and a tumor necrosis factor receptor II (TNFRII); (iii) regulatory mediator molecules, wherein the regulatory mediator molecules comprise: native transforming growth factor beta (native TGF-β), and an interleukin-1 receptor antagonist (IL-1RA); and (iv) one or more systemic lupus erythematosus (SLE) mediator molecules, wherein the one or more SLE mediator molecules comprise a stem cell factor (SCF); and a processor communicatively coupled to the storage memory for determining a LFPI by applying a predictive model to the stored dataset, the LFPI predictive of the likelihood of the future SLE disease activity event in the SLE subject. In some embodiments, the biomarkers further comprise one or more T-helper type-1 (Th1) cytokines, wherein the one or more Th1 cytokines comprise an interferon-gamma (IFN-γ). In some embodiments, the tumor necrosis factor receptor (TNFR) superfamily member molecules further comprise a tumor necrosis factor-related apoptosis-inducing ligand (TRAIL). In some embodiments, the chemokine(s) or adhesion molecules further comprise: a C—C motif chemokine ligand 4 (CCL4)/macrophage inflammatory protein-1 beta (MIP-1β) and an Intercellular Adhesion Molecule 1 (ICAM-1). In some embodiments, the regulatory mediator molecules further comprise total TGF-β. In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1); the TNFR superfamily member molecules further comprise a tumor necrosis factor-related apoptosis-inducing ligand (TRAIL); and the regulatory mediator molecules further comprise a total transforming growth factor beta (total TGF-β). In some embodiments, the biomarkers further comprise (vi) one or more innate cytokines, wherein the one or more innate cytokines are selected from IL-7, IL-1α, and IL-1β. In some embodiments, the biomarkers further comprise (vii) one or more T-helper type-2 (Th2) cytokines, wherein the one or more Th2 cytokines are selected from IL-13 and IL-4. In some embodiments, the biomarkers further comprise (viii) one or more Th17 cytokines, wherein the one or more Th17 cytokines comprise IL-17A. In some embodiments, the one or more SLE mediator molecules further comprise Resistin. In some embodiments, the Th1 cytokines further comprise IL-2, IL-12p70, and IL-2R_{α}. In some embodiments, the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10. In some embodiments, the Th1 cytokines comprise interferon-gamma (IFN-γ), IL-2R_{α} IL-12p70, and IL-2; the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8; the TNFR superfamily member molecules comprise: a tumor necrosis factor receptor I (TNFRI), a tumor necrosis factor receptor II (TNFRII), a tumor necrosis factor-related apoptosis-inducing ligand (TRAIL). Fas, NGF-β, and TNF-α; the regulatory mediator molecules comprise: native transforming growth factor beta (native TGF-β), an interleukin-1 receptor antagonist (IL-1RA), a total transforming growth factor beta (total TGF-β), and IL-10; the SLE mediator molecules comprise: a stem cell factor (SCF) and Resistin; and wherein the biomarkers further comprise: innate cytokines, wherein the innate cytokines comprise: IL-7, IL-1α, and IL-1β; T-helper type-2 (Th2) cytokines, wherein the Th2 cytokines comprise: IL-13 and IL-4; and one or more Th17 cytokines, wherein the one or more Th17 cytokines comprise IL-17A.

In some embodiments, the expression levels of biomarkers comprise protein levels. In some embodiments, the expression levels of biomarkers are determined using one of an ELISA assay, xMAP® technology, or SimplePlex™ assay. In some embodiments, the expression levels of biomarkers comprise mRNA levels. In some embodiments, the mRNA levels are obtained from circulating cells. In some embodiments, the mRNA levels are obtained from circulating T-cells. In some embodiments, applying the predictive model to the stored dataset comprises, for the expression level of each biomarker: log-transforming the expression level; standardizing the expression level; obtaining a corresponding coefficient for the biomarker; the corresponding coefficient representing an association between pre-flare expression levels of the biomarker and a measurement of SLE clinical disease activity; and weighting the standardized expression level with the corresponding coefficient to obtain a LFPI subscore for the biomarker; and summing the LFPI subscores to obtain the LFPI. In some embodiments, for each expression level, the corresponding coefficient is obtained from a linear regression model testing associations between the measurement of SLE clinical disease activity and the pre-flare expression levels of the biomarker. In some embodiments standardizing the expression level comprises normalizing the expression level to a mean expression value for SLE patients with stable SLE disease. In some embodiments, the measurement of SLE clinical disease activity is the Safety of Estrogens in Lupus Erythematosus National Assessment-Systemic Lupus Erythematosus Disease Activity Index (SELENA-SLEDAI).

In some embodiments, the measurement of SLE clinical disease activity is determined from samples obtained from a group of patients undergoing a flare event. In some embodiments, performance of the predictive model is characterized by an area under a receiver operating characteristic curve that is greater than 0.85. In some embodiments, performance of the predictive model is characterized by an area under a receiver operating characteristic curve that is greater than 0.90. In some embodiments, the future SLE disease activity event is one of a future flare event or future organ damage. In some embodiments, the dataset further comprises expression levels of biomarkers from a second test sample taken from the systemic lupus erythematosus (SLE) subject at a different time point.

Additionally disclosed herein is a kit for assessing likelihood of a future SLE disease activity event in a systemic lupus erythematosus (SLE) subject, the kit comprising: a set of reagents for determining expression levels for biomarkers from a test sample from the SLE subject, wherein the biomarkers comprise: at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; and instructions for using the set of reagents to determine the expression levels of biomarkers from the test sample. In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ). In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1); the at least two TNFR superfamily member molecules comprise tumor necrosis factor receptor I (TNFRI), tumor necrosis factor receptor II (TNFRI), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

Additionally disclosed herein is a kit for assessing likelihood of a future SLE disease activity event in a systemic lupus erythematosus (SLE) subject, the kit comprising: a set of reagents for determining expression levels for biomarkers from a test sample from the SLE subject, wherein the biomarkers comprise: at least one innate cytokine selected from IL-7, IL-1α, and IL-1β; at least one Th1 cytokine selected from interferon-gamma (IFN-γ), IL-12p70, IL-2, and IL-2R_{α}; at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; and instructions for using the set of reagents to determine the expression levels of biomarkers from the test sample.

Additionally disclosed herein is a kit for assessing likelihood of a future SLE disease activity event in a systemic lupus erythematosus (SLE) subject, the kit comprising: a set of reagents for determining expression levels for biomarkers from a test sample from the SLE subject, wherein the biomarkers comprise: (i) chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); (ii) tumor necrosis factor receptor (TNFR) superfamily member molecules, wherein the TNFR superfamily member molecules comprise: a tumor necrosis factor receptor I (TNFRI), and a tumor necrosis factor receptor II (TNFRII); (iii) regulatory mediator molecules, wherein the regulatory mediator molecules comprise: native transforming growth factor beta (native TGF-β), and an interleukin-1 receptor antagonist (IL-1RA); and (iv) one or more systemic lupus erythematosus (SLE) mediator molecules, wherein the one or more SLE mediator molecules comprise a stem cell factor (SCF); and instructions for using the set of reagents to determine the expression levels of biomarkers from the test sample. In some embodiments, the biomarkers further comprise one or more T-helper type-1 (Th1) cytokines, wherein the one or more Th1 cytokines comprise an interferon-gamma (IFN-γ). In some embodiments, the tumor necrosis factor receptor (TNFR) superfamily member molecules further comprise a tumor necrosis factor-related apoptosis-inducing ligand (TRAIL). In some embodiments, the chemokine(s) or adhesion molecules further comprise: a C—C motif chemokine ligand 4 (CCL4)/macrophage inflammatory protein-1 beta (MIP-1β) and an Intercellular Adhesion Molecule 1 (ICAM-1). In some embodiments, the regulatory mediator molecules further comprise total TGF-β. In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1); the TNFR superfamily member molecules further comprise a tumor necrosis factor-related apoptosis-inducing ligand (TRAIL); and the regulatory mediator molecules further comprise a total transforming growth factor beta (total TGF-β). In some embodiments, the biomarkers further comprise (vi) one or more innate cytokines, wherein the one or more innate cytokines are selected from IL-7, IL-1α, and IL-1β. In some embodiments, the biomarkers further comprise (vii) one or more T-helper type-2 (Th2) cytokines, wherein the one or more Th2 cytokines are selected from IL-13 and IL-4. In some embodiments, the biomarkers further comprise (viii) one or more Th17 cytokines, wherein the one or more Th17 cytokines comprise IL-17A. In some embodiments, the one or more SLE mediator molecules further comprise Resistin. In some embodiments, the Th1 cytokines further comprise IL-2, IL-12p70, and IL-2R_{α}. In some embodiments, the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10. In some embodiments, the Th1 cytokines comprise interferon-gamma (IFN-γ), IL-2R_{α} IL-12p70, and IL-2; the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8; the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α; the regulatory mediator molecules comprise: native transforming growth factor beta (native TGF-β), an interleukin-1 receptor antagonist (IL-1RA), a total transforming growth factor beta (total TGF-β), and IL-10; the SLE mediator molecules comprise: a stem cell factor (SCF) and Resistin: and wherein the biomarkers further comprise: innate cytokines, wherein the innate cytokines comprise: IL-7, IL-1α, and IL-1β; T-helper type-2 (Th2) cytokines, wherein the Th2 cytokines comprise: IL-13 and IL-4; and one or more Th17 cytokines, wherein the one or more Th17 cytokines comprise IL-17A.

In some embodiments, the expression levels of biomarkers comprise protein levels. In some embodiments, the expression levels of biomarkers are determined using one of an ELISA assay, xMAP® technology, or SimplePlex™ assay. In some embodiments, the expression levels of biomarkers comprise mRNA levels. In some embodiments, the mRNA levels are obtained from circulating cells. In some embodiments, the mRNA levels are obtained from circulating T-cells. In some embodiments, the instructions further comprise instructions for determining a LFPI from the expression levels by applying a predictive model, the LFPI predictive of the likelihood of the future SLE disease activity event in the SLE subject. In some embodiments, applying the predictive model comprises, for the expression level of each biomarker: log-transforming the expression level; standardizing the expression level; obtaining a corresponding coefficient for the biomarker; the corresponding coefficient representing an association between pre-flare expression levels of the biomarker and a measurement of SLE clinical disease activity; and weighting the standardized expression level with the corresponding coefficient to obtain a LFPI subscore for the biomarker; and summing the LFPI subscores to obtain the LFPI. In some embodiments, for each expression level, the corresponding coefficient is obtained from a linear regression model testing associations between the measurement of SLE clinical disease activity and the pre-flare expression levels of the biomarker. In some embodiments, standardizing the expression level comprises normalizing the expression level to a mean expression value for SLE patients with stable SLE disease. In some embodiments, the measurement of SLE clinical disease activity is the Safety of Estrogens in Lupus Erythematosus National Assessment-Systemic Lupus Erythematosus Disease Activity Index (SELENA-SLEDAI).

In some embodiments, the measurement of SLE clinical disease activity is determined from samples obtained from a group of patients undergoing a flare event. In some embodiments, performance of the predictive model is characterized by an area under a receiver operating characteristic curve that is greater than 0.85. In some embodiments, performance of the predictive model is characterized by an area under a receiver operating characteristic curve that is greater than 0.90. In some embodiments, the future SLE disease activity event is one of a future flare event or future organ damage.

Additionally disclosed herein is a system for assessing likelihood of a future SLE disease activity event in a systemic lupus erythematosus (SLE) subject, the system comprising: a set of reagents used for determining expression levels for biomarkers from a test sample from the SLE subject, wherein the biomarkers comprise: at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; an apparatus configured to receive a mixture of one or more reagents in the set and the test sample and to measure the expression levels for the biomarkers from the test sample; and a computer system communicatively coupled to the apparatus to obtain a dataset comprising the measured expression levels for the biomarkers from the test sample and to determine a LFPI by applying a predictive model to the dataset, the LFPI predictive of the likelihood of the future SLE disease activity event in the SLE subject. In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ). In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1), the at least two TNFR superfamily member molecules comprise tumor necrosis factor receptor I (TNFRI), tumor necrosis factor receptor II (TNFRII), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

Additionally disclosed herein is a system for assessing likelihood of a future SLE disease activity event in a systemic lupus erythematosus (SLE) subject, the system comprising: a set of reagents used for determining expression levels for biomarkers from a test sample from the SLE subject, wherein the biomarkers comprise: at least one innate cytokine selected from IL-7, IL-1α, and IL-1p; at least one Th1 cytokine selected from interferon-gamma (IFN-γ), IL-12p70, IL-2, and IL-2R_{α}; at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β). Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRI), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin; an apparatus configured to receive a mixture of one or more reagents in the set and the test sample and to measure the expression levels for the biomarkers from the test sample; and a computer system communicatively coupled to the apparatus to obtain a dataset comprising the measured expression levels for the biomarkers from the test sample and to determine a LFPI by applying a predictive model to the dataset, the LFPI predictive of the likelihood of the future SLE disease activity event in the SLE subject.

Additionally disclosed herein is a system for assessing likelihood of a future SLE disease activity event in a systemic lupus erythematosus (SLE) subject, the system comprising: a set of reagents used for determining expression levels for biomarkers from a test sample from the SLE subject, wherein the biomarkers comprise: (i) chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); (ii) tumor necrosis factor receptor (TNFR) superfamily member molecules, wherein the TNFR superfamily member molecules comprise: a tumor necrosis factor receptor I (TNFRI), and a tumor necrosis factor receptor II (TNFRII); (iii) regulatory mediator molecules, wherein the regulatory mediator molecules comprise: native transforming growth factor beta (native TGF-β), and an interleukin-1 receptor antagonist (IL-1RA); and (iv) one or more systemic lupus erythematosus (SLE) mediator molecules, wherein the one or more SLE mediator molecules comprise a stem cell factor (SCF); an apparatus configured to receive a mixture of one or more reagents in the set and the test sample and to measure the expression levels for the biomarkers from the test sample; and a computer system communicatively coupled to the apparatus to obtain a dataset comprising the measured expression levels for the biomarkers from the test sample and to determine a LFPI by applying a predictive model to the dataset, the LFPI predictive of the likelihood of the future SLE disease activity event in the SLE subject.

In some embodiments, the biomarkers further comprise (i) one or more T-helper type-1 (Th1) cytokines, wherein the one or more Th1 cytokines comprise an interferon-gamma (IFN-γ). In some embodiments, the tumor necrosis factor receptor (TNFR) superfamily member molecules further comprise a tumor necrosis factor-related apoptosis-inducing ligand (TRAIL). In some embodiments, the chemokine(s) or adhesion molecules further comprise: a C—C motif chemokine ligand 4 (CCL4)/macrophage inflammatory protein-1 beta (MIP-1β) and an Intercellular Adhesion Molecule 1 (ICAM-1). In some embodiments, the regulatory mediator molecules further comprise total TGF-β. In some embodiments, the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1); the TNFR superfamily member molecules further comprise a tumor necrosis factor-related apoptosis-inducing ligand (TRAIL); and the regulatory mediator molecules further comprise a total transforming growth factor beta (total TGF-β). In some embodiments, the biomarkers further comprise (vi) one or more innate cytokines, wherein the one or more innate cytokines are selected from IL-7, IL-1α, and IL-1β. In some embodiments, the biomarkers further comprise (vii) one or more T-helper type-2 (Th2) cytokines, wherein the one or more Th2 cytokines are selected from IL-13 and IL-4. In some embodiments, the biomarkers further comprise (viii) one or more Th17 cytokines, wherein the one or more Th17 cytokines comprise IL-17A. In some embodiments, the one or more SLE mediator molecules further comprise Resistin. In some embodiments, the Th1 cytokines further comprise IL-2, IL-12p70, and IL-2R_{α}. In some embodiments, the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

In some embodiments, the Th1 cytokines comprise interferon-gamma (IFN-γ), IL-2R_{α}, IL-12p70, and IL-2; the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8; the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α; the regulatory mediator molecules comprise: native transforming growth factor beta (native TGF-β), an interleukin-1 receptor antagonist (IL-1RA), a total transforming growth factor beta (total TGF-β), and IL-10; the SLE mediator molecules comprise: a stem cell factor (SCF) and Resistin; and wherein the biomarkers further comprise: innate cytokines, wherein the innate cytokines comprise: IL-7, IL-1α, and IL-1β; T-helper type-2 (Th2) cytokines, wherein the Th2 cytokines comprise: IL-13 and IL-4; and one or more Th17 cytokines, wherein the one or more Th17 cytokines comprise IL-17A.

In some embodiments, the expression levels comprise protein levels. In some embodiments, the expression levels of biomarkers are determined using one of an ELISA assay, xMAP® technology, or SimplePlex™ assay. In some embodiments, the expression levels comprise mRNA levels. In some embodiments, the mRNA levels are obtained from circulating cells. In some embodiments, the mRNA levels are obtained from circulating T-cells. In some embodiments, applying the predictive model to the dataset comprises, for the expression level of each biomarker: log-transforming the expression level; standardizing the expression level; obtaining a corresponding coefficient for the biomarker; the corresponding coefficient representing an association between pre-flare expression levels of the biomarker and a measurement of SLE clinical disease activity; and weighting the standardized expression level with the corresponding coefficient to obtain a LFPI subscore for the biomarker; and summing the LFPI subscores to obtain the LFPI. In some embodiments, for each expression level, the corresponding coefficient is obtained from a linear regression model testing associations between the measurement of SLE clinical disease activity and the pre-flare expression levels of the biomarker. In some embodiments, standardizing the expression level comprises normalizing the expression level to a mean expression value for SLE patients with stable SLE disease. In some embodiments, the measurement of SLE clinical disease activity is the Safety of Estrogens in Lupus Erythematosus National Assessment-Systemic Lupus Erythematosus Disease Activity Index (SELENA-SLEDAI). In some embodiments, the measurement of SLE clinical disease activity is determined from samples obtained from a group of patients undergoing a flare event.

In some embodiments, performance of the predictive model is characterized by an area under a receiver operating characteristic curve that is greater than 0.85. In some embodiments, performance of the predictive model is characterized by an area under a receiver operating characteristic curve that is greater than 0.90. In some embodiments, performance of the predictive model is characterized by an area under a receiver operating characteristic curve that is greater than 0.94. In some embodiments, the future SLE disease activity event is one of a future flare event or future organ damage.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 depicts a Lupus Flare Prediction Index (LFPI) informed by 31 soluble mediators in SLE patients with impending clinical disease flare. A. LFPI scores from baseline (Pre-flare/Pre-self non-flare (Pre-SNF)) plasma levels were determined for SLE patients who exhibited clinical disease flare at their follow-up clinic visit compared to the same SLE patients in comparable series of clinic visits with no observed disease flare (self non-flare (SNF), p<0.0001 by Wilcoxon matched-pairs test). Data presented as Box and Whisker (median±max and min) graphs. B. Receiving operating characteristic (ROC) curve to determine area under the curve (AUC) for the LFPI. C. LFPI scores for each SLE patient were compared between year of impending clinical disease flare (Flare) and period of non-flare (SNF).

FIG. 2 depicts variable importance within mediators that differentiate Pre-flare and Pre-SNF samples. Random forest (A) and XG Boost (B) were run 2000 times on ⅔ (train) and ⅓ (test), randomly generated, subsets of data as described in Methods. Analytes were ranked in order of decreased Gini f SD (random forest, A) or Gain±SD (XGBoost, B). True negative (TN), false positive (FP), false negative (FN), and true positive (TP) samples, as well as accuracy, sensitivity, specificity, precision, and negative predictive value (NPV) are shown for the test and train sets for each algorithm.

FIG. 3 depicts the number and types of mediators informing the LFPI that determines LFPI performance. Forward and backward step-wise progression, based on random forest Variable Importance, was performed to determine window of number/type of soluble mediator for optimal LFPI performance, including LFPI Pre-flare vs. Pre-SNF delta values (A), receiver operating characteristic (ROC) curves (B), LFPI performance with respect to sensitivity, specificity, positive predictive value (PPV), negative predictive value (NPV), and accuracy (C), and odds ratio (OR)±95% confidence interval (CI) that reflects the likelihood of a Pre-flare sample having a positive LFPI score (D).

FIG. 4 depicts Lupus Flare Prediction Index (LFPI) informed by 10 mediators in SLE patients with impending clinical disease flare. A. LFPI scores from baseline (Pre-flare/Pre-SNF) plasma levels were determined for SLE patients who exhibited disease flare at their follow-up clinic visit compared to the same SLE patients in comparable series of clinic visits with no observed disease flare (SNF, p<0.0001 by Wilcoxon matched-pairs test). Data presented as Box and Whisker (median±max and min) graphs. B. Receiving operating characteristic (ROC) curve to determine area under the curve (AUC) for the LFPI. C. LFPI scores for each SLE patient were compared between year of impending disease flare (Flare) and period of non-flare (SNF).

FIG. 5 depicts altered plasma soluble mediator levels in Pre-flare vs. Pre-SNF samples. Top mediators, as determined by random forest Variable Importance, were evaluated for their levels between Pre-flare vs. Pre-SNF samples in the same SLE patients, including IFN-associated mediators, IFN-γ, IP-10, MCP-1, MIG, MIP-1α, and MIP-1β (A), inflammatory (SCF) and TNF receptors, TNFRI and TNFRII (B), and regulatory mediators, IL-1RA, Active TGF-β, and Total TGF-β (C). Bars are reflective of mean±SEM. p<0.0001 by Wilcoxon matched pairs test for all analytes.

FIG. 6 depicts altered baseline LFPI and soluble mediator levels in SLE patients with select organ system manifestations at follow-up. The 10-mediator informed LFPI (A), as well as SCF (B), IP-10/CXCL10 (C), IL-1RA (D), and Active TGF-β (E) in plasma samples at baseline in SLE patients with either arthritis (n=59 with arthritis, n=116 without arthritis), mucocutaneous (MC; rash, alopecia, or mucosal ulcers; n=93 with MC, n=86 without MC), or serositis (n=8 with serositis, n=172 without serositis) at follow-up clinic visit. Bars are reflective of mean±SEM. *p≤0.05, **p≤<0.01, ***p≤<0.001, ****p<0.0001 by Mann-Whitney test.

FIGS. 7A and 7B depict embodiments of using predictive models for predicting a score based on soluble mediator expression levels.

FIG. 8 depicts an example timeline of monitoring an individual in a cohort of SLE patients.

FIG. 9 depicts an example computer for implementing a predictive model disclosed herein, e.g., a predictive model shown in FIG. 7A or 7B.

### DETAILED DESCRIPTION OF THE INVENTION

### Definitions

Terms used in the claims and specification are defined as set forth below unless otherwise specified.

The term “subject” encompasses a cell, tissue, or organism, human or non-human, whether in vivo, ex vivo, or in vitro, male or female.

The terms “marker,” “markers,” “biomarker,” “biomarkers,” “soluble mediator,” and “soluble mediators” are used interchangeably and encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.). Example biomarkers referred to in various embodiments include, but are not limited to, protein biomarkers documented in Table 4 and identified using UnitProt Entry Identifier accessed on Sep. 4, 2019.

The term “SLE disease activity event” as used herein refers to a future SLE flare event or future organ system damage or organ system inflammation due to SLE in a SLE subject. Examples of organ system inflammation/damage include central nervous system inflammation/damage (e.g. seizure, psychosis, organic brain syndrome, visual disturbance, cranial nerve disorder, or lupus headache, or cerebrovascular accident [CVA] due to SLE), arthritis, myositis, renal sequelae (e.g. urinary casts, hematuria, proteinuria, or pyuria due to SLE), mucotaneous disorders (e.g. rash, alopecia, or mucosal ulcers due to SLE), serositis (e.g. pleurisy or pericarditis due to SLE), low complement, increased DNA binding, fever, thrombocytopenia, or leukopenia directly due to SLE pathogenesis. The phrase “likelihood of SLE disease activity event” refers to the likelihood of an impending SLE flare or likelihood of future organ system damage due to SLE in a SLE subject.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “obtaining a dataset associated with a sample” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

The term “pre-flare expression levels” refers to expression levels of soluble mediators determined from a pre-flare sample taken from a SLE patient, who is not experiencing a clinical disease flare, a number of days prior to the patient experiencing a clinical flare event. In some embodiments, the number of days prior to the patient experiencing a clinical flare event is 30 days, 60 days, 90 days, 120 days, 150 days, or 180 days. In some embodiments, the number of days prior to the patient experiencing a clinical flare event is between 30 and 150 days, between 50 and 120 days, or between 75 and 100 days.

The term “pre-SNF expression levels” refers to expression levels of soluble mediators determined from a pre-SNF (self non-flare) sample taken from a SLE patient, who is not experiencing a clinical disease flare, a number of days prior to the patient also not experiencing a flare event. In some embodiments, the number of days prior to the patient also not experiencing a clinical flare event is 30 days, 60 days, 90 days, 120 days, 150 days, or 180 days. In some embodiments, the number of days prior to the patient also not experiencing a clinical flare event is between 30 and 150 days, between 50 and 120 days, or between 75 and 100 days.

It must be noted that, as used in the specification and the appended claims, the singular forms “a,” “an” and “the” include plural referents unless the context clearly dictates otherwise.

Overview

Provided herein are methods, kits, non-transitory computer readable mediums, computer systems, and systems for predicting a likelihood of a SLE disease activity event in a subject. The methods, kits, and systems disclosed herein generally employ a panel of biomarkers to predict likelihood of a future SLE disease activity event. In some embodiments, the panel of biomarkers comprise chemokine(s) or adhesion molecules, TNFR superfamily member molecules, regulatory mediator molecules, and SLE mediator molecules.

In some embodiments, the prediction comprises generating a LFPI subscore representing a likelihood of the SLE disease activity event based on expression level of one soluble mediator. In some embodiments, the prediction comprises generating a LFPI. The LFPI refers to a likelihood of a future SLE disease activity event based on the expression levels of multiple soluble mediators. As one example, the LFPI can represent a combination of multiple LFPI subscores.

The present methods, kits, non-transitory computer readable mediums, computer systems, and systems can predict an SLE disease occurrence in advance. In some embodiments, the present methods, kits, non-transitory computer readable mediums, computer systems, and systems implement biomarker panel testing which enables prediction of a SLE disease activity event 30 days or more in advance, e.g., 30 days, 60 days, 90 days, 120 days, 150 days, or 180 days prior to occurrence of the SLE disease activity event. In some embodiments, the biomarker panel testing enables prediction of a SLE disease activity event between 30 days and 90 days, between 50 and 120 days, or between 75 and 100 days prior to occurrence of the SLE disease activity event.

Advantages and Utility

The present methods, kits, and systems confer numerous advantages. The Lupus Flare Prediction Index (LFPI), as described in further detail below, enables monitoring of the overall immune status and differentiate subjects that are likely to experience a SLE disease activity event (e.g., a future flare or future organ damage). By providing a broad survey of immune pathway activation, the LFPI demonstrates consistency despite immunologic and clinical heterogeneity among patients. In other words, the combination of biomarkers used for determining the LFPI represents an unexpected combination of soluble mediators for the purposes of accurately predicting a SLE disease activity event ahead of its occurrence, therefore enabling improved patient treatment and outcomes.

The ability to identify patients at risk of a SLE disease activity event could optimize the timing of disease suppression therapy and contribute to more effective and efficient clinical trial designs. This could improve patient outcomes and reduce the pathogenic and socioeconomic burdens of SLE (30). An advantage of calculating a patient's LFPI is that it does not require cut-offs for each soluble mediator to establish positivity and does not require a priori knowledge of the inflammatory pathways that contribute to flare in a particular patient.

Methods

The methods disclosed herein may include obtaining and analyzing expression levels of one or more soluble mediators in a sample obtained from a subject to predict a likelihood of a SLE disease activity event (e.g., likelihood of impending flare or likelihood of organ inflammation). In various embodiments, methods include obtaining or having obtained a dataset that includes the expression levels of one or more soluble mediators. In one aspect, obtaining a dataset includes obtaining a sample from a subject (e.g., a SLE patient) and processing the sample to experimentally to assess the expression levels of the one or more soluble mediators. In some embodiments, processing the sample includes performing an immunologic assay. In some embodiments, processing the sample includes performing an assay for detecting nucleic acids (e.g., mRNA levels). Exemplary methods for processing the sample are described in further detail below.

Therefore, expression levels of soluble mediators in a subject (e.g., SLE patient) can be assessed by obtaining a sample from the subject and by performing an assay to determine the expression levels of the soluble mediators. In another aspect, obtaining a dataset refers to receiving the dataset of expression levels of soluble mediators (e.g., from a third party who has processed the sample (e.g., performed an assay) to determine the expression levels of the soluble mediators).

Soluble Mediators for Predicting a SLE Disease Activity Event

Embodiments described herein use expression levels of one or more soluble mediators for predicting a SLE disease activity event.

In some embodiments, the soluble mediators comprise at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin.

In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), the at least two TNFR superfamily member molecules comprise tumor necrosis factor receptor I (TNFRI), tumor necrosis factor receptor II (TNFRII), the at least two regulatory mediator molecules comprise native transforming growth factor beta (native TGF-β) and an interleukin-1 receptor antagonist (IL-1RA), and the at least one SLE mediator molecule comprises stem cell factor (SCF). In some embodiments, the biomarkers further comprise at least one T-helper type-1 (Th1) cytokines selected from interferon-gamma (IFN-γ), IL-12p70, IL-2, and IL-2R_{α}. In some embodiments, the at least one Th1 cytokine comprises interferon-gamma (IFN-γ).

In some embodiments, the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1); the at least two TNFR superfamily member molecules comprise tumor necrosis factor receptor I (TNFRI), tumor necrosis factor receptor II (TNFRII), and tumor necrosis factor-related apoptosis-inducing ligand (TRAIL), the at least two regulatory mediator molecules comprise native transforming growth factor beta (native TGF-β), an interleukin-1 receptor antagonist (IL-1RA), a total transforming growth factor beta (total TGF-β), and the at least one SLE mediator molecule comprises stem cell factor (SCF), and wherein the biomarkers further comprise one or more T-helper type-1 (Th1) cytokines, wherein the one or more Th1 cytokines comprise an interferon-gamma (IFN-γ).

In some embodiments, the biomarkers comprise: at least one innate cytokine selected from IL-7, IL-1α, and IL-1β; at least one Th1 cytokine selected from interferon-gamma (IFN-γ), IL-12p70, IL-2, and IL-2R_{α}, at least one Th2 cytokine selected from IL-4 and IL-13; at least one Th17 cytokine selected from IL-17A, IL-6, IL-21, and IL-23; at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α). C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8; at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α; at least two regulatory mediator molecules selected from native transforming growth factor beta (native TGF-β), an interleukin-1 receptor antagonist (IL-1RA), a total transforming growth factor beta (total TGF-β), and IL-10; and at least one SLE mediator molecule selected from a stem cell factor (SCF) and Resistin.

In some embodiments, the biomarkers comprise: (i) chemokine(s) or adhesion molecules, wherein the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), and a C—X—C motif chemokine ligand 9 (CXCL9)/monokine induced by interferon-gamma (MIG); (ii) tumor necrosis factor receptor (TNFR) superfamily member molecules, wherein the TNFR superfamily member molecules comprise: a tumor necrosis factor receptor I (TNFRI), and a tumor necrosis factor receptor II (TNFRII); (iii) regulatory mediator molecules, wherein the regulatory mediator molecules comprise: native transforming growth factor beta (native TGF-β), and an interleukin-1 receptor antagonist (IL-1RA); and (iv) one or more systemic lupus erythematosus (SLE) mediator molecules, wherein the one or more SLE mediator molecules comprise a stem cell factor (SCF);

Innate cytokines. Innate cytokines are mediators secreted in response to immune system danger signals, such as toll like receptors (TLR). Innate cytokines which activate and are secreted by multiple immune cell types include Type I interferons (IFN-α and IFN-β), TNF-α, and members of the IL-1 family (IL-1α and IL-1β). Innate soluble mediators in the IL-1 family of pro-inflammatory cytokines also aid in driving the adaptive immune response, including Th17-type differentiation. Other innate cytokines, secreted by antigen presenting cells (APC), including dendritic cells, macrophages, and B-cells, as they process and present protein fragments (antigens, either from infectious agents or self proteins that drive autoimmune disease) to CD4 T-helper (Th) cells, drive the development of antigen specific inflammatory pathways during the adaptive response, described below.

Th1-type cytokines. Th1-type cytokines drive proinflammatory responses responsible for killing intracellular parasites and for perpetuating autoimmune responses. Excessive proinflammatory responses can lead to uncontrolled tissue damage, particularly in systemic lupus erythematosus (SLE).

CD4 Th cells differentiate to Th-1 type cells upon engagement of APC, co-stimulatory molecules, and APC-secreted cytokines, the hallmark of which is IL-12. IL-12 is composed of a bundle of four alpha helices. It is a heterodimeric cytokine encoded by two separate genes, IL-12A (p35) and IL-12B (p40). The active heterodimer, and a homodimer of p40, are formed following protein synthesis. IL-12 binds to the heterodimeric receptor formed by IL-12R-β1 and IL-12R-β2. IL-12R-β2 is considered to play a key role in IL-12 function, as it is found on activated T cells and is stimulated by cytokines that promote Th1 cell development and inhibited by those that promote Th2 cell development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells. IL-12 mediated signaling results in the production of interferon-gamma (IFN-γ) and tumor necrosis factor-alpha (TNF-α) from T and natural killer (NK) cells, and reduces IL-4 mediated suppression of IFN-γ.

IFNγ, or type II interferon, consists of a core of six α-helices and an extended unfolded sequence in the C-terminal region. IFNγ is critical for innate (NK cell) and adaptive (T cell) immunity against viral (CD8 responses) and intracellular bacterial (CD4 Th1 responses) infections and for tumor control. During the effector phase of the immune response, IFNγ activates macrophages. Aberrant IFNγ expression is associated with a number of autoinflammatory and autoimmune diseases, including increased disease activity in SLE.

Although IFNγ is considered to be the characteristic Th1 cytokine, in humans, interleukin-2 (IL-2) has been shown to influence Th1 differentiation, as well as its role as the predominant cytokine secreted during a primary response by naïve Th cells. IL-2 is necessary for the growth, proliferation, and differentiation of T cells to become ‘effector’ T cells. IL-2 is normally produced by T cells during an immune response. Antigen binding to the T cell receptor (TCR) stimulates the secretion of IL-2, and the expression of IL-2 receptors IL-2R. The IL-2/IL-2R interaction then stimulates the growth, differentiation and survival of antigen-specific CD4+ T cells and CD8+ T cells As such, IL-2 is necessary for the development of T cell immunologic memory, which depends upon the expansion of the number and function of antigen-selected T cell clones. IL-2, along with IL-7 and IL-15 (all members of the common cytokine receptor gamma-chain family), maintain lymphoid homeostasis to ensure a consistent number of lymphocytes during cellular turnover.

IL-12p70. CD4 Th cells differentiate to Th-1 type cells upon engagement of APC, co-stimulatory molecules, and APC-secreted cytokines, the hallmark of which is IL-12. IL-12 is composed of a bundle of four alpha helices. It is a heterodimeric cytokine encoded by two separate genes, IL-12A (p35) and IL-12B (p40). The active heterodimer, and a homodimer of p40, are 20 formed following protein synthesis. IL-12 binds to the heterodimeric receptor formed by IL-12R-β1 and IL-12R-β2. IL-12R-β2 is considered to play a key role in IL-12 function, as it is found on activated T cells and is stimulated by cytokines that promote Th1 cell development and inhibited by those that promote Th2 cell development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells. IL-12 mediated signaling results in the production of interferon-gamma (IFN-γ) and tumor necrosis factor-alpha (TNF-α) from T and natural killer (NK) cells, and reduces IL-4 mediated suppression of IFN-γ.

Th2-type cytokines. Th2-type cytokines include IL-4, IL-5, IL-13, as well as IL-6 (in humans), and are associated with the promotion of B-lymphocyte activation, antibody production, and isotype switching to IgE and eosinophilic responses in atopy. In excess, Th2 responses counteract the Th1 mediated microbicidal action. Th2-type cytokines may also contribute to SLE pathogenesis and increased disease activity.

IL-4 is a 15-kD polypeptide with multiple effects on many cell types. Its receptor is a heterodimer composed of an a subunit, with IL-4 binding affinity, and the common 7 subunit which is also part of other cytokine receptors. In T cells, binding of IL-4 to its receptor induces proliferation and differentiation into Th2 cells. IL-4 also contributes to the Th2-mediated activation of B-lymphocytes, antibody production, and, along with IL-5 and IL-13, isotype switching away from Th1-type isotypes (including IgG1 and IgG2) toward Th2-type isotypes (including IgG4, and IgE that contributes to atopy). In addition to its contributions to Th2 biology, IL-4 plays a significant role in immune cell hematopoiesis, with multiple effects on hematopoietic progenitors, including proliferation and differentiation of committed as well as primitive hematopoietic progenitors. It acts synergistically with granulocyte-colony stimulating factor (G-CSF) to support neutrophil colony formation, and, along with IL-1 and IL-6, induces the colony formation of human bone marrow B lineage cells.

IL-5 is an interleukin produced by multiple cell types, including Th2 cells, mast cells, and eosinophils. IL-5 expression is regulated by several transcription factors including GATA3. IL-5 is a 115-amino acid (in human; 133 in the mouse)-long TH2 cytokine that is part of the hematopoietic family. Unlike other members of this cytokine family (namely IL-3 and GM-CSF), this glycoprotein in its active form is a homodimer. Through binding to the IL-5 receptor, IL-5 stimulates B cell growth and increases immunoglobulin secretion. IL-5 has long been associated with the cause of several allergic diseases including allergic rhinitis and asthma, where mast cells play a significant role, and a large increase in the number of circulating, airway tissue, and induced sputum eosinophils have been observed.

Given the high concordance of eosinophils and, in particular, allergic asthma pathology, it has been widely speculated that eosinophils have an important role in the pathology of this disease. IL-13 is secreted by many cell types, but especially Th2 cells as a mediator of allergic inflammation and autoimmune disease, including type 1 diabetes mellitus, rheumatoid arthritis (RA) and SLE. IL-13 induces its effects through a multi-subunit receptor that includes the alpha chain of the IL-4 receptor (IL-4Rα) and at least one of two known IL-13-specific binding chains. Most of the biological effects of IL-13, like those of IL-4, are linked to a single transcription factor, signal transducer and activator of transcription 6 (STAT6).

Like IL-4, IL-13 is known to induce changes in hematopoietic cells, but to a lesser degree. IL-13 can induce immuoglobulin E (IgE) secretion from activated human B cells. IL-13 induces many features of allergic lung disease, including airway hyperresponsiveness, goblet cell metaplasia and mucus hypersecretion, which all contribute to airway obstruction. IL-4 contributes to these physiologic changes, but to a lesser extent than IL-13. IL-13 also induces secretion of chemokines that are required for recruitment of allergic effector cells to the lung.

IL-13 may antagonize Th1 responses that are required to resolve intracellular infections and induces physiological changes in parasitized organs that are required to expel the offending organisms or their products. For example, expulsion from the gut of a variety of mouse helminths requires IL-13 secreted by Th2 cells. IL-13 induces several changes in the gut that create an environment hostile to the parasite, including enhanced contractions and glycoprotein hyper-secretion from gut epithelial cells, that ultimately lead to detachment of the organism from the gut wall and their removal.

Interleukin 6 (IL-6) is secreted by multiple cell types and participates in multiple innate and adaptive immune response pathways. IL-6 mediates its biological functions through a signal-transducing component of the IL-6 receptor (IL-6R), gp130, that leads to tyrosine kinase phosphorylation and downstream signaling events, including the STAT1/3 and the SHP2/ERK cascades. IL-6 is a key mediator of fever and stimulates an acute phase response during infection and after trauma. It is capable of crossing the blood brain barrier and initiating synthesis of PGE2 in the hypothalamus, thereby changing the body's temperature setpoint. In muscle and fatty tissue, IL-6 stimulates energy mobilization which leads to increased body temperature.

IL-6 can be secreted by multiple immune cells in response to specific microbial molecules, referred to as pathogen associated molecular patterns (PAMPs). These PAMPs bind to highly important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production. As a Th2-type cytokine in humans, IL-6, along with IL-4, IL-5, and IL-13, can influence IgE production and eosinophil airway infiltration in asthma. IL-6 also contributes to Th2-type adaptive immunity against parasitic infections, with particular importance in mast-cell activation that coincides with parasite expulsion.

IL-6 is also a Th17-type cytokine, driving IL-17 production by T-lymphocytes in conjunction with TGF-β. IL-6 sensitizes Th17 cells to IL-23 (produced by APC) and IL-21 (produced by T-lymphocytes to perpetuate the Th17 response. Th17-type responses are described below.

Th17-type cytokines. Th17 cells are a subset of T helper cells are considered developmentally distinct from Th1 and Th2 cells and excessive amounts of the cell are thought to play a key role in autoimmune disease, such as multiple sclerosis (which was previously thought to be caused solely by Th1 cells), psoriasis, autoimmune uveitis, Crohn's disease, type 2 diabetes mellitus, rheumatoid arthritis, and SLE. Th17 are thought to play a role in inflammation and tissue injury in these conditions. In addition to autoimmune pathogenesis, Th17 cells serve a significant function in anti-microbial immunity at epithelial/mucosal barriers. They produce cytokines (such as IL-21 and IL-22) that stimulate epithelial cells to produce anti-microbial proteins for clearance of microbes such as Candida and Staphylococcus species. A lack of Th17 cells may leave the host susceptible to opportunistic infections. In addition to its role in autoimmune disease and infection, the Th17 pathway has also been implicated in asthma, including the recruitment of neutrophils to the site of airway inflammation.

Interleukin 17A (IL-17A), is the founding member of a group of cytokines called the IL-17 family. Known as CTLA8 in rodents, IL-17 shows high homology to viral IL-17 encoded by an open reading frame of the T-lymphotropic rhadinovirus Herpesvirus saimiri. IL-17A is a 155-amino acid protein that is a disulfide-linked, homodimeric, secreted glycoprotein with a molecular mass of 35 kDa. Each subunit of the homodimer is approximately 15-20 kDa. The structure of IL-17A consists of a signal peptide of 23 amino acids followed by a 123-residue chain region characteristic of the IL-17 family. An N-linked glycosylation site on the protein was first identified after purification of the protein revealed two bands, one at 15 KDa and another at 20 KDa. Comparison of different members of the IL-17 family revealed four conserved cysteines that form two disulfide bonds. IL-17A is unique in that it bears no resemblance to other known interleukins. Furthermore, IL-17A bears no resemblance to any other known proteins or structural domains.

The crystal structure of IL-17F, which is 50% homologous to IL-17A, revealed that IL-17F is structurally similar to the cysteine knot family of proteins that includes the neurotrophins. The cysteine knot fold is characterized by two sets of paired β-strands stabilized by three disulfide interactions. However, in contrast to the other cysteine knot proteins, IL-17F lacks the third disulfide bond. Instead, a serine replaces the cysteine at this position. This unique feature is conserved in the other IL-17 family members. IL-17F also dimerizes in a fashion similar to nerve growth factor (NGF) and other neurotrophins.

IL-17A acts as a potent mediator in delayed-type reactions by increasing chemokine production in various tissues to recruit monocytes and neutrophils to the site of inflammation, similar to IFNγ. IL-17A is produced by T-helper cells and is induced by APC production of IL-6 (and TGF-φ and IL-23, resulting in destructive tissue damage in delayed-type reactions. IL-17 as a family functions as a proinflammatory cytokine that responds to the invasion of the immune system by extracellular pathogens and induces destruction of the pathogen's cellular matrix. IL-17 acts synergistically with TNF-α and IL-1. To elicit its functions. IL-17 binds to a type I cell surface receptor called IL-17R of which there are at least three variants IL17RA, IL17RB, and IL17RC.

IL-23 is produced by APC, including dendritic cells, macrophages, and B cells. The IL-23A gene encodes the p19 subunit of the heterodimeric cytokine. IL-23 is composed of this protein and the p40 subunit of IL-12. The receptor of IL-23 is formed by the beta 1 subunit of IL12 (IL12RB1) and an IL23 specific subunit, IL23R. While IL-12 stimulates IFNγ production via STAT4, IL-23 primarily stimulates IL-17 production via STAT3 in conjunction with IL-6 and TGF-β.

IL-21 is expressed in activated human CD4 T cells, most notably Th17 cells and T follicular helper (Tfh) cells. IL-21 is also expressed in NK T cells. IL-21 has potent regulatory effects on cells of the immune system, including natural killer (NK) cells and cytotoxic T cells that can destroy virally infected or cancerous cells. This cytokine induces cell division/proliferation in its target cells.

The IL-21 receptor (IL-21R) is expressed on the surface of T, B and NK cells. Belonging to the common cytokine receptor gamma-chain family, IL-21R requires dimerization with the common gamma chain (γc) in order to bind IL-21. When bound to IL-21, the IL-21 receptor acts through the Jak/STAT pathway, utilizing Jak1 and Jak3 and a STAT3 homodimer to activate its target genes.

IL-21 may be a factor in the control of persistent viral infections. IL-21 (or IL-21R) knock-out mice infected with chronic LCMV (lymphocytic choriomeningitis virus) were not able to overcome chronic infection compared to normal mice. Besides, these mice with impaired IL-21 signaling had more dramatic exhaustion of LCMV-specific CD8+ T cells, suggesting that IL-21 produced by CD4+ T cells is required for sustained CD8+ T cell effector activity and then, for maintaining immunity to resolve persistent viral infection. Thus, IL-21 may contribute to the mechanism by which CD4+ T helper cells orchestrate the immune system response to viral infections.

In addition to promoting Th17 responses that contribute to chronic inflammation and tissue damage in autoimmune disease, IL-21 induces Tfh cell formation within the germinal center and signals directly to germinal center B cells to sustain germinal center formation and its response. IL-21 also induces the differentiation of human naïve and memory B cells into anti-body secreting cells, thought to play a role in autoantibody production in SLE.

Chemokines and Adhesion Molecules. Chemokines and adhesion molecules (in this case, ICAM-1 and E-selectin) serve to coordinate cellular traffic within the immune response. Chemokines are divided into CXC (R)eceptor/CXC (L)igand and CCR/CCL subgroups.

Interleukin 8 (IL-8)/CXCL8 is a chemokine produced by macrophages and other cell types such as epithelial cells, airway smooth muscle cells and endothelial cells. In humans, the interleukin-8 protein is encoded by the IL8 gene. IL-8 is a member of the CXC chemokine family. The genes encoding this and the other ten members of the CXC chemokine family form a cluster in a region mapped to chromosome 4q.

There are many receptors of the surface membrane capable to bind IL-8: the most frequently studied types are the G protein-coupled serpentine receptors CXCR1, and CXCR2, expressed by neutrophils and monocytes. Expression and affinity to IL-8 is different in the two receptors (CXCR1>CXCR2). IL-8 is secreted and is an important mediator of the immune reaction in the innate immunity in response to TLR engagement. During the adaptive immune response, IL-8 is produced during the effector phase of Th1 and Th17 pathways, resulting in neutrophil and macrophage recruitment to sites of inflammation, including inflammation during infection and autoimmune disease. While neutrophil granulocytes are the primary target cells of IL-8, there are a relative wide range of cells (endothelial cells, macrophages, mast cells, and keratinocytes) also responding to this chemokine.

Monokine induced by γ-interferon (MIG)/CXCL9 is a T-cell chemoattractant induced by IFN-γ. It is closely related to two other CXC chemokines, IP-10/CXCL10 and I-TAC/CXCL11, whose genes are located near the CXCL9 gene on human chromosome 4. MIG, IP-10, and I-TAC elicit their chemotactic functions by interacting with the chemokine receptor CXCR3.

Interferon gamma-induced protein 10 (IP-10), also known as CXCL10, or small-inducible cytokine B10, is an 8.7 kDa protein that in humans is encoded by the CXCL10 gene located on human chromosome 4 in a cluster among several other CXC chemokines. IP-10 is secreted by several cell types in response to IFN-γ. These cell types include monocytes, endothelial cells and fibroblasts. IP-10 has been attributed to several roles, such as chemoattraction for monocytes/macrophages, T cells, NK cells, and dendritic cells, promotion of T cell adhesion to endothelial cells, antitumor activity, and inhibition of bone marrow colony formation and angiogenesis. This chemokine elicits its effects by binding to the cell surface chemokine receptor CXCR3, which can be found on both Th1 and Th2 cells.

Monocyte chemotactic protein-1 (MCP-1)/CCL2 recruits monocytes, memory T cells, and dendritic cells to sites of inflammation. MCP-1 is a monomeric polypeptide, with a molecular weight of approximately 13 kDa that is primarily secreted by monocytes, macrophages and dendritic cells. Platelet derived growth factor is a major inducer of MCP-1 gene. The MCP-1 protein is activated post-cleavage by metalloproteinase MMP-12. CCR2 and CCR4 are two cell surface receptors that bind MCP-1. During the adaptive immune response, CCR2 is upregulated on Th17 and T-regulatory cells, while CCR4 is upregulated on Th2 cells. MCP-1 is implicated in pathogeneses of several diseases characterized by monocytic infiltrates, such as psoriasis, rheumatoid arthritis and atherosclerosis. It is also implicated in the pathogenesis of SLE and a polymorphism of MCP-1 is linked to SLE in Caucasians. Administration of anti-MCP-1 antibodies in a model of glomerulonephritis reduces infiltration of macrophages and T cells, reduces crescent formation, as well as scarring and renal impairment.

Monocyte-specific chemokine 3 (MCP-3)/CCL7) specifically attracts monocytes and regulates macrophage function. It is produced by multiple cell types, including monocytes, macrophages, and dendritic cells. The CCL7 gene is located on chromosome 17 in humans, in a large cluster containing other CC chemokines. MCP-3 is most closely related to MCP-1, binding to CCR2.

Macrophage inflammatory protein-1α (MIP-1α)/CCL3 is encoded by the CCL3 gene in humans. MIP-1α is involved in the acute inflammatory state in the recruitment and activation of polymorphonuclear leukocytes (Wolpe et al., 1988). MIP-1α interacts with MIP-1β/CCL4, encoded by the CCL4 gene, with specificity for CCR5 receptors. It is a chemoattractant for natural killer cells, monocytes and a variety of other immune cells.

Soluble cell adhesion molecules (sCAMs) are a class of cell surface binding proteins that may represent important biomarkers for inflammatory processes involving activation or damage to cells such as platelets and the endothelium. They include soluble forms of the cell adhesion molecules ICAM-1, VCAM-1, E-selectin, L-selectin, and P-selectin (distinguished as sICAM-1, sVCAM-1, sE-selectin, sL-selectin, and sP-selectin). The cellular expression of CAMs is difficult to assess clinically, but these soluble forms are present in the circulation and may serve as markers for CAMs.

ICAM-1 (Intercellular Adhesion Molecule 1) also known as CD54, is encoded by the ICAM gene in humans. This gene encodes a cell surface glycoprotein which is typically expressed on endothelial cells and cells of the immune system. The protein encoded by this gene is a type of intercellular adhesion molecule continuously present in low concentrations in the membranes of leukocytes and endothelial cells. ICAM-1 can be induced by IL-1 and TNF-α, and is expressed by the vascular endothelium, macrophages, and lymphocytes.

The presence of heavy glycosylation and other structural characteristics of ICAM-1 lend the protein binding sites for numerous ligands. ICAM-1 possesses binding sites for a number of immune-associated ligands. Notably, ICAM-1 binds to macrophage adhesion ligand-1 (Mac-1; ITGB2/ITGAM), leukocyte function associated antigen-1 (LFA-1), and fibrinogen. These three proteins are generally expressed on endothelial cells and leukocytes, and they bind to ICAM-1 to facilitate transmigration of leukocytes across vascular endothelia in processes such as extravasation and the inflammatory response. As a result of these binding characteristics, ICAM-1 has classically been assigned the function of intercellular adhesion.

ICAM-1 is a member of the immunoglobulin superfamily, the superfamily of proteins, including B-cell receptors (membrane-bound antibodies) and T-cell receptors. In addition to its roles as an adhesion molecule, ICAM-1 has been shown to be a co-stimulatory molecule for the TCR on T-lymphocytes. The signal-transducing functions of ICAM-1 are associated primarily with proinflammatory pathways. In particular, ICAM-1 signaling leads to recruitment of inflammatory immune cells such as macrophages and granulocytes.

Different from P-selectin, which is stored in vesicles called Weibel-Palade bodies, E-selectin is not stored in the cell and has to be transcribed, translated, and transported to the cell surface. The production of E-selectin is stimulated by the expression of P-selectin which is stimulated by TNF-α. IL-1 and through engagement of TLR4 by LPS. It takes about two hours, after cytokine recognition, for E-selectin to be expressed on the endothelial cell's surface. Maximal expression of E-selectin occurs around 6-12 hours after cytokine stimulation, and levels returns to baseline within 24 hours.

E-selectin recognizes and binds to sialylated carbohydrates present on the surface proteins of leukocytes. E-selectin ligands are expressed by neutrophils, monocytes, eosinophils, memory-effector T-like lymphocytes, and natural killer cells. Each of these cell types is found in acute and chronic inflammatory sites in association with expression of E-selectin, thus implicating E-selectin in the recruitment of these cells to such inflammatory sites. These carbohydrates include members of the Lewis X and Lewis A families found on monocytes, granulocytes, and T-lymphocytes.

TNF Receptor superfamily members. The tumor necrosis factor receptor (TNFR) superfamily of receptors and their respective ligands activate signaling pathways for cell survival, death, and differentiation. Members of the TNFR superfamily act through ligand-mediated trimerization and require adaptor molecules (e.g. TRAFs) to activate downstream mediators of cellular activation, including NF-κB and MAPK pathways, immune and inflammatory responses, and in some cases, apoptosis.

The prototypical member is TNF-α. Tumor necrosis factor (TNF, cachexin, or cachectin, and formerly known as tumor necrosis factor alpha or TNFα) is a cytokine involved in systemic inflammation and is a member of a group of cytokines that stimulate the acute phase reaction. It is produced by a number of immune cells, including macrophages, dendritic cells, and both T- and B-lymphocytes. Dysregulation of TNF-α production has been implicated in a variety of human diseases including Alzheimer's disease, cancer, major depression and autoimmune disease, including inflammatory bowel disease (IBD) and rheumatoid arthritis (RA).

TNF-α is produced as a 212-amino acid-long type II transmembrane protein arranged in stable homotrimers. From this membrane-integrated form the soluble homotrimeric cytokine (sTNF) is released via proteolytic cleavage by the metalloprotease TNF-α converting enzyme (TACE, also called ADAM17). The soluble 51 kDa trimeric sTNF may dissociate to the 17-kD monomeric form. Both the secreted and the membrane bound forms are biologically active. Tumor necrosis factor receptor 1 (TNFRI; TNFRSF1a; CD120a), is a trimeric cytokine receptor that is expressed in most tissues and binds both membranous and soluble TNF-α. The receptor cooperates with adaptor molecules (such as TRADD, TRAF, RIP), which is important in determining the outcome of the response (e.g., apoptosis, inflammation). Tumor necrosis factor II (TNFRII; TNFRSF1b; CD120b) has limited expression, primarily on immune cells (although during chronic inflammation, endothelial cells, including those of the lung and kidney, are induced to express TNFRII) and binds the membrane-bound form of the TNF-α homotrimer with greater affinity and avidity than soluble TNF-α. Unlike TNFRI, TNFRII does not contain a death domain (DD) and does not cause apoptosis, but rather contributes to the inflammatory response and acts as a co-stimulatory molecule in receptor-mediated B- and T-lymphocyte activation.

Fas, also known as apoptosis antigen 1 (APO-1 or APT), cluster of differentiation 95 (CD95) or tumor necrosis factor receptor superfamily member 6 (TNFRSF6) is a protein that in humans is encoded by the TNFRSF6 gene located on chromosome 10 in humans and 19 in mice. Fas is a death receptor on the surface of cells that leads to programmed cell death (apoptosis). Like other TNFR superfamily members, Fas is produced in membrane-bound form, but can be produced in soluble form, either via proteolytic cleavage or alternative splicing. The mature Fas protein has 319 amino acids, has a predicted molecular weight of 48 kD and is divided into 3 domains: an extracellular domain, a transmembrane domain, and a cytoplasmic domain. Fas forms the death-inducing signaling complex (DISC) upon ligand binding. Membrane-anchored Fas ligand on the surface of an adjacent cell causes oligomerization of Fas. Upon ensuing death domain (DD) aggregation, the receptor complex is internalized via the cellular endosomal machinery. This allows the adaptor molecule FADD to bind the death domain of Fas through its own death domain.

In most cell types, caspase-8 catalyzes the cleavage of the pro-apoptotic BH3-only protein Bid into its truncated form, tBid. BH-3 only members of the Bcl-2 family exclusively engage anti-apoptotic members of the family (Bcl-2, Bcl-xL), allowing Bak and Bax to translocate to the outer mitochondrial membrane, thus permeabilizing it and facilitating release of pro-apoptotic proteins such as cytochrome c and Smac/DIABLO, an antagonist of inhibitors of apoptosis proteins (IAPs).

Fas ligand (FasL; CD95L; TNFSF6) is a type-II transmembrane protein that belongs to the tumor necrosis factor (TNF) family. Its binding with its receptor induces apoptosis. FasL/Fas interactions play an important role in the regulation of the immune system and the progression of cancer. Soluble Fas ligand is generated by cleaving membrane-bound FasL at a conserved cleavage site by the external matrix metalloproteinase MMP-7.

Apoptosis triggered by Fas-Fas ligand binding plays a fundamental role in the regulation of the immune system. Its functions include T-cell homeostasis (the activation of T-cells leads to their expression of the Fas ligand. T cells are initially resistant to Fas-mediated apoptosis during clonal expansion, but become progressively more sensitive the longer they are activated, ultimately resulting in activation-induced cell death (AICD)), cytotoxic T-cell activity (Fas-induced apoptosis and the perforin pathway are the two main mechanisms by which cytotoxic T lymphocytes induce cell death in cells expressing foreign antigens), immune privilege (cells in immune privileged areas such as the cornea or testes express Fas ligand and induce the apoptosis of infiltrating lymphocytes), maternal tolerance (Fas ligand may be instrumental in the prevention of leukocyte trafficking between the mother and the fetus, although no pregnancy defects have yet been attributed to a faulty Fas-Fas ligand system) and tumor counterattack (tumors may over-express Fas ligand and induce the apoptosis of infiltrating lymphocytes, allowing the tumor to escape the effects of an immune response).

CD154, also called CD40 ligand (CD40L), is a member of the TNF superfamily protein that is expressed primarily on activated T cells. CD40L binds to CD40 (TNFRSF4), which is constitutively expressed by antigen-presenting cells (APC), including dendritic cells, macrophages, and B cells. CD40L engagement of CD40 induces maturation and activation of dendritic cells and macrophages in association with T cell receptor stimulation by MHC molecules on the APC. CD40L regulates B cell activation, proliferation, antibody production, and isotype switching by engaging CD40 on the B cell surface. A defect in this gene results in an inability to undergo immunoglobulin class switch and is associated with hyper IgM syndrome. While CD40L was originally described on T lymphocytes, its expression has since been found on a wide variety of cells, including platelets, endothelial cells, and aberrantly on B lymphocytes during periods of chronic inflammation.

TNF Related Apoptosis Inducing Ligand (TRAIL) is apart of the TNF superfamily. TNF superfamily member (TNFS10) mediates apoptosis in sensitive cells and contributes to the pro-inflammatory response when engaging its receptor, TRAIL-R.

NGF-beta is a member of the nerve growth factor family of molecules, related to the TNF superfamily mediators/receptors. It has context-dependent, pro- and anti-inflammatory properties. In addition to nerve cell activation, NGF-beta plays a role in immune cell-mediated inflammation.

Other Soluble Mediators. Stem Cell Factor (also known as SCF, kit-ligand, KL, or steel factor) is a cytokine that binds to the c-Kit receptor (CD117). SCF is categorized as a SLE mediator molecule. SCF can exist both as a transmembrane protein and a soluble protein. This cytokine plays an important role in hematopoiesis (formation of blood cells), spermatogenesis, and melanogenesis. The gene encoding stem cell factor (SCF) is found on the SI locus in mice and on chromosome 12q22-12q24 in humans. The soluble and transmembrane forms of the protein are formed by alternative splicing of the same RNA transcript.

The soluble form of SCF contains a proteolytic cleavage site in exon 6. Cleavage at this site allows the extracellular portion of the protein to be released. The transmembrane form of SCF is formed by alternative splicing that excludes exon 6. Both forms of SCF bind to c-Kit and are biologically active. Soluble and transmembrane SCF is produced by fibroblasts and endothelial cells. Soluble SCF has a molecular weight of 18.5 kDa and forms a dimer. SCF plays an important role in the hematopoiesis, providing guidance cues that direct hematopoietic stem cells (HSCs) to their stem cell niche (the microenvironment in which a stem cell resides), and it plays an important role in HSC maintenance. SCF plays a role in the regulation of HSCs in the stem cell niche in the bone marrow. SCF has been shown to increase the survival of HSCs in vitro and contributes to the self-renewal and maintenance of HSCs in vivo. HSCs at all stages of development express the same levels of the receptor for SCF (c-Kit). The stromal cells that surround HSCs are a component of the stem cell niche, and they release a number of ligands, including SCF.

A small percentage of HSCs regularly leave the bone marrow to enter circulation and then return to their niche in the bone marrow. It is believed that concentration gradients of SCF, along with the chemokine SDF-1, allow HSCs to find their way back to the niche.

In addition to hematopoiesis, SCF is thought to contribute to inflammation via its binding to c-kit on dendritic cells. This engagement leads to increased secretion of IL-6 and the promoted development of Th2 and Th17-type immune responses. Th2 cytokines synergize with SCF in the activation of mast cells, and integral promoter of allergic inflammation. The induction of IL-17 allows for further upregulation of SCF by epithelial cells and the promotion of granulopoiesis. In the lung, the upregulation of IL-17 induces IL-8 and MIP-2 to recruit neutrophils to the lung. The chronic induction of IL-17 has been demonstrated to play a role in autoimmune diseases, including multiple sclerosis and rheumatoid arthritis.

Resistin. Resistin, categorized as a SLE mediator molecule, is a pro-inflammatory molecule produced in white adipose tissue that contributes to insulin resistance and is associated with organ damage/dysfunction, including renal dysfunction.

IL-10. Interleukin-10 (IL-10), also known as human cytokine synthesis inhibitory factor (CSIF), is an anti-inflammatory cytokine. The IL-10 protein is a homodimer; each of its subunits is 178-amino-acid long. IL-10 is classified as a class-2 cytokine, a set of cytokines including IL-19, IL-20, IL-22, IL-24 (Mda-7), and IL-26, interferons and interferon-like molecules. In humans, IL-10 is encoded by the IO gene, which is located on chromosome 1 and comprises 5 exons. IL-10 is primarily produced by monocytes and lymphocytes, namely Th2 cells, CD4⁺CD25⁺Foxp3⁺ regulatory T cells, and in a certain subset of activated T cells and B cells. IL-10 can be produced by monocytes upon PD-1 triggering in these cells. The expression of IL-10 is minimal in unstimulated tissues and requires receptor-mediated cellular activation for its expression. IL-10 expression is tightly regulated at the transcriptional and post-transcriptional level. Extensive IL-10 locus remodeling is observed in monocytes upon stimulation of TLR or Fc receptor pathways. IL-10 induction involves ERK1/2, p38 and NFκB signaling and transcriptional activation via promoter binding of the transcription factors NFκB and AP-1. IL-10 may autoregulate its expression via a negative feed-back loop involving autocrine stimulation of the IL-10 receptor and inhibition of the p38 signaling pathway. Additionally, IL-10 expression is extensively regulated at the post-transcriptional level, which may involve control of mRNA stability via AU-rich elements and by microRNAs such as let-7 or miR-106.

IL-10 is a cytokine with pleiotropic effects in immunoregulation and inflammation. It downregulates the expression of multiple Th-pathway cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway.

TGF-β. Transforming growth factor beta (TGF-β) controls proliferation, cellular differentiation, and other functions in most cells. TGF-β is a secreted protein that exists in at least three isoforms called TGF-β1, TGF-β2 and TGF-β3. It was also the original name for TGF-β1, which was the founding member of this family. The TGF-β family is part of a superfamily of proteins known as the transforming growth factor beta superfamily, which includes inhibins, activin, anti-mullerian hormone, bone morphogenetic protein, decapentaplegic and Vg-1.

Most tissues have high expression of the gene encoding TGF-β. That contrasts with other anti-inflammatory cytokines such as IL-10, whose expression is minimal in unstimulated tissues and seems to require triggering by commensal or pathogenic flora.

The peptide structures of the three members of the TGF-β family are highly similar. They are all encoded as large protein precursors; TGF-β1 contains 390 amino acids and TGF-β2 and TGF-β3 each contain 412 amino acids. They each have an N-terminal signal peptide of 20-30 amino acids that they require for secretion from a cell, a pro-region (called latency associated peptide or LAP), and a 112-114 amino acid C-terminal region that becomes the mature TGF-β molecule following its release from the pro-region by proteolytic cleavage. The mature TGF-β protein dimerizes to produce a 25 kDa active molecule with many conserved structural motifs.

TGF-β plays a crucial role in the regulation of the cell cycle. TGF-β causes synthesis of p15 and p21 proteins, which block the cyclin:CDK complex responsible for Retinoblastoma protein (Rb) phosphorylation. Thus TGF-β blocks advance through the G1 phase of the cycle TGF-β is necessary for CD4⁺CD25⁺Foxp3⁺ T-regulatory cell differentiation and suppressive function. In the presence of IL-6, TGF-β contributes to the differentiation of pro-inflammatory Th17 cells.

Two subforms of TGF-β are often detected depending on design of particular immunoassays. Specifically, the TGF-β latent or TGF-β total (name interchangeable depending on vendor and research investigator preference) form of TGF-beta1 is comprised of a complex between TGF-beta1 (native/active form) and latency associated peptide (LAP). Additionally, the TGF-β native or TGF-β active (name interchangeable depending on vendor and research investigator preference) form of TGF-beta1 is the biologically active form whereby LAP has been dissociated. Some antibody (Ab) pairs in immunoassays pick up the latent (“Total”) form, while others pick up the native/active form. Both forms are biologically informative in determining risk of imminent clinical disease flare in SLE patients.

SDF-1. Stromal cell-derived factor 1 (SDF-1), also known as C—X—C motif chemokine 12 (CXCL12), is encoded by the CXCL12 gene on chromosome 10 in humans. SDF-1 is produced in two forms, SDF-1α/CXCL12a and SDF-1β/CXCL12b, by alternate splicing of the same gene. Chemokines are characterized by the presence of four conserved cysteines, which form two disulfide bonds. The CXCL12 proteins belong to the group of CXC chemokines, whose initial pair of cysteines are separated by one intervening amino acid.

CXCL12 is strongly chemotactic for lymphocytes. During embryogenesis it directs the migration of hematopoietic cells from fetal liver to bone marrow and the formation of large blood vessels. CXCL12 knockout mice are embryonic lethal.

The receptor for this chemokine is CXCR4, which was previously called LESTR or fusin. This CXCL12-CXCR4 interaction was initially thought to be exclusive (unlike for other chemokines and their receptors), but recently it was suggested that CXCL12 may also bind the CXCR7 receptor. The CXCR4 receptor is a G-Protein Coupled Receptor that is widely expressed, including on T-regulatory cells, allowing them to be recruited to promote lymphocyte homeostasis and immune tolerance. In addition to CXCL12, CXCR4 binds Granulocyte-Colony Stimulating Factor (G-CSF). G-CSF binds CXCR4 to prevent SDF-1 binding, which results in the inhibition of the pathway.

IL-1RA. The interleukin-1 receptor antagonist (IL-1RA) is a protein that in humans is encoded by the IL1RN gene. A member of the IL-1 cytokine family, IL-1RA, is an agent that binds non-productively to the cell surface interleukin-1 receptor (IL-1R), preventing IL-1 from binding and inducing downstream signaling events.

IL1RA is secreted by various types of cells including immune cells, epithelial cells, and adipocytes, and is a natural inhibitor of the pro-inflammatory effect of IL-1α and IL1β. This gene and five other closely related cytokine genes form a gene cluster spanning approximately 400 kb on chromosome 2. Four alternatively spliced transcript variants encoding distinct isoforms have been reported.

An interleukin 1 receptor antagonist is used in the treatment of rheumatoid arthritis, an autoimmune disease in which IL-1 plays a key role. It is commercially produced as anakinra, which is a human recombinant form of IL-1RA Anakinra has shown both safety and efficacy in improving arthritis in an open trial on four SLE patients, with only short-lasting therapeutic effects in two patients.

UniProt identifiers for the biomarkers disclosed herein are shown in Table 4.

Assessing Soluble Mediator Expression

In accordance with the present invention, methods are provided for assessing the expression levels of soluble mediators. In each of these assays, the expression of various soluble mediators will be measured, and in some, the expression is measured multiple times to assess not only absolute values, but changes in these values overtime. Virtually any method of measuring gene expression may be utilized, and the following discussion is exemplary in nature and in no way limiting.

Immunologic Assays

There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (see, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In accordance with the present invention, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev O, 1999; Gulbis and Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section. ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, this process entails coating antibody specific for the protein of interest is placed in each well of a 96-well plate. After appropriate incubation, wash, and plate blocking (nonspecific protein to coat wells and eliminate false positive detection), samples and recombinant protein standards (for quantification) are loaded on to the plate. After appropriate incubation and plate washing, a second, biotinylated detection antibody, specific for the protein of interest, but for a different physical part of the protein (epitope) from the coating antibody, is loaded into the well. After appropriate incubation and plate washing, a streptavidin tagged enzyme is loaded onto the plate. After appropriate incubation and plate washing, an appropriate substrate for the enzyme is plated and color change noted. After appropriate color development (based on the standard curve [positive] and blank [negative] wells), the reaction is stopped with an appropriate reagent (acid or buffer) that both stops the reaction and changes the color of the reaction (e.g. from blue to yellow). The assay plate is then read on a 96-well spectrophotometer, blank subtraction performed, a 5-parameter standard curve rendered, and interpolation of the sample data to render concentration of the protein of interest detected within the given samples. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/TWEEN® (polysorbate 20). These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Another antibody-based approach to assessing biomarkers expression is Fluorescence-Activated Cell Sorting (FACS), a specialized type of flow cytometry. It provides a method for sorting a heterogeneous mixture of biological cells into two or more containers, one cell at a time, based upon the specific light scattering and fluorescent characteristics of each cell. It provides fast, objective and quantitative recording of fluorescent signals from individual cells as well as physical separation of cells of particular interest. A cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off. One common way to use FAC is with a fluorescently labeled antibody that binds to a target on or in a cell, thereby identifying cells with a given target. This technique can be used quantitatively where the amount of fluorescent activity correlates to the amount of target, thereby permitting one to sort based on relative amounts of fluorescence, and hence relative amounts of the target.

Bead-based xMAP® Technology may also be applied to immunologic detection in conjunction with the presently claimed invention. This technology combines advanced fluidics, optics, and digital signal processing with proprietary microsphere technology to deliver multiplexed assay capabilities. Featuring a flexible, open-architecture design, xMAP® technology can be configured to perform a wide variety of bioassays quickly, cost-effectively and accurately.

Fluorescently-coded microspheres are arranged in up to 500 distinct sets. Each bead set can be coated with a reagent specific to a particular bioassay (e.g., an antibody), allowing the capture and detection of specific analytes from a sample, such as the biomarkers of the present application. The basis of the technology is polystyrene or paramagnetic microspheres that are fluorescence-encoded into multiple, spectrally distinct identifiable sets. Coating antibodies (one specificity/bead address) are conjugated onto the fluorescence-encoded beads and used as a solution matrix for an ELISA-type assay. Similar to the ELISA protocol, sample/standard/blanks are incubated with the antibody-coated fluorescent beads. After appropriate incubation/washing of the beads, a biotinylated detection antibody is incubated with the beads, followed by washing and incubation with streptavidin-phycoerythrin (SAPE). After appropriate incubation time the SAPE is washed off and a reading buffer added to each well of the 96-well plate (specific for fluorescent assays). Many readings are made on each bead set, which further validates the results. Multiple light sources inside the Luminex-based analyzer excite (1) the internal bead dyes that identify each microsphere particle and (2) quantity of signal from ELISA-type reaction, whereby blank subtraction is performed, a 5-parameter standard curve rendered, and interpolation of the sample data to render concentration of the proteins of interest detected within the given samples.

Using this process, xMAP® Technology allows multiplexing of up to 500 unique bioassays within a single sample, both rapidly and precisely. Unlike other flow cytometer microsphere-based assays which use a combination of different sizes and color intensities to identify an individual microsphere, xMAP® technology uses 5.6 micron size microspheres internally dyed with red and infrared fluorophores via a proprietary dying process to create 500 unique dye mixtures which are used to identify each individual microsphere.

Some of the advantages of xMAP® include multiplexing (reduces costs and labor), generation of more data with less sample, less labor and lower costs, faster, more reproducible results than solid, planar arrays, and focused, flexible multiplexing of 1 to 500 analytes to meet a wide variety of applications.

Simple Plex™ assays from ProteinSimple/Bio-techne (San Jose, Calif.) can also be used for detection of expression levels of biomarkers. Assays are performed on the Ella platform, a single- or multi-analyte immunoassay platform that enables simultaneous quantitation of four (single) up to eight (multi) analytes from up to 32 individual samples in a single disposable microfluidic cartridge. This approach takes the sandwich ELISA assay, adds the added fluorescence benefits of the xMAP multiplex assay (enhanced signal, increased flexibility, and decreased user inter-assay variability), and applies nanotechnology (glass nano reactors [GNRs]) to further limit inter-assay variability challenges. Briefly, coating antibodies are conjugated in triplicate within each of four channels of the GNR (one monoclonal antibody per channel for single-analyte assays; two monocolonal antibodies to distinct immune mediators per channel for multi-analyte assays). Appropriately diluted samples (up to 32) are loaded into each cartridge inlet where each sample interacts with its respective, antibody coated, channels in parallel (“parallel-plexing”). After appropriate incubation and washing, the cartridge then releases appropriately paired biotinylated detection antibody into each respective channel, followed by release of streptavidin-conjugated fluorescent dye. Each analyte is measured by fluorescence quantitation of the triplicate GNRs for each channel based on lot-specific, manufacturer-determined blank subtraction and 5-parameter standard curve interpolation.

The advantages of the SimplePlex™ include high sensitivity and specificity, t parallel microfluidics allow for each assay channel to be limited to one or two antibody pair(s), thus eliminating interference issues commonly experienced with xMAP multiplex technology, reduced sampling volumes (average of 25 ul of sample required per cartridge), decreased inter- and intra-lot variations due to fixed nature of standards and reagents by manufacturer (minimize user and protocol-associated variability), and controlled assay temperature which limits daily/site-specific environmental variations.

Assays for Nucleic Acid Detection

In alternative embodiments for detecting expression levels of soluble mediators, one may assay for gene transcription. For example, an indirect method for detecting protein expression is to detect mRNA transcripts from which the proteins are made. Methods for amplifying nucleic acids, detecting nucleic acids, using example nucleic acid arrays and microarrays, and sequencing of nucleic acids for the purposes of determining quantitative expression values is described in further detail in U.S. patent application Ser. No. 15/234,754, which is hereby incorporated by reference in its entirety.

Generation of a Lupus Predictive Flare Index

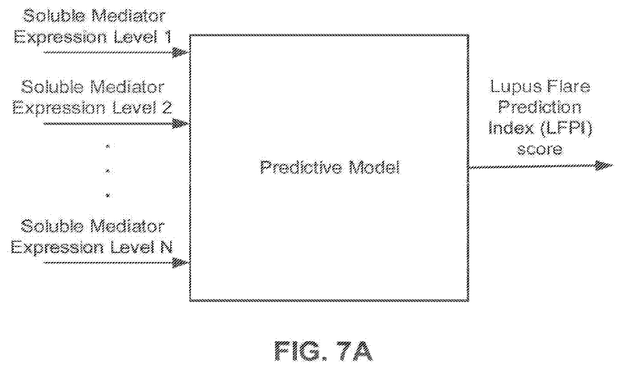

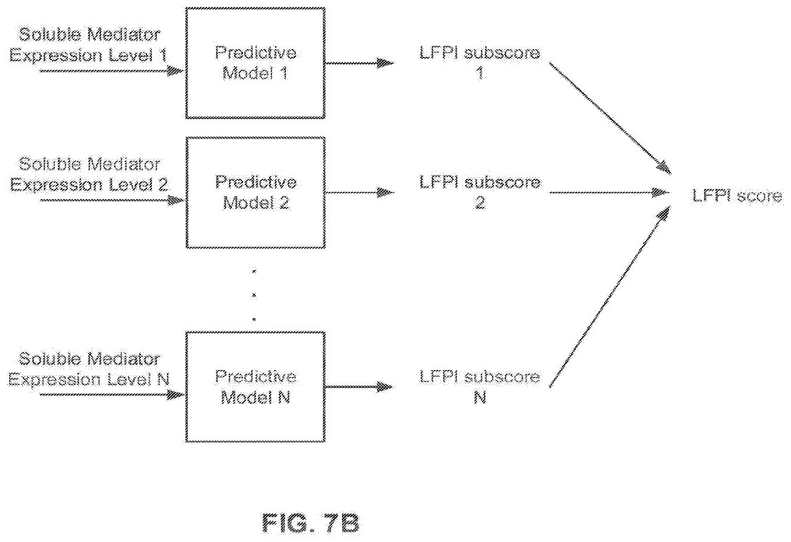

Generally, the soluble mediator expression levels are analyzed using a predictive model to predict the likelihood of a SLE disease activity event (e.g., likelihood of impending flare or likelihood of organ damage). Reference is now made to FIG. 7A which depicts an exemplary embodiment of a predictive model for predicting a score based on soluble mediator expression levels.

Referring to FIG. 7A, in various embodiments, a predictive model predicts a likelihood of a SLE disease activity event through a multivariate analysis of multiple soluble mediator expression levels. Specifically, FIG. 7A depicts a predictive model that is applied to N different soluble mediator expression levels corresponding to N different soluble mediators. The predictive model outputs a LFPI score which represents the likelihood of a SLE disease activity event in the subject based on the multivariate analysis of multiple expression levels of different soluble mediators.

In one embodiment, for each expression level of a soluble mediator, the predictive model transforms the expression level of the soluble mediator to a value that is standardized across various patient samples. In an exemplary embodiment, the predictive model log-transforms the soluble mediator expression level and standardizes the log-transformed value based on an average soluble mediator expression level across a set of SLE patients.

In some embodiments, a set of SLE patients includes SLE patients that, between a baseline clinical visit and a follow-up clinical visit, have experienced a clinical disease flare. In some embodiments, a set of SLE patients includes SLE patients that, between a baseline clinical visit and a follow-up clinical visit, have not experienced a clinical disease flare. In some embodiments, a set of SLE patients includes SLE patients that, over the course of multiple clinic visits, both have experienced a clinical disease flare and have not experienced a clinical disease flare. For example, such SLE patients may not have experienced a clinical disease flare at a first clinic visit, experienced a clinical disease flare at a second clinic visit, and subsequently did not experience a clinical disease flare at third and fourth clinic visits.

In various embodiments, a set of SLE patients includes SLE patients that are categorized according to common characteristics of the SLE patients. Characteristics of the SLE patients can include gender of SLE patients, race/ethnicity of SLE patients (e.g. European American, African American, Native American, Asian, Pacific Islander, Hispanic), age (e.g., ≥18 years of age; 18-100 years of age), and a level of ongoing clinical disease activity (low disease activity, active disease, high clinical disease activity). In some embodiments, a set of SLE patients includes the SLE subject and therefore, the predictive model standardizes the expression value to past data including expression levels of the same SLE subject.

In one embodiment, the predictive model weighs the standardized expression level value using a coefficient obtained from a linear regression that measures the association between pre-flare expression levels of the soluble mediator(s) and a measurement of SLE clinical disease activity at the time of the follow-up visit. The linear regression is described in further detail below. In various embodiments, the predictive model combines the weighted, standardized expression level values across the soluble mediators to generate the LFPI score.

FIG. 7B depicts an embodiment that employs multiple predictive models, each of which is applied to a soluble mediator expression level to generate a LFPI subscore. The LFPI subscores are combined to generate the LFPI score. As shown in FIG. 7B, predictive model 1 can be applied to soluble mediator expression level 1 to generate LFPI subscore 1. Predictive model 2 can be applied to soluble mediator expression level 2 to generate LFPI subscore 2. Predictive model N can be applied to soluble mediator expression level N to generate LFPI subscore N. In various embodiments, each predictive model may perform a subset of steps described above in reference to the predictive model in FIG. 7A. For example, each predictive model in FIG. 7B may transform the expression level of the soluble mediator to a value that is standardized across various patient samples. In one embodiment, the predictive model log-transforms the soluble mediator expression level and standardizes the log-transformed value based on an average soluble mediator expression level across a set of SLE patients. Each predictive model weighs the standardized expression level value using a coefficient obtained from a linear regression that measures the association between pre-flare expression levels of the soluble mediator(s) and a measurement of SLE clinical disease activity at the time of the follow-up visit. Here, the weighted standardized expression level value represents the LFPI subscore for the corresponding soluble mediator.

The LFPI subscores (e.g., LFPI subscore 1, LFPI subscore 2, LFPI subscore N) can be combined to generate the LFPI score that is predictive of whether a subject is likely to experience a SLE disease activity event. In one embodiment, the LFPI subscores are summated to generate the LFPI score. In other embodiments, the LFPI subscores are differently combined (e.g., multiplied or averaged) to generate the LFPI score.

To determine whether a subject is likely to experience a SLE disease activity event (e.g., an impending flare or organ inflammation), the LFPI score for the subject is compared to LFPI scores of prior SLE patients that have either experienced a SLE disease activity event or did not experience a SLE disease activity event. In one embodiment, prior SLE patients refers to a reference set of patients that have experienced a SLE disease activity event. In one embodiment, prior SLE patients refers to a reference set of patients that have not experienced a SLE disease activity event. In one embodiment, the subject is included in the reference set of patients because at a prior timepoint, the subject experienced a SLE disease activity event or did not experience a SLE disease activity event. For example, the LFPI score for the subject is compared to an average of LFPI scores of prior SLE patients that subsequently experienced a flare event and/or the LFPI score for the subject is compared to an average of LFPI scores of prior SLE patients that did not subsequently experience a flare event. Thus, the subject can be classified as either likely to experience a SLE disease activity event or unlikely to experience a SLE disease activity event based on the comparison. For example, if the subject's LFPI score falls within a threshold amount (e.g., standard deviation) of the average LFPI score of prior SLE patients that experienced a flare event, then the subject is classified as likely to experience a flare event. If the subject's LFPI score falls within a threshold amount (e.g., standard deviation) of the average LFPI score of prior SLE patients that did not experience a flare event, then the subject is classified as unlikely to experience a flare event. As another example, if the LFPI score is above a threshold number (e.g., greater than zero), then the subject is classified as likely to experience a flare event. If the LFPI score is below a threshold number (e.g., less than zero), then the subject is classified as unlikely to experience a flare event.

In various embodiments the classification of the SLE subject as likely to experience a SLE disease activity event occurs at least 30 days prior to the SLE subject experiencing a SLE disease activity event. In some embodiments, the classification occurs 60 days, 90 days, 120 days, 150 days, or 180 days prior to the SLE subject experiencing a SLE disease activity event. In some embodiments, the classification occurs between 30 and 150 days prior to the SLE subject experiencing a SLE disease activity event. In some embodiments, the classification occurs between 50 and 120 days prior to the SLE subject experiencing a SLE disease activity event. In some embodiments, the classification occurs between 75 and 100 days prior to the SLE subject experiencing a SLE disease activity event.

If the SLE subject is classified as likely to experience a SLE disease activity event, a treatment can be provided to the SLE subject. For example, such a treatment can be administered to the SLE subject to prevent or slow the onset of the SLE disease activity event.

Clinical Measurement of SLE Disease Activity

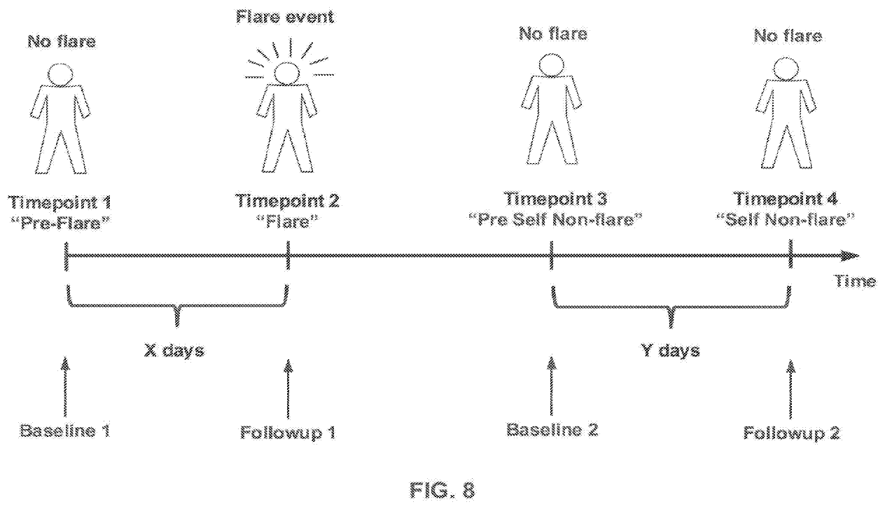

SLE clinical disease activity can be measured by any means known in the art. In some embodiments, the measurement of SLE clinical disease activity is the Safety of Estrogens in Lupus National Assessment—Systemic Lupus Erythematosus Disease Activity Index (SELENA-SLEDAI). The SELENA SLEDAI measurement may indicate that an SLE patient is not undergoing a clinical flare event, is undergoing a mild clinical flare event, is undergoing a moderate clinical flare event, or is undergoing a severe clinical flare event. Generally, diagnosis of a clinical flare event is conducted by a physician. Factors to be considered in diagnosing a clinical disease flare event using the SELENA-SLEDAI measurement include presence/absence of seizure(s), psychosis, organic brain syndrome, visual disturbance, cranial nerve disorder, lupus headache, cerebrovascular accidents, vasculitis, arthritis, myositis, urinary casts, hematuria, proteinuria, pyuria, rash, alocpecia, mucosal ulcers, pleurisy, pericarditis, low complement, increased DNA binding, fever, thrombocytopenia, and leukopenia. In some embodiments, SLE clinical disease activity is measured at the time of a follow-up visit by the same SLE patients in the cohort during which the SLE patient is undergoing a flare. Further details on the timing of samples obtained from SLE patients and/or SLE subject as well as the generation of a linear regression is described in further detail below in reference to FIG. 8.

Timing of Samples

Samples for Linear Regression

In various embodiments, the linear regression from which the predictive model obtains coefficients is generated from data obtained from a cohort of SLE patients that have been previously monitored over time (e.g., through clinic visits in which samples are drawn from the patients). In some embodiments, the cohort of SLE patients may include SLE patients that are not monitored over multiple clinic visits and instead, are monitored at individual clinic visits (e.g., one visit).

In one embodiment, the data obtained from the cohort of SLE patients includes expression levels of soluble mediators in samples obtained from the SLE patients at a time point (e.g., clinic visit). In one embodiment, the data include clinical measurements of SLE clinical disease activity at of the SLE patients at a time point (e.g., clinic visit) while the SLE patients are undergoing a flare event. In some embodiments, the data include both expression levels of soluble mediators in samples obtained from SLE patients at a first time point and clinical measurements of SLE clinical disease activity at of the SLE patients at a second time point. Therefore, the predictive model uses coefficients from a linear regression that is dependent on the relationship between the expression levels of soluble mediators in samples obtained from the SLE patients at the first time point and the clinical measurements of SLE clinical disease activity of the SLE patients at a second time point. In some embodiments, the coefficients are Spearman coefficients to enable non-parametric analyses of non-normally distributed soluble mediator expression level data.

Generally, the samples obtained from the SLE patients can be categorized as one of a pre-flare sample, a flare sample, a pre SNF sample, or a self non-flare sample. In various embodiments, each of a pre-flare sample, a flare sample, a pre SNF sample, or a self non-flare sample can be obtained from an individual in a cohort of SLE patients across multiple timepoints. To provide an example, reference is made to FIG. 8, which depicts an example timeline for monitoring an individual in the cohort of SLE patients. Generally, samples are obtained from an individual at each timepoint (e.g., timepoint 1, 2, 3, and 4). In various embodiments, each timepoint refers to a time during which the individual is visiting the clinic. Therefore, in some embodiments a sample is obtained from the individual during each clinic visit.

Pairs of samples are generally considered together in order to determine whether each of the samples in the pair are to be categorized as a pre-flare sample, a flare sample, a pre SNF sample, or a self non-flare sample. Specifically, as shown in FIG. 8, a sample may be obtained from the individual at timepoint 1 (also referred to as a baseline e.g., baseline 1). A number of days later (indicated as “X days” in FIG. 8), a second sample may be obtained from the individual at timepoint 2 (also referred to as a follow-up e.g., follow-up 1). At timepoint 1, the individual may be identified as not undergoing a flare (e.g., by a physician). At timepoint 2, however, the individual may be identified as undergoing a flare (e.g., by a physician). Therefore, the sample obtained from the individual at timepoint 1 can be categorized as a “pre-flare sample” (because the individual subsequently experienced a flare event at timepoint 2). Additionally, the sample obtained from the individual at timepoint 2 can be categorized as a “flare sample” (because the individual is undergoing a flare event at timepoint 2). The time period between timepoint 1 and timepoint 2, as described in this example as X days, refers to a period of imminent clinical disease flare (because the patient experienced a clinical flare event at timepoint 2).

Additionally, a sample can be obtained from the same individual at a timepoint 3 (also referred to as a baseline e.g., baseline 2). A number of days later (indicated as “Y days” in FIG. 8), another sample may be obtained from the individual at timepoint 4 (also referred to as a follow-up e.g., follow-up 2). At both timepoint 3 and timepoint 4, the individual may be identified as not undergoing a flare. For example, the individual may be clinically diagnosed by a physician as not undergoing a flare. Therefore, the sample obtained from the individual at timepoint 3 can be categorized as a “pre self non-flare sample” (because the individual subsequently did not experience a flare event at timepoint 4). Additionally, the sample obtained from the individual at timepoint 4 can be categorized as a “self non-flare sample.” The time period between timepoint 3 and timepoint 4, as described in this example as X days, refers to a non-flare period (because the patient did not experience a clinical flare event at timepoint 4).

It is to be understood that FIG. 8 depicts one particular example of how samples obtained from an individual in the SLE cohort can be categorized. Depending on how the individual presents at each timepoint, the samples can be differently categorized. For example, the individual may not be experiencing a flare at timepoint 1, timepoint 2, and timepoint 3, but is experiencing a clinical flare event at timepoint 4. Therefore, the sample obtained at timepoint 1 can be categorized as a pre self non-flare sample, the sample obtained at timepoint 2 is categorized as a self non-flare sample, the sample obtained at timepoint 3 is categorized as a pre-flare sample, and the sample obtained at timepoint 4 is categorized as a flare sample. In some embodiments, an individual in the SLE cohort is only monitored across a limited number of clinic visits (e.g., two clinic visits) and therefore, such an individual only provides a sample at each of the limited number of clinic visits. Depending on the status of the individual at those two visits, the two obtained samples can be categorized as pre-flare/flare samples or pre self non-flare/self non-flare samples. Such an individual may not be monitored over subsequent visits.

In various embodiments, the number of days between a baseline visit and a follow-up visit (denoted as “X days” or “Y days” in FIG. 8) is about 30 days (e.g., 1 month). In some embodiments, the number of days between a baseline visit and a follow-up visit is about 60 days (2 months), 90 days (3 months), 120 days, (4 months), 150 days (5 months), or 180 days (6 months). In some embodiments, the time between the baseline visit and the follow-up ranges from 30 days to 150 days. In some embodiments, the time between the baseline visit and the follow-up visit is between 50 and 120 days. In some embodiments, the time between the baseline visit and the follow-up visit is between 75 and 100 days.

Samples Obtained from a Subject for Predicting a Future SLE Disease Activity Event

In some embodiments, the SLE subject is monitored over time across multiple timepoints (e.g., clinic visits) as part of a cohort of SLE patients such that the data obtained from the SLE subject over the multiple clinic visits can be used a part of a dataset for generating the linear regression. Therefore, in predicting whether the SLE subject is likely to experience a future SLE disease activity event, the predictive model uses past data obtained from the SLE subject at one or more timepoints. Including the SLE subject as part of the cohort is beneficial in order to control for subject specific heterogeneity arising from the SLE disease.

In some embodiments, the SLE subject is not included in the cohort of SLE patients. Therefore, whether the SLE subject is likely to experience a SLE disease activity event can be predicted de novo (e.g., without prior data having been obtained from the SLE subject). In this scenario, the predictive model is able to generate a prediction (e.g., LFPI) for the SLE subject based on the linear regression that is generated from data obtained from a reference set of SLE patients (not including the SLE subject). This may be beneficial because the SLE subject need not be monitored over several timepoints (e.g., clinic visits) in order to predict whether the SLE subject is likely to experience a SLE disease activity event.

Treatment Methods

Having predicted a likelihood of a future SLE disease activity event in a SLE subject, a treatment may be administered to the SLE subject. Methods of treating a SLE subject may involve using standard therapeutic approaches. In general, the treatment of SLE involves treating elevated disease activity and trying to minimize the organ damage that can be associated with this increased inflammation and increased immune complex formation/deposition/complement activation. In various embodiments, treatment can include a therapeutic. Such a therapeutic can include corticosteroids or anti-malarial drugs. Certain types of lupus nephritis such as diffuse proliferative glomerulonephritis require bouts of cytotoxic drugs. These drugs include, most commonly, cyclophosphamide and mycophenolate. Hydroxychloroquine (HCQ) was approved by the FDA for lupus in 1955. Some drugs approved for other diseases are used for SLE ‘off-label.’ In November 2010, an FDA advisory panel recommended approving belimumab (BENLYSTA®) as a treatment for elevated disease activity seen in autoantibody-positive lupus patients. The drug was approved by the FDA in March 2011.

Due to the variety of symptoms and organ system involvement with SLE, its severity in an individual can be assessed and then treated. Mild or remittent disease may, sometimes, be safely left minimally treated with hydroxychloroquine alone. Nonsteroidal anti-inflammatory drugs and low dose steroids may also be used. Hydroxychloroquine (HCQ) is an FDA-approved antimalarial used for constitutional, cutaneous, and articular manifestations. Hydroxychloroquine has relatively few side effects, and there is evidence that it improves survival among people who have SLE and stopping HCQ in stable SLE patients led to increased disease flares in Canadian lupus patients. Disease-modifying antirheumatic drugs (DMARDs) are often times used off-label in SLE to decrease disease activity and lower the need for steroid use. DMARDs commonly in use are methotrexate and azathioprine. In more severe cases, medications that aggressively suppress the immune system (primarily high-dose corticosteroids and major immunosuppressants) are used to control the disease and prevent damage. Cyclophosphamide is used for severe glomerulonephritis, as well as other life-threatening or organ-damaging complications, such as vasculitis and lupus cerebritis. Mycophenolic acid is also used for treatment of lupus nephritis, but it is not FDA-approved for this indication.

Numerous immunosuppressive drugs are being actively tested for SLE. Rather than suppressing the immune system nonspecifically, as corticosteroids do, they target the responses of individual types of immune cells. Belimumab, or a humanized monoclonal antibody against B-lymphocyte stimulating factor (BlyS or BAFF), is FDA approved for lupus treatment and decreased SLE disease activity, especially in patients with baseline elevated disease activity and the presence of autoantibodies. Addition drugs, such as abatacept, epratuzinab, etanercept and others, are actively being studied in SLE patients and some of these drugs are already FDA-approved for treatment of rheumatoid arthritis or other disorders. Since a large percentage of people with SLE suffer from varying amounts of chronic pain, stronger prescription analgesics (pain killers) may be used if over-the-counter drugs (mainly nonsteroidal anti-inflammatory drugs) do not provide effective relief. Potent NSAIDs such as indomethacin and diclofenac are relatively contraindicated for patients with SLE because they increase the risk of kidney failure and heart failure.

Moderate pain is typically treated with mild prescription opiates such as dextropropoxyphene and co-codamol. Moderate to severe chronic pain is treated with stronger opioids, such as hydrocodone or longer-acting continuous-release opioids, such as oxycodone, MS Contin, or methadone. The fentanyl duragesic transdermal patch is also a widely used treatment option for the chronic pain caused by complications because of its long-acting timed release and ease of use. When opioids are used for prolonged periods, drug tolerance, chemical dependency, and addiction may occur. Opiate addiction is not typically a concern, since the condition is not likely to ever completely disappear. Thus, lifelong treatment with opioids is fairly common for chronic pain symptoms, accompanied by periodic titration that is typical of any long-term opioid regimen.

Intravenous immunoglobulins may be used to control SLE with organ involvement, or vasculitis. These therapeutics may reduce antibody production or promote the clearance of immune complexes from the body, even though their mechanism of action is not well-understood. Unlike immunosuppressives and corticosteroids, IVIGs do not suppress the immune system, so there is less risk of serious infections with these drugs.

Avoiding sunlight is the primary change to the lifestyle of SLE sufferers, as sunlight is known to exacerbate the disease, as is the debilitating effect of intense fatigue. These two problems can lead to patients becoming housebound for long periods of time. Drugs unrelated to SLE should be prescribed only when known not to exacerbate the disease. Occupational exposure to silica, pesticides and mercury can also make the disease worsen.

Renal transplants are the treatment of choice for end-stage renal disease, which is one of the complications of lupus nephritis, but the recurrence of the full disease in the transplanted kidney is common in up to 30% of patients.

Antiphospholipid syndrome is also related to the onset of neural lupus symptoms in the brain. In this form of the disease the cause is very different from lupus: thromboses (blood clots or “sticky blood”) form in blood vessels, which prove to be fatal if they move within the blood stream. If the thromboses migrate to the brain, they can potentially cause a stroke by blocking the blood supply to the brain. If this disorder is suspected in patients, brain scans are usually required for early detection. These scans can show localized areas of the brain where blood supply has not been adequate. The treatment plan for these patients often involves anticoagulation. Low-dose aspirin is prescribed for this purpose, although for cases involving thrombosis anticoagulants such as warfarin are used.

In various embodiments, administration of a therapeutic to the SLE subject can be via any common route so long as the target tissue is available via that route. Such routes include oral, nasal, buccal, rectal, vaginal or topical route. Alternatively, administration may be by orthotopic, intradermal, subcutaneous, intramuscular, intraperitoneal, or intravenous injection. The therapeutic may also be administered parenterally or intraperitoneally.

The therapeutic can be administered in a pharmaceutically or pharmacologically acceptable composition. The phrases “pharmaceutically or pharmacologically acceptable” refer to molecular entities and compositions that do not produce adverse, allergic, or other untoward reactions when administered to an animal or a human.

Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for implementing a predictive model, such as one described above, for the purposes of generating a LFPI predictive of a future SLE disease activity event.

Computer System

The methods of the invention, including the methods of analyzing soluble mediator levels for predicting a SLE disease activity event, are, in some embodiments, performed on a computer.

For example, the building and execution of a predictive model for generating a score (e.g., LFPI subscore or LFPI) can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a predictive model of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

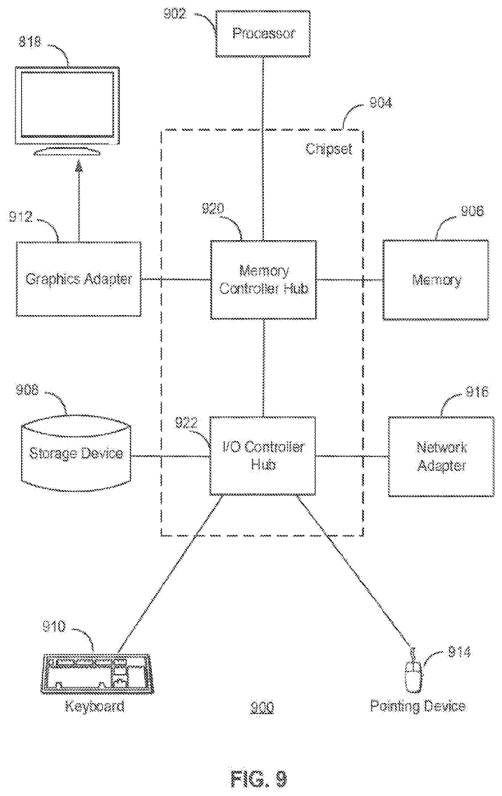

FIG. 9 illustrates an example computer 900 for implementing the predictive models shown in FIGS. 7A and 7B. The computer 900 includes at least one processor 902 coupled to a chipset 904. The chipset 904 includes a memory controller hub 920 and an input/output (I/O) controller hub 922. A memory 906 and a graphics adapter 912 are coupled to the memory controller hub 920, and a display 918 is coupled to the graphics adapter 912. A storage device 908, a pointing device 914, and network adapter 916 are coupled to the I/O controller hub 922. Other embodiments of the computer 900 have different architectures.

The storage device 908 is a non-transitory computer-readable storage medium such as a hard drive, compact disk read-only memory (CD-ROM), DVD, or a solid-state memory device. The memory 906 holds instructions and data used by the processor 902. The input interface 914 is a touch-screen interface, a mouse, track ball, or other type of pointing device, a keyboard, or some combination thereof, and is used to input data into the computer 900. In some embodiments, the computer 900 may be configured to receive input (e.g., commands) from the input interface 914 via gestures from the user. The graphics adapter 912 displays images and other information on the display 918. The network adapter 916 couples the computer 900 to one or more computer networks.

The computer 900 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 908, loaded into the memory 906, and executed by the processor 902.

The types of computers 900 can vary from the embodiments described herein. For example, the computer 900 can lack some of the components described above, such as graphics adapters 912, pointing device 914, and displays 918. In some embodiments, a computer 900 can include a processor 902 for executing instructions stored on a memory 906.

Kits

Also disclosed herein are kits for analyzing soluble mediator levels for predicting a SLE disease activity event. Such kits can include reagents for detecting expression levels of one or markers and instructions for predicting a SLE disease activity event based on the detected expression levels of soluble mediators.

A kit can comprise a set of reagents for generating a dataset via at least one assay. The set of reagents enable the detection of quantitative expression levels of one or more Th1 cytokines, chemokines or adhesion molecules, TNRF superfamily member molecules, regulatory mediator molecules, and SLE mediator molecules. The set of reagents may further enable the detection of quantitative expression levels of one or more innate cytokines, Th2 cytokines, and Th17 cytokines. In certain aspects, the reagents include one or more antibodies that bind to one or more of the markers. The antibodies may be monoclonal antibodies or polyclonal antibodies. In some aspects, the reagents can include reagents for performing ELISA including buffers and detection agents.

In some embodiments, such kits can comprise a carrier, package or container that is compartmentalized to receive one or more containers such as vials, tubes, and the like, each of the container(s) comprising one of the separate elements to be used in the method. The kit of the invention can comprise the container described above and one or more other containers comprising materials desirable from a commercial end user standpoint, including buffers, diluents, filters, and package inserts with instructions for use. In addition, a label can be provided on the container to indicate that the composition is used for a specific therapeutic application, and can also indicate directions for either in vivo or in vitro use, such as those described above. Directions and or other information can also be included on an insert which is included with the kit. In some embodiments, kits contemplate the assemblage of agents for assessing levels of the biomarkers discussed above along with one or more of an SLE therapeutic and/or a reagent for assessing antinuclear antibody (ANA) testing and/or anti-extractable nuclear antigen (anti-ENA), as well as controls for assessing the same.

A kit can include instructions for use of a set of reagents. For example, a kit can include instructions for performing at least one assay such as an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassay selected from RIA, immunofluorescence, immunochemiluminescence, immunoelectrochemiluminescence, immunoelectrophoretic, a competitive immunoassay, and immunoprecipitation.

In addition to the above components, the subject kits will further include instructions for practicing the subject methods. These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

Systems

Also disclosed herein are systems for analyzing soluble mediator levels for predicting a SLE disease activity event. Such a system can include a set of reagents for detecting expression levels of one or soluble mediators, an apparatus configured to receive a mixture of one or more reagents and a test sample obtained from a subject to measure the expression levels of the soluble mediators, and a computer system communicatively coupled to the apparatus to obtain the measured expression levels and to determine a score predictive of the likelihood of a SLE disease activity event in the SLE patient.

The set of reagents enable the detection of quantitative expression levels of one or more Th1 cytokines, chemokines or adhesion molecules, TNRF superfamily member molecules, regulatory mediator molecules, and SLE mediator molecules. The set of reagents may further enable the detection of quantitative expression levels of one or more innate cytokines, Th2 cytokines, and Th17 cytokines. In certain aspects, the reagents include one or more antibodies that bind to one or more of the markers. The antibodies may be monoclonal antibodies or polyclonal antibodies. In some aspects, the reagents can include reagents for performing ELISA including buffers and detection agents.

The apparatus is configured to detect expression levels of soluble mediators in a mixture of a reagent and test sample. For example, the apparatus can determine quantitative expression levels of soluble mediators through an immunologic assay or assay for nucleic acid detection. The mixture of the reagent and test sample may be presented to the apparatus through various containers, examples of which include wells of a well plate (e.g., 96 well plate), a vial, or tube. As such, the apparatus may have an opening (e.g., a slot, a cavity, an opening, a sliding tray) that can receive the container including the reagent test sample mixture and perform a reading to generate quantitative expression values of soluble mediators. Examples of an apparatus include a plate reader (e.g., a luminescent plate reader, absorbance plate reader, fluorescence plate reader), a spectrometer, and a spectrophotometer.

The computer system communicates with the apparatus to receive the quantitative expression values of soluble mediators. The computer system analyzes the quantitative expression values by applying a predictive model and determines a likelihood of a SLE disease activity event in the subject. Example computer systems are described herein.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Multivariable Biomarker Panel for Predicting Likelihood of Impending SLE Flare

Methods: Patient Selection

Experiments were performed in accordance with the Helsinki Declaration and approved by the Institutional Review Board of the Oklahoma Medical Research Foundation. Study participants (Table 1) were enrolled in their respective cohorts after written informed consent; the SLE patients in the Oklahoma Cohort for Rheumatic Diseases (OCRD) and matched, healthy controls from the Oklahoma Immune Cohort (OIC). Demographic and clinical information were collected as previously described (45), including medication usage, clinical laboratory values, disease activity, and SELENA-SLEDAI defined flare (Table 1); Severe flares were uncommon and not assessed independently (45). Female European American (EA)(n=49) and African American (AA) (n=41) SLE patients (meeting≥4 ACR classification criteria (45)) with plasma samples available during a pre-flare clinic visit were compared to samples drawn from the same individuals in a comparable time period of clinic visits with no associated SELENA-SLEDAI flare. Generally, a pre-flare timepoint was an average of 100.4±40.1 days prior to flare whereas a pre-self non-flare (pre-SNF) was an average of 95.8±40.0 days prior to a self non-flare time point. See Table 1 for further details on the patient population. SLE patients were matched by age (5 years) and race to healthy control individuals. Undiluted plasma has been serially collected from SLE patients (OCRD) and healthy individuals (OIC) and stored at −80° in the Oklahoma Rheumatic Diseases Research Core Center (ORDRCC), CAP-certified, biorepository at OMRF.

Methods: Soluble Mediator Determination

Plasma levels of BLyS (R&D Systems/Bio-techne, Minneapolis, Minn.) and APRIL (eBioscience/ThermoFisher Scientific, Waltham, Mass.) were determined by enzyme-linked immunosorbent assay (ELISA), per the manufacturer protocol. An additional fifty analytes, including innate and adaptive cytokines, chemokines, and soluble TNFR superfamily members (Table 2A and 2B), were assessed by xMAP multiplex assays (R&D Systems/Bio-techne) (46).

Data were analyzed on the Bio-Rad BioPlex 200® array system (Bio-Rad Technologies, Hercules, Calif.), with a lower boundary of 100 beads per sample/analyte. Median fluorescence intensity for each analyte was interpolated from 5-parameter logistic nonlinear regression standard curves. Analytes below the detection limit were assigned a value of 0.001 pg/mL. A known control serum was included on each plate (Cellgro human AB serum, Cat #2931949, L/N #M1016). Mean inter-assay coefficient of variance (CV) of multiplexed bead-based assays for cytokine detection has previously been shown to be 10-14% (47, 48), and a similar average CV (10.5%) across the analytes in this assay was obtained using healthy control serum. Intra-assay precision of duplicate wells averaged <10% CV in each 25-plex assay.

Methods: Statistical Analysis

Plasma mediator concentration. Concentrations of plasma mediators were compared between pre-flare SLE patients and self non-flare samples by Wilcoxon matched-pairs test and adjusted for multiple comparisons using the False Discovery Rate (FDR) via the Benjamini-Hochberg procedure (using R version 2.15.3). Differences between pre-flare and self non-flare samples, and matched healthy controls were determined by Kruskal-Wallis test with correction by Dunn's multiple comparison. Differences in the LFPI or soluble mediators between SLE patients with vs. without various clinical manifestations were compared by Mann-Whitney test. Except where noted, analyses were performed using GraphPad Prism 6.02 (GraphPad Software, San Diego, Calif.).

Lupus Flare Prediction Index (LFPI). To compare the overall level of inflammation in pre-flare vs. non-flare SLE patients (at baseline) in relationship to disease activity at flare (post-vaccination), a LFPI was derived by the cumulative contribution of all pre-flare plasma mediators assessed in relationship to SELENA-SLEDAI disease activity at flare (49, 50). Briefly, the concentration of all plasma analytes were log-transformed and standardized; (observed analyte value)−(mean analyte value across all SLE patients assessed [Flare, Non-flare, or Self non-flare])/(standard deviation of all SLE patients assessed [Flare, Non-flare, or Self non-flare]. Self non-flare patients refer to SLE patients that have been tracked over sufficient time such that there are previously obtained soluble mediator expression levels for the self non-flare patients corresponding to a disease flare period (e.g., a pre-flare sample and a subsequent flare sample) as well as a non-flare period (e.g., a pre self non-flare sample and a subsequent self non-flare sample). Non-flare SLE patients refer to unique SLE patients that are not tracked over multiple timepoints. For the timepoint in which the non-flare SLE patient provides a sample, the non-flare SLE patients is not experiencing a clinical disease flare.

Spearman coefficients of each analyte were generated from a linear regression model testing associations between the flare SELENA-SLEDAI disease activity scores and each pre-flare soluble mediator. The transformed and standardized soluble mediator levels were weighted by the respective Spearman coefficients and summed for a total, global LFPI score (49, 50). By generating the weights, the inflammatory mediators that were most differentially altered at baseline between pre-flare and non-flare SLE patients in their associations with SELENA-SLEDAI scores at time of disease flare contributed most to the score and therefore the overall level of inflammation correlating with disease flare, Table 2A and 2B.

Multivariable, machine learning analysis. To determine which mediators best differentiated pre-flare from self non-flare (SNF) samples, a random forest (RF) classification algorithm (51) was implemented using the randomForest R packages (version 4.6-7). Default settings were used (mtry=\sqrt{number of variables}, importance=TRUE, and proximity=TRUE) except that ntree was set to 2,000. For each forest, a randomly selected training set (⅔ of total samples) was used to generate an ensemble of decision trees. The performance of each RF was evaluated using accuracy (1−out of bag (OOB) error). Variables were selected using the stepwise-like algorithm of Genuer and Tuleau-Malot (51) to predict imminent clinical disease flare (pre-flare). Final RF models identified the set of predictors that independently contributed to the differentiation of future SLE patients. These findings were confirmed using the same approach applied to gradient tree boosting, extreme gradient boosting (XGBoost), using the xgboost R package (52).

Results: A Weighted Luaus Flare Prediction Index (LFPI) Correlates with Impending Flare

To determine the correlation and relative contribution of pre-flare inflammatory and regulatory soluble analytes to SLE disease flare risk, LFPI subscores were combined to serve as a Lupus Flare Prediction Index (LFPI) that has been previously shown to identify SLE patients with imminent clinical disease flare (49, 50). A distinct advantage of the following approach is that it does not require cut-offs for each cytokine/chemokine to establish positivity, and gives impact to those untransformed pre-flare analytes with stronger correlations (Spearman correlation coefficients) to disease activity at time of flare (Table 2A, right panel).

Briefly, 1. The concentration of each baseline plasma mediators was log-transformed for each SLE patient; 2. Each log-transformed soluble mediator level for each SLE patient was standardized: (observed value)−(mean value of all SLE patients assessed [Pre-flare and Pre-SNF])/(standard deviation of all SLE patients assessed [Pre-flare and Pre-SNF]); 3. Spearman coefficients were generated from a linear regression model testing associations between the SELENA-SLEDAI disease activity score at follow-up in each SLE patient and each soluble mediator at baseline (Spearman r, Table 2A, right panel); 4. The transformed and standardized soluble mediator levels at baseline were weighted (multiplied) by their respective Spearman coefficients (Spearman r, Table 2A, right panel). Soluble mediators that best distinguished Pre-flare from Pre-SNF patients most significantly contributed to the SMS (Table 2B). 5. For each patient, the log transformed, standardized and weighted values for each of the soluble mediators to be included in the LFPI, were summed to calculate a total LFPI. For example, the values shown in the “Pre-flare median” column in Table 3B was summated to determine a pre-flare LFPI whereas the values shown in the “Pre-SNF median” column in Table 3B was summated to determine a pre-SNF LFPI.

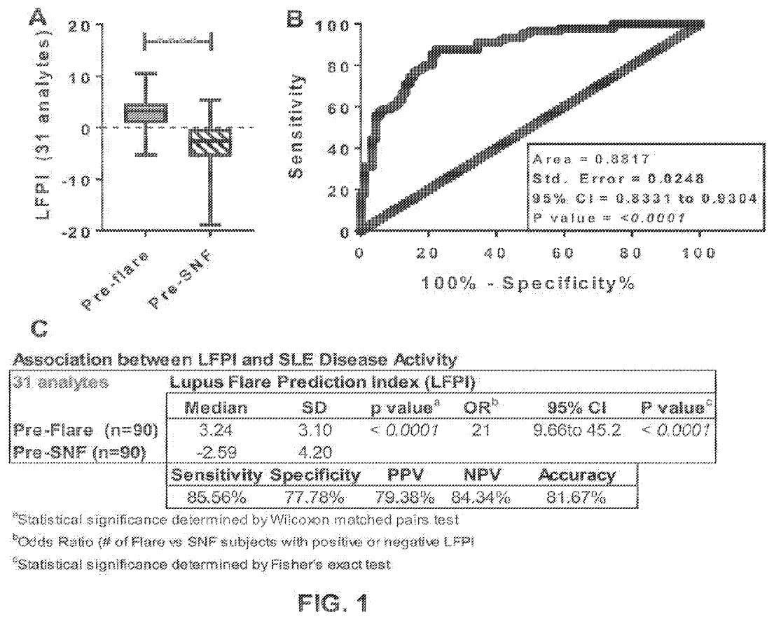

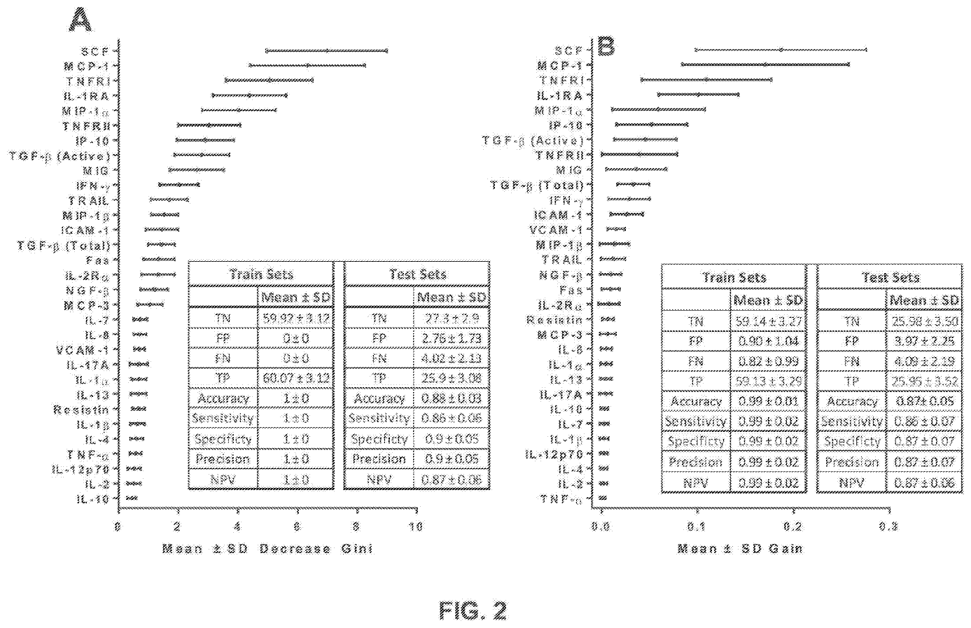

Based on the univariate analyses (Table 2A and 2B) and the performance of the LFPI, 20 plasma mediators were eliminated from the LFPI and, leaving a 31 mediator informed LFPI that highly significantly differentiated Pre-flare from Pre-SNF samples (FIG. 1; list of 31 mediators outlined in FIG. 2). The LFPI was significantly higher in the same SLE patients with impending flare versus comparable non-flare periods (median soluble analyte score 3.24 [Pre-flare] vs. −2.59 [Pre-SNF], p<0.0001; FIG. 1A,C). The AUC for this 31 mediator-informed LFPI was 0.8817±0.0248 (p<0.0001, FIG. 1B), with 85.6% Sensitivity, 77.8% Specificity, and 81.7% Accuracy (FIG. 1C). Compared to non-flare periods in the same patients, pre-flare samples were 21 times more likely to have a positive LFPI score (FIG. 1C). Seventy-seven of 90 Pre-flare samples had positive LFPI scores, all of which decreased during a comparable periods of non-flare, while 70/90 non-flare SLE patients had negative LFPI scores.

Results: Machine Learning Identifies Mediators that Best Differentiate SLE Patients with Impending Clinical Disease Flare.

Further refinement of biomarker/mediators was pursued to improve both performance and cost efficiency for future clinical applications. Due to the number of highly significant mediators that differentiated Pre-flare from Pre-SNF samples in the univariate analyses (Table 2A/2B), random forest (51) was employed to identify a reduced set of mediators (FIG. 2A). A comparable gradient tree boosting method, XGBoost (52), was performed which revealed similar variable importance rankings of the plasma mediators (FIG. 2B). Both random forest (FIG. 2A) and XGBoost (FIG. 2B) similarly identified the top nine informative soluble immune mediators, including SCF, MCP-1/CCL2, TNFRI. IL-1RA, MIP-1α/CCL3, TNFRII, IP-10/CXLC10, Active TGF-β1, and MIG/CXCL9. Other significant mediators, including IFN-γ. Total TGF-β1, Fas, IL-2Rα, ICAM-1, MIP-1β/CCL4, and TRAIL were among the next highest ranked block of mediators by both random forest (FIG. 2A) and XGBoost (FIG. 2B). Mediators that less consistently differentiated Pre-flare vs. Pre-SNF samples by random forest and XGBoost included IL-10, IL-2, IL-12p70, TNF-α, IL-4, and IL-1β (FIG. 2), despite their highly significant status in univariate analyses (Table 2A/2B).

Results: Variable Importance Applied to the LFPI Improves Performance

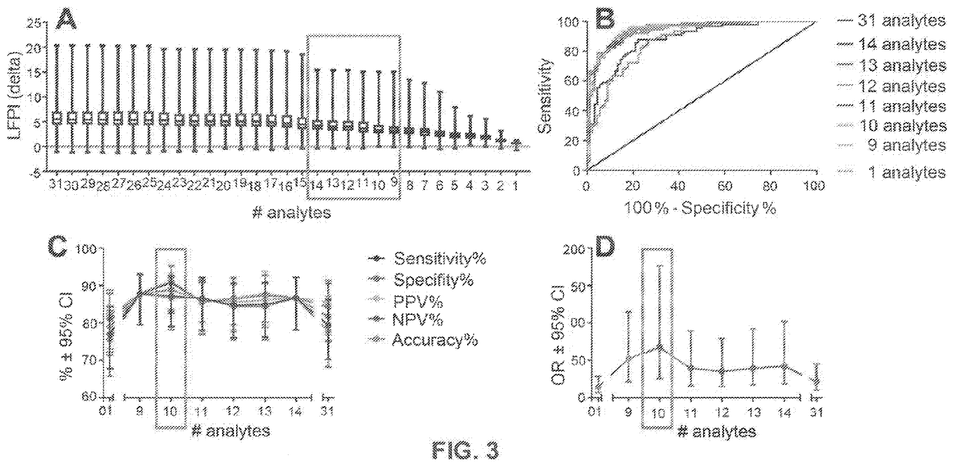

Based on the random forest Variable Importance rankings (FIG. 2), the optimal range of soluble mediators that best inform the LFPI was determined by utilizing the forward and backward stepwise progression method of Genuer and Tuleau-Malot (51) (FIG. 3). Briefly, the forward stepwise progression method is as follows: Start with 1 mediator (most important as determined by random forest Variable Importance) and add subsequent (ranked) mediators until optimal LFPI is achieved. Briefly, the backward stepwise progression method is as follows: start with all biomarkers and remove the lowest ranked mediator in a stepwise fashion until optimal LFPI is achieved. Forward progression (add soluble mediators until reach optimal differentiation of Pre-flare and Pre-SNF samples) led to an optimum of nine soluble mediators, while backward progression (subtraction of soluble mediators) revealed an optimum of fourteen soluble mediators, reflected as a “middle” change in the LFPI between Pre-flare and Pre-SNF samples (FIG. 3A). The optimal combination of soluble mediators was determined based on the combination of mediators that resulted in the highest predictive model performance (e.g., highest sensitivity, specificity, NPV, PPV, accuracy, and odds ratio of the LFPI score) in its ability to predict the highest risk of imminent clinical disease flare.

All fourteen top mediators both significantly differentiated Pre-flare and Pre-SNF samples by plasma concentration levels (Table 3A, left panel) and highly significantly correlated with disease activity at time of concurrent flare/non-flare at a subsequent, follow-up clinic visit (Table 3A, right panel). Ten of the fourteen mediators also significantly contributed to the LFPI as single analytes (Table 3B).

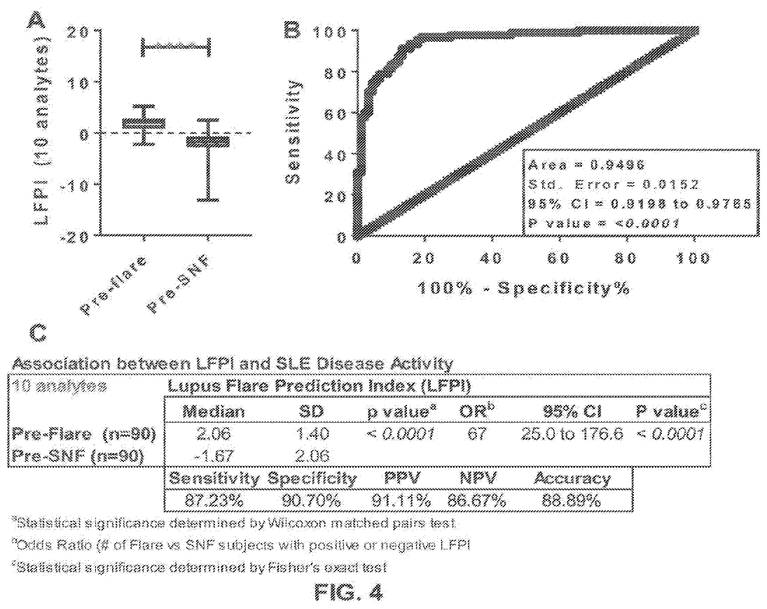

The AUC when including 9-14 analytes in the LFPI was similar and increased compared to including all 31 analytes in the LFPI or the top analyte alone (SCF), FIG. 3B. Looking more closely at sensitivity, specificity, PPV, NPV, and accuracy, including ten analytes in the LFPI led to the best performance (FIG. 3C), as well as the best odds ratio (OR) of a Pre-flare sample having a positive LFPI (FIG. 3D). When using the top 10 analytes (SCF, MCP-1/CCL2, TNFRI, IL-1RA, MIP-1α/CCL3, TNFRII, IP-10/CXLC10, Active TGF-β1, MIG/CXCL9, MIG/CXCL9, and IFN-γ) to inform the LFPI (FIG. 2A and Table 3B), the LFPI continued to highly significantly differentiate Pre-flare vs. Pre-SNF samples (FIG. 4A). The AUC improved to 0.9496±0.0152 (p<0.0001, FIG. 4B), and the sensitivity (87.2%), specificity (90.7%), and accuracy (88.9%) of the LFPI also improved (FIG. 4C), with Pre-flare samples being 67 times more likely to have a positive LFPI (FIG. 4C).

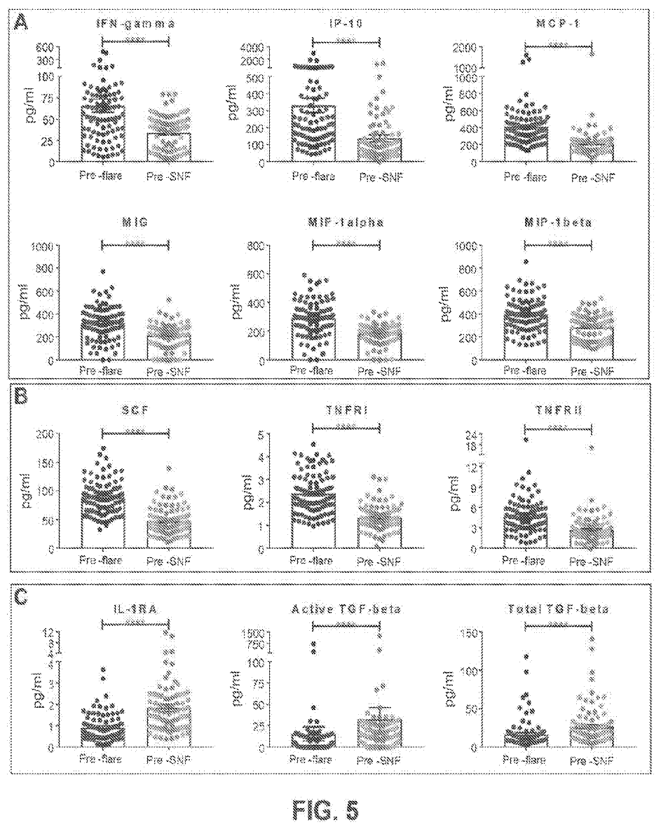

A number of Pre-flare samples within this SLE cohort contain relatively high levels of IFN-associated mediators, including IFN-γ, IP-10/CXCL10, MCP-1/CCL2, MIG/CXCL9, MIP-1α/CCL3, and MIP-1β/CCL4 (FIG. 5A). In addition, there are a number of samples with highly elevated levels of the pro-inflammatory mediator, SCF, as well as TNFRI and TNFRII (FIG. 5B). Conversely, increased levels of the regulatory mediators IL-1RA. Active TGF-1, and Total TGF-β1 in Pre-SNF samples are observed (FIG. 5C). While the overall level of these mediators is highly significantly different between Pre-flare and Pre-SNF samples (p<0.0001, FIG. 5), there is heterogeneity among the samples, such that some samples exhibit higher levels of some significant mediators than others. Example 2: Multivariate Biomarker Panel for Predicting Likelihood of Organ Damage

Refined LFPI and Top Mediators at Baseline Differentiate Organ System Inflammation at Follow-Up

In addition to being able to differentiate impending disease flare from non-flare, the LFPI, and top mediators which inform it, was tested for differentiating those SLE patients with organ system manifestations at a future clinic visit. Specifically, as demonstrated in Table 1 and the results below, the LFPI calculated for SLE patients based on their soluble mediator expression levels at a baseline visit (e.g., a first clinic visit) can be used to predict organ system manifestations at a subsequent timepoint (e.g., ˜100 days in the future).

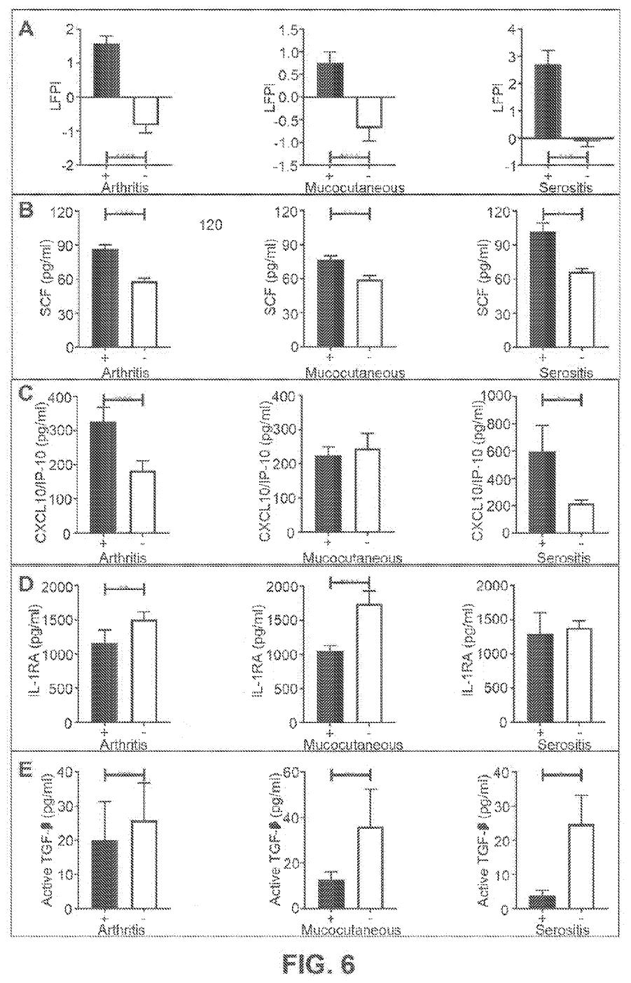

SELENA-SLEDAI defined organ system manifestations that were most significant between flare and non-flare at follow-up in this cohort of SLE patients include arthritis (n=59 [66%] flare vs. n=5 [6%] non-flare, p<0.0001), mucocutaneous (rash, alopecia, and/or mucosal ulcers, n=70 [78%] flare vs. n=25 [28%] non-flare, p<0.0001), and serositis (n=8 [9%] flare vs. 0 non-flare, p=0.0066), Table 1. Reference is made to FIGS. 6A-6E which depict baseline LFPI or expression levels of soluble mediators for patients that will experience an arthritis, mucocutaneous, or serositis organ system manifestation (filled in bars) as compared to LFPI or expression levels of soluble mediators for patients that will not experience an organ system manifestation (non-filled bars) at follow-up.

Baseline LFPI significantly differentiates SLE patients with these organ system manifestations at a future clinic visit (p<0.001, FIG. 6A). Baseline SCF, which was found to be the most significant differentiator of Pre-flare and Pre-SNF plasma samples by random forest an XG Boost (FIG. 2), was also significantly elevated in SLE patients with these organ system manifestations at follow-up (p<0.01, FIG. 6B). Yet, baseline IP-10/CXCL10 was only significantly elevated in those SLE patients with arthritis or serositis at their follow-up visit (p<0.01, FIG. 6C). Similar heterogeneity was found among the regulatory mediators as well, with baseline IL-1RA being elevated in patients without arthritis or mucocutaneous manifestations at follow-up (p<0.01, FIG. 6D), while Active TGF-b1 was most elevated in patients without arthritis, mucocutaneous, or serositis manifestations at follow-up (p<0.05, FIG. 6E). These findings once again support the utility of the LFPI in its ability to overcome clinical heterogeneity within SLE to identify flare patients and subsets of SLE patients with more debilitating organ system manifestations.

This study confirms inflammatory and regulatory pathways potentially dysregulated prior to the occurrence of a lupus flare before clinical symptoms are reported. Plasma samples and clinical data were evaluated from clinic visits of EA and AA SLE patients in the Oklahoma Cohort for Rheumatic Diseases (OCRD) and matched, healthy controls from the Oklahoma Immune Cohort (OIC). Using an xMAP multiplex approach, SLE patients with impending disease flare were found to have increased pre-flare inflammatory adaptive cytokines, chemokines, and shed TNFR superfamily members, with decreased regulatory mediators of inflammation, compared to the same patients with during a non-flare time period. These results enabled the refinement of a LFPI score that reflects pre-flare immune status in SLE patients who go on to flare.

### APPENDIX

Appendix A is a document 3 pages in length (including title slip sheet) describing embodiments related to methods for analyzing expression values of a panel of biomarkers using a predictive model for classifying subjects with high or low annualized multiple sclerosis relapse rates. Appendix A is hereby incorporated by reference, in its entirety, for all purposes.

It should be noted that the language used in Appendix A has been principally selected for readability and instructional purposes, and it may not have been selected to delineate or circumscribe the inventive subject matter. Accordingly, the disclosure of Appendix A is intended to be illustrative, but not limiting, of the scope of the invention.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

While various embodiments of the invention have been described herein, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

1. D'Cruz D P, Khamashta M A, Hughes G R. Systemic lupus erythematosus. Lancet. 2007; 369(9561):587-96.
2. Yen E Y, Singh R R. Brief Report: Lupus—An Unrecognized Leading Cause of Death in Young Females: A Population-Based Study Using Nationwide Death Certificates, 2000-2015. Arthritis & rheumatology. 2018; 70(8):1251-5.
3. Petri M. Disease activity assessment in SLE: do we have the right instruments? Ann Rheum Dis. 2007; 66 Suppi 3:iii61-4.
4. Ruperto N, Hanrahan L M, Alarcon G S, Belmont H M, Brey R L, Brunetta P, et al. International consensus for a definition of disease flare in lupus. Lupus. 2011; 20(5):453-62.
5. Petri M, Genovese M, Engle E, Hochberg M. Definition, incidence, and clinical description of flare in systemic lupus erythematosus. A prospective cohort study. Arthritis Rheum. 1991; 34(8):937-44.
6. Petri M, Singh S, Tesfasyone H, Malik A. Prevalence of flare and influence of demographic and serologic factors on flare risk in systemic lupus erythematosus: a prospective study. J Rheumatol. 2009; 36(11):2476-80.
7. van Vollenhoven R F, Petri M, Wallace D J, Roth D A, Molta C T, Hammer A E, et al. Cumulative Corticosteroid Dose Over Fifty-Two Weeks in Patients With Systemic Lupus Erythematosus: Pooled Analyses From the Phase IlIl Belimumab Trials. Arthritis Rheumatol. 2016; 68(9):2184-92.
8. Lau C S, Mak A. The socioeconomic burden of SLE. Nat Rev Rheumatol. 2009; 5(7):400-4.
9. Petri M, Purvey S, Fang H, Magder L S. Predictors of organ damage in systemic lupus erythematosus: the Hopkins Lupus Cohort. Arthritis Rheum. 2012; 64(12):4021-8.
10. Gonzalez L A, Toloza S M, McGwin G, Jr., Alarcon G S. Ethnicity in systemic lupus erythematosus (SLE): its influence on susceptibility and outcomes. Lupus. 2013; 22(12):1214-24.
11. Ugarte-Gil M F, Acevedo-Vasquez E, Alarcon G S, Pastor-Asurza C A, Alfaro-Lozano J L, Cucho-Venegas J M, et al. The number of flares patients experience impacts on damage accrual in systemic lupus erythematosus: data from a multiethnic Latin American cohort. Ann Rheum Dis. 2015; 74(6):1019-23.
12. Bruce I N, O'Keeffe A G, Farewell V, Hanly J G, Manzi S, Su L, et al. Factors associated with damage accrual in patients with systemic lupus erythematosus: results from the Systemic Lupus International Collaborating Clinics (SLICC) Inception Cohort. Ann Rheum Dis. 2015; 74(9):1706-13.
13. Alarcon G S, McGwin G, Jr., Petri M, Ramsey-Goldman R, Fessler B J, Vila L M, et al. Time to renal disease and end-stage renal disease in PROFILE: a multiethnic lupus cohort. PLoS Med. 2006; 3(10):e396.
14. Sacks J, Helmick C, Langmaid G, Sniezek J. From the Centers for Disease Control and Prevention. Trends in deaths from systemic lupus erythematosus—United States, 1979-1998. JAMA. 2002; 287(20):2649-50.
15. Jacobi A M, Odendahl M, Reiter K, Bruns A, Burmester G R, Radbruch A, et al. Correlation between circulating CD27high plasma cells and disease activity in patients with systemic lupus erythematosus. Arthritis Rheum. 2003; 48(5):1332-42.
16. Yang D H, Chang D M, Lai J H, Lin F H, Chen C H. Significantly higher percentage of circulating CD27(high) plasma cells in systemic lupus erythematosus patients with infection than with disease flare-up. Yonsei medical journal. 2010; 51(6):924-31.
17. Petri M A, van Vollenhoven R F, Buyon J, Levy R A, Navarra S V, Cervera R, et al. Baseline Predictors of Systemic Lupus Erythematosus Flares: Data From the Combined Placebo Groups in the Phase III Belimumab Trials. Arthritis Rheum. 2013; 65(8):2143-53.
18. Ho A, Magder L S, Barr S G, Petri M. Decreases in anti-double-stranded DNA levels are associated with concurrent flares in patients with systemic lupus erythematosus. Arthritis and rheumatism. 2001; 44(10):2342-9.
19. Kavanaugh A F, Solomon D H, American College of Rheumatology Ad Hoc Committee on Immunologic Testing G. Guidelines for immunologic laboratory testing in the rheumatic diseases: anti-DNA antibody tests. Arthritis and rheumatism. 2002; 47(5):546-55.
20. ter Borg E J, Horst G, Hummel E J, Limburg P C, Kallenberg C G. Measurement of increases in anti-double-stranded DNA antibody levels as a predictor of disease exacerbation in systemic lupus erythematosus. A long-term, prospective study. Arthritis and rheumatism. 1990; 33(5):634-43.
21. Froelich C J, Wallman J, Skosey J L, Teodorescu M. Clinical value of an integrated ELISA system for the detection of 6 autoantibodies (ssDNA, dsDNA, Sm, RNP/Sm, SSA, and SSB). The Journal of rheumatology. 1990; 17(2):192-200.
22. Weinstein A, Bordwell B, Stone B, Tibbetts C, Rothfield N F. Antibodies to native DNA and serum complement (C3) levels. Application to diagnosis and classification of systemic lupus erythematosus. The American journal of medicine. 1983; 74(2):206-16.
23. Buyon J, Furie R, Putterman C, Ramsey-Goldman R, Kalunian K, Barken D, et al. Reduction in erythrocyte-bound complement activation products and titres of anti-Clq antibodies associate with clinical improvement in systemic lupus erythematosus. Lupus science & medicine. 2016; 3(1):e000165.
24. Putterman C, Furie R, Ramsey-Goldman R, Askanase A, Buyon J, Kalunian K, et al. Cell-bound complement activation products in systemic lupus erythematosus: comparison with anti-double-stranded DNA and standard complement measurements. Lupus science & medicine. 2014; 1(1):e000056.
25. Davas E M, Tsirogianni A, Kappou I, Karamitsos D, Economidou I, Dantis P C. Serum IL-6, TNFalpha, p55 srTNFalpha, p75srTNFalpha, srlL-2alpha levels and disease activity in systemic lupus erythematosus. Clin Rheumatol. 1999; 18(1):17-22.
26. Chun H Y, Chung J W, Kim H A, Yun J M, Jeon J Y, Ye Y M, et al. Cytokine IL-6 and IL-10 as biomarkers in systemic lupus erythematosus. J Clin Immunol. 2007; 27(5):461-6.
27. Gomez D, Correa P A, Gomez L M, Cadena J, Molina J F, Anaya J M. Th1/Th2 cytokines in patients with systemic lupus erythematosus: is tumor necrosis factor alpha protective? Semin Arthritis Rheum. 2004; 33(6):404-13.
28. Tokano Y, Morimoto S, Kaneko H, Amano H, Nozawa K, Takasaki Y, et al. Levels of IL-12 in the sera of patients with systemic lupus erythematosus (SLE)-relation to Th1- and Th2-derived cytokines. Clin Exp Immunol. 1999; 116(1):169-73.
29. Shah K, Lee W W, Lee S H, Kim S H, Kang S W, Craft J, et al. Dysregulated balance of Th17 and Th1 cells in systemic lupus erythematosus. Arthritis Res Ther. 2010; 12(2):R53.
30. Mok M Y, Wu H J, Lo Y, Lau C S. The Relation of Interleukin 17 (IL-17) and IL-23 to Th1/Th2 Cytokines and Disease Activity in Systemic Lupus Erythematosus. J Rheumatol. 2010.
31. Chen D Y, Chen Y M, Wen M C, Hsieh T Y, Hung W T, Lan J L. The potential role of Th17 cells and Th17-related cytokines in the pathogenesis of lupus nephritis. Lupus. 2012; 21(13):1385-96.
32. Becker-Merok A, Eilertsen G O, Nossent J C. Levels of Transforming Growth Factor-{beta} Are Low in Systemic Lupus Erythematosus Patients with Active Disease. J Rheumatol. 2010.
33. Alexander T, Sattler A, Templin L, Kohler S, Gross C, Meisel A, et al. Foxp3+ Helios+ regulatory T cells are expanded in active systemic lupus erythematosus. Ann Rheum Dis. 2013; 72(9):1549-58.
34. Golding A, Hasni S, lllei G, Shevach E M. The Percentage of FoxP3+Helios+ Treg Cells Correlates Positively With Disease Activity in Systemic Lupus Erythematosus. Arthritis Rheum. 2013; 65(11):2898-906.
35. Bonelli M, von Dalwigk K, Savitskaya A, Smolen J S, Scheinecker C. Foxp3 expression in CD4+ T cells of patients with systemic lupus erythematosus: a comparative phenotypic analysis. Ann Rheum Dis. 2008; 67(5):664-71.
36. Miyara M, Amoura Z, Parizot C, Badoual C, Dorgham K, Trad S, et al. Global natural regulatory T cell depletion in active systemic lupus erythematosus. J Immunol. 2005; 175(12):8392-400.
37. Alvarado-Sanchez B, Hernandez-Castro B, Portales-Perez D, Baranda L, Layseca-Espinosa E, Abud-Mendoza C, et al. Regulatory T cells in patients with systemic lupus erythematosus. J Autoimmun. 2006; 27(2):110-8.
38. Vargas-Rojas M I, Crispin J C, Richaud-Patin Y, Alcocer-Varela J. Quantitative and qualitative normal regulatory T cells are not capable of inducing suppression in SLE patients due to T-cell resistance. Lupus. 2008; 17(4):289-94.
39. Ma J, Yu J, Tao X, Cai L, Wang J, Zheng S G. The imbalance between regulatory and IL-17-secreting CD4+ T cells in lupus patients. Clin Rheumatol. 2010; 29(11):1251-8.
40. Croft M, Benedict C A, Ware C F. Clinical targeting of the TNF and TNFR superfamilies. Nat Rev Drug Discov. 2013; 12(2):147-68.
41. Tinazzi E, Puccetti A, Gerdi R, Rigo A, Migliorini P, Simeoni S, et al. Serum DNase 1, soluble Fas/FasL levels and cell surface Fas expression in patients with SLE: a possible explanation for the lack of efficacy of hrDNase I treatment. Int Immunol. 2009; 21(3):237-43.
42. Desai-Mehta A, Lu L, Ramsey-Goldman R, Datta S K. Hyperexpression of CD40 ligand by B and T cells in human lupus and its role in pathogenic autoantibody production. J Clin Invest. 1996; 97(9):2063-73.
43. Dillon S R, Harder B, Lewis K B, Moore M D, Liu H, Bukowski T R, et al. B-lymphocyte stimulator/a proliferation-inducing ligand heterotrimers are elevated in the sera of patients with autoimmune disease and are neutralized by atacicept and B-cell maturation antigen-immunoglobulin. Arthritis Res Ther. 2010; 12(2):R48.
44. Petri M, Stohl W, Chatham W, McCune W J, Chevrier M, Ryel J, et al. Association of plasma B lymphocyte stimulator levels and disease activity in systemic lupus erythematosus. Arthritis Rheum. 2008; 58(8):2453-9.
45. Crowe S R, Merrill J T, Vista E S, Dedeke A B, Thompson D M, Stewart S, et al. Influenza vaccination responses in human systemic lupus erythematosus: impact of clinical and demographic features. Arthritis Rheum. 2011; 63(8):2396-406.
46. Stringer E A, Baker K S, Carroll I R, Montoya J G, Chu L, Maecker H T, et al. Daily cytokine fluctuations, driven by leptin, are associated with fatigue severity in chronic fatigue syndrome: evidence of inflammatory pathology. Journal of translational medicine. 2013; 11(1):93.
47. Dupont N C, Wang K, Wadhwa P D, Culhane J F, Nelson E L. Validation and comparison of luminex multiplex cytokine analysis kits with ELISA: determinations of a panel of nine cytokines in clinical sample culture supematants. J Reprod Immunol. 2005; 66(2):175-91.
48. Dossus L, Becker S, Achaintre D, Kaaks R, Rinaldi S. Validity of multiplex-based assays for cytokine measurements in serum and plasma from “non-diseased” subjects: comparison with ELISA. J Immunol Methods. 2009; 350(1-2):125-32.
49. Munroe M E, Vista E S, Merrill J T, Guthridge J M, Roberts V C, James J A. Pathways of impending disease flare in African-American systemic lupus erythematosus patients. J Autoimmun. 2017; 78:70-8.
50. Munroe M E, Vista E S, Guthridge J M, Thompson L F, Merrill J T, James J A. Pro-inflammatory adaptive cytokines and shed tumor necrosis factor receptors are elevated preceding systemic lupus erythematosus disease flare. Arthritis & rheumatology. 2014; 66(7):1888-99.
51. Genuer R, Poggi J M, Tuleau-Malot C. Variable selection using random forests. Pattern Recogn Lett. 2010; 31(14):2225-36.
52. Chen T, Guestrin C. XGBoost: A Scalable Tree Boosting System. Proceedings of the 22nd acm sigkdd international conference on kowledge discovery and data mining. 2016:785-94.
53. Lu R, Munroe M E, Guthridge J M, Bean K M, Fife D A, Chen H, et al. Dysregulation of Innate and Adaptive Serum Mediators Precedes Systemic Lupus Erythematosus Classification and Improves Prognostic Accuracy of Autoantibodies J Autoimmun. 2016; 74:182-93.
54. Munroe M E, Lu R, Zhao Y D, Fife D A, Robertson J M, Guthridge J M, et al. Altered type II interferon precedes autoantibody accrual and elevated type I interferon activity prior to systemic lupus erythematosus classification. Ann Rheum Dis. 2016; 75(11):2014-21.
55. Okamoto A, Fujio K, Okamura T, Yamamoto K. Regulatory T-cell-associated cytokines in systemic lupus erythematosus. J Biomed Biotechnol. 2011; 2011:463412.
56. Doedens J R, Mahimkar R M, Black R A. TACEIADAM-17 enzymatic activity is increased in response to cellular stimulation. Biochemical and biophysical research communications. 2003; 308(2):331-8.
57. Chen X, Oppenheim J J. The phenotypic and functional consequences of tumour necrosis factor receptor type 2 expression on CD4(+) FoxP3(+) regulatory T cells. Immunology. 2011; 133(4):426-33.
58. van Mierlo G J, Scherer H U, Hameetman M, Morgan M E, Flierman R, Huizinga T W, et al. Cutting edge: TNFR-shedding by CD4+CD25+ regulatory T cells inhibits the induction of inflammatory mediators. J Immunol. 2008; 180(5):2747-51.
59. Mok C C, Ho L Y, Fong L S, To C H. Immunogenicity and safety of a quadrivalent human papillomavirus vaccine in patients with systemic lupus erythematosus: a case-control study. Ann Rheum Dis. 2013; 72(5):659-64.
60. Abu-Shakra M, Zalmanson S, Neumann L, Flusser D, Sukenik S, Buskila D. Influenza virus vaccination of patients with systemic lupus erythematosus: effects on disease activity. J Rheumatol. 2000; 27(7):1681-5.
61. Curtis J R, van der Helm-van Mil A H, Knevel R, Huizinga T W, Haney D J, Shen Y, et al. Validation of a novel multibiomarker test to assess rheumatoid arthritis disease activity. Arthritis care & research. 2012; 64(12):1794-803.
62. Peabody J W, Strand V, Shimkhada R, Lee R, Chemoff D. Impact of rheumatoid arthritis disease activity test on clinical practice. PLoS One. 2013; 8(5):e63215.

Tables

TABLE 1
Demographics and clinical characteristics of SLE patients with Pre-Flare and Pre-SNF longitudinal samples

| | All SLE Patients (n = 90) | | | EA SLE Patients (n = 49) | | | AA SLE Patients (n = 41) | | |
|---|---|---|---|---|---|---|---|---|---|
| | Pre-Flare | Pre-SNF^{a} | | Pre-Flare | Pre-SNF^{a} | | Pre-Flare | Pre-SNF^{a} | |
| | (n = 90) | (n = 90) | p-value^{b} | (n = 49) | (n = 49) | p-value^{b} | (n = 41) | (n = 41) | p-value^{b} |
| Age, mean ± SD years | 43.6 ± 12.4 | 43.8 ± 12.4 | — | 44.0 ± 13.1 | 44.0 ± 13.8 | — | 43.1 ± 11.6 | 43.4 ± 10.8 | — |
| Time from BL to FU, | 100.4 ± 40.1 | 95.8 ± 40.0 | 0.3557 | 103.6 ± 36.6 | 98.1 ± 37.7 | 0.3004 | 96.7 ± 44.1 | 93.1 ± 42.2 | 0.6934 |
| mean ± SD days | | | | | | | | | |

Baseline
Medications: n positive (%)

| Steroids^{c} | 23 | (26%) | 30 | (33%) | 0.3265 | 13 | (27%) | 15 | (31%) | 0.8234 | 10 | (24%) | 15 | (37%) | 0.3374 |
|---|---|---|---|---|---|---|---|---|---|---|---|---|---|---|---|
| Hydroxychloroquine | 65 | (72%) | 64 | (71%) | 1.0000 | 35 | (71%) | 34 | (69%) | 1.0000 | 30 | (73%) | 30 | (73%) | 1.0000 |
| Immunosuppressants^{d} | 27 | (30%) | 26 | (29%) | 1.0000 | 16 | (33%) | 13 | (27%) | 0.6585 | 13 | (32%) | 13 | (32%) | 1.0000 |
| Biologics^{e} | 1 | (1%) | 4 | (4%) | 0.3680 | 1 | (2%) | 1 | (2%) | 1.0000 | 0 | | 3 | (7777%) | 0.2407 |
| Baseline autoantibody | (n = 88) | | (n = 83) | | | (n = 47) | | (n = 44) | | | (n = 41) | | (n = 39) | | |
| specificities: n positive | | | | | | | | | | | | | | | |
| (%)^{f} | | | | | | | | | | | | | | | |
| Anti-dsDNA | 16 | (18%) | 14 | (17%) | 0.8434 | 5 | (11%) | 3 | (7%) | 0.7151 | 11 | (27%) | 11 | (28%) | 1.0000 |
| Anti-chromatin | 25 | (28%) | 26 | (31%) | 0.7391 | 6 | (13%) | 8 | (18%) | 0.5666 | 19 | (46%) | 18 | (46%) | 1.0000 |
| Anti-Ro/SSA | 30 | (34%) | 32 | (39%) | 0.6334 | 18 | (38%) | 18 | (41%) | 0.8328 | 12 | (29%) | 14 | (36%) | 0.6346 |
| Anti-La/SSB | 11 | (13%) | 11 | (13%) | 1.0000 | 8 | (17%) | 7 | (16%) | 1.0000 | 3 | (7%) | 4 | (10%) | 0.7087 |
| Anti-Sm | 17 | (19%) | 16 | (19%) | 1.0000 | 6 | (13%) | 6 | (14%) | 1.0000 | 11 | (27%) | 8 | (21%) | 0.6029 |
| Anti-SmRNP | 28 | (32%) | 25 | (30%) | 0.8693 | 10 | (21%) | 8 | (18%) | 0.7955 | 18 | (44%) | 17 | (44%) | 1.0000 |
| Anti-RNP | 27 | (31%) | 21 | (25%) | 0.4972 | 14 | (30%) | 8 | (18%) | 0.2276 | 13 | (32%) | 13 | (33%) | 1.0000 |
| Baseline # of | 1.8 ± 1.9 | | 1.7 ± 1.8 | | 0.9308 | 1.4 ± 1.8 | | 1.3 ± 1.6 | | 0.7626 | 2.1 ± 2.0 | | 2.2 ± 1.9 | | 0.7537 |
| autoantibody | | | | | | | | | | | | | | | |
| specificities: | | | | | | | | | | | | | | | |
| mean ± SD | | | | | | | | | | | | | | | |
| SELENA-SLEDAI score | 2.8 ± 2.4 | | 2.2 ± 1.8 | | 0.0433 | 2.9 ± 2.6 | | 2.1 ± 1.8 | | 0.0702 | 2.6 ± 2.1 | | 2.3 ± 1.8 | | 0.3632 |
| (at baseline): mean ± SD | | | | | | | | | | | | | | | |
| SELENA-SLEDAI organ | 70 | (78%) | 68 | (76%) | 0.8603 | 39 | (80%) | 35 | (71%) | 0.4815 | 31 | (76%) | 33 | (81%) | 0.7902 |
| system manifestations (at | | | | | | | | | | | | | | | |
| baseline): n positive (%) | | | | | | | | | | | | | | | |
| CNS^{g} | 0 | | 0 | | — | 0 | | 0 | | — | 0 | | 0 | | — |
| Arthritis | 14 | (16%) | 7 | (8%) | 0.1624 | 8 | (16%) | 4 | (8%) | 0.3560 | 6 | (15%) | 3 | (7%) | 0.4821 |
| Renal^{h} | 0 | | 0 | | | 0 | | 0 | | | 0 | | 0 | | |
| Mucocutaneousⁱ | 37 | (41%) | 36 | (40%) | 1.0000 | 21 | (43%) | 19 | (39%) | 0.8373 | 16 | (39%) | 17 | (41%) | 1.0000 |
| Serositis^{j} | 0 | | 0 | | — | 0 | | 0 | | — | 0 | | 0 | | — |
| Serologic^{k} | 33 | (37%) | 30 | (33%) | 0.7548 | 18 | (37%) | 16 | (33%) | 0.8321 | 15 | (37%) | 14 | (34%) | 1.0000 |
| Hematalogic^{l} | 6 | (7%) | 9 | (10%) | 0.5911 | 0 | | 1 | (2%) | 1.0000 | 6 | (15%) | 8 | (20%) | 0.7701 |
| | Flare | | SNF | | | Flare | | SNF | | | Flare | | SNF | | |
| Follow-up | (n = 90) | | (n = 90) | | p-value^{b} | (n = 49) | | (n = 49) | | p-value^{b} | (n = 41) | | (n = 41) | | p-value^{b} |
| SELENA-SLEDAI | 7.3 ± 3.1 | | 1.9 ± 2.0 | | <0.0001 | 7.8 ± 3.5 | | 2.1 ± 2.1 | | <0.0001 | 6.8 ± 2.5 | | 1.8 ± 1.8 | | <0.0001 |
| score (at follow-up): | | | | | | | | | | | | | | | |
| mean ± SD | | | | | | | | | | | | | | | |
| change in SELENA − | 4.6 ± 3.0 | | −0.2 ± 2.0 | | <0.0001 | 4.9 ± 3.5 | | −0.04 ± 2.1 | | <0.0001 | 4.1 ± 2.3 | | −0.6 ± 2.0 | | <0.0001 |
| SLEDAI score (baseline | | | | | | | | | | | | | | | |
| to follow-up): | | | | | | | | | | | | | | | |
| mean ± SD | | | | | | | | | | | | | | | |
| SELENA-SLEDAI | 90 | (100%) | 46 | (58%) | <0.0001 | 49 | (100%) | 26 | (53%) | <0.001 | 41 | (100%) | 20 | (49%) | <0.001 |
| organ system | | | | | | | | | | | | | | | |
| manifestations | | | | | | | | | | | | | | | |
| (at follow-up): | | | | | | | | | | | | | | | |
| n positive (%) | | | | | | | | | | | | | | | |
| CNS^{g} | 2 | (2%) | 0 | | 0.4972 | 2 | (4%) | 0 | | 0.4948 | 0 | | 0 | | — |
| Arthritis | 59 | (66%) | 5 | (6%) | <0.0001 | 36 | (74%) | 3 | (6%) | <0.0001 | 23 | (56%) | 02 | (5%) | <0.0001 |
| Renal^{h} | 0 | | 0 | | | 0 | | 0 | | — | 0 | | 0 | | — |
| Mucocutaneousⁱ | 70 | (78%) | 25 | (28%) | <0.0001 | 40 | (82%) | 14 | (29%) | <0.0001 | 29 | (71%) | 11 | (27%) | 0.0001 |
| Serositis^{j} | 8 | (9%) | 0 | | 0.0066 | 1 | (2%) | 0 | | 1.0000 | 7 | (17%) | 0 | | 0.0118 |
| Serologic^{k} | 38 | (42%) | 33 | (37%) | 0.5420 | 22 | (45%) | 18 | (37%) | 0.5378 | 16 | (39%) | 16 | (39%) | 1.0000 |
| Hematalogic^{l} | 5 | (6%) | 3 | (3%) | 0.7203 | 1 | (2%) | 1 | (2%) | 1.0000 | 4 | (10%) | 2 | (5%) | 0.6755 |

^{a}AA SLE patients with impending disease SELENA-SLEDAI defined disease flare at follow-up vs. the same SLE patients during a comparable period of time without disease flare (self non-flare; SNF)
^{b}Statistical significance (p ≤ 0.05) determined by paired t-test (continuous data) or Fisher's exact test (categorical data)
^{c}Steroids = prednisone, depomedrol
^{d}Immunosuppressants = azathioprine, methotrexate, mycophenolate mofetil
^{e}Biologics = rituximab
^{f}Autoantibody positivity determined by Bioplex 2200 ANA test per manufacturer determined cutoffs
^{g}CNS = seizure, psychosis, organic brain syndrome, visual disturbance, cranial nerve dosirder, lupus headache, CVA
^{h}Renal = urinary casts, hematuria, proteinuria, pyuria
ⁱMucocutaneous = rash, alopecia, mucosal ulcers
^{j}Serositis = pleurisy, pericarditis
^{k}Serologic = low complement, increased DNA binding
^{l}Hematologic = thrombocytopenia, leukopenia

TABLE 2A
Soluble Mediators in Pre-Flare vs. Pre-SNF Longitudinal SLE Samples

| | BL Concentration (pg/ml) | | | | | | | | | |
|---|---|---|---|---|---|---|---|---|---|---|
| | Pre- | | Pre- | | | | BL Mediator vs. FU SELENA-SLEDAI score | | | |
| | Flare | | SNF | | p | q | Spearman | | P | q |
| Analyte | mean | SEM | mean | SEM | value^{a} | value^{b} | r | 95% CI | value^{c} | value^{d} |
| IL-1α | 25.43 | 1.62 | 18.28 | 1.17 | <0.0001 | 0.0013 | 0.2010 | 0.0521 to 0.3412 | 0.0068 | 0.0160 |
| IL-1b | 4.36 | 0.35 | 3.00 | 0.21 | <0.0001 | 0.0013 | 0.1746 | 0.0247 to 0.3168 | 0.0191 | 0.0359 |
| IL-1RA | 913.4 | 66.67 | 1843 | 176.60 | <0.0001 | 0.0013 | −0.3234 | −0.4519 to −0.1817 | <0.0001 | 0.0019 |
| IFN-α | 5.08 | 0.51 | 2.54 | 0.17 | <0.0001 | 0.0013 | 0.2958 | 0.1520 to 0.4273 | <0.0001 | 0.0019 |
| IFN-b | 0.18 | 0.08 | 0.18 | 0.10 | 0.1360 | 0.2128 | 0.0939 | −0.0575 to 0.2410 | 0.2101 | 0.2245 |
| G-CSF | 45.76 | 2.27 | 42.19 | 1.96 | 0.0128 | 0.0284 | 0.0226 | −0.1283 to 0.1726 | 0.7629 | 0.4944 |
| IL-7 | 10.21 | 0.61 | 9.49 | 0.52 | 0.0003 | 0.0020 | 0.0929 | −0.0585 to 0.2400 | 0.2150 | 0.2245 |
| IL-15 | 18.84 | 1.33 | 18.59 | 1.47 | 0.1319 | 0.2128 | 0.0727 | −0.0787 to 0.2208 | 0.3322 | 0.2973 |
| IL-12(p70) | 72.58 | 5.50 | 49.61 | 3.76 | <0.0001 | 0.0013 | 0.2655 | 0.1198 to 0.4001 | 0.0003 | 0.0028 |
| IFN-g | 65.91 | 7.65 | 33.89 | 2.18 | <0.0001 | 0.0013 | 0.3353 | 0.1946 to 0.4625 | <0.0001 | 0.0019 |
| IL-2 | 101.2 | 6.98 | 75.42 | 6.09 | <0.0001 | 0.0013 | 0.2110 | 0.0624 to 0.3504 | 0.0045 | 0.0141 |
| IL-2Rα | 960.5 | 52.17 | 664.6 | 38.27 | <0.0001 | 0.0013 | 0.3423 | 0.2022 to 0.4687 | <0.0001 | 0.0019 |
| IL-6 | 5.08 | 1.27 | 3.64 | 0.44 | 0.2888 | 0.3209 | 0.0865 | −0.0649 to 0.2339 | 0.2485 | 0.2458 |
| IL-23(p19) | 784.4 | 75.3 | 449.4 | 35.88 | <0.0001 | 0.0013 | 0.2266 | 0.0788 to 0.3647 | 0.0022 | 0.0083 |
| IL-17A | 4.27 | 0.40 | 2.35 | 0.25 | <0.0001 | 0.0013 | 0.2049 | 0.0561 to 0.3448 | 0.0058 | 0.0156 |
| IL-21 | 42.02 | 2.30 | 33.92 | 1.88 | <0.0001 | 0.0013 | 0.2347 | 0.0872 to 0.3721 | 0.0015 | 0.0070 |
| IL-4 | 75.85 | 3.39 | 71.84 | 3.04 | <0.0001 | 0.0013 | 0.0959 | −0.0554 to 0.2429 | 0.2004 | 0.2245 |
| IL-5 | 2.26 | 0.21 | 2.13 | 0.21 | 0.4456 | 0.4069 | −0.0033 | −0.1538 to 0.1473 | 0.9647 | 0.5849 |
| IL-13 | 557.1 | 23.37 | 441.4 | 23.57 | <0.0001 | 0.0013 | 0.2575 | 0.1113 to 0.3928 | 0.0005 | 0.0031 |
| IL-10 | 2.85 | 0.79 | 3.28 | 1.54 | 0.0062 | 0.0207 | 0.0781 | −0.0733 to 0.2260 | 0.2973 | 0.2794 |
| TGF-b^{h} | 15.44 | 8.09 | 32.23 | 14.04 | <0.0001 | 0.0013 | −0.3001 | −0.4312 to −0.1567 | <0.0001 | 0.0019 |
| BLyS | 1307 | 92.09 | 1421 | 168.7 | 0.4732 | 0.469 | 0.0256 | −0.1254 to 0.1755 | 0.7326 | 0.4918 |
| APRIL | 4693 | 471.8 | 5368 | 686.7 | 0.2584 | 0.3132 | 0.0003 | −0.1502 to 0.1508 | 0.9967 | 0.5854 |
| CD40L | 1672 | 102.7 | 1031.0 | 56.18 | <0.0001 | 0.0013 | 0.3953 | 0.2603 to 0.5152 | <0.0001 | 0.0019 |
| Fas | 10005 | 334.5 | 7202 | 303.6 | <0.0001 | 0.0013 | 0.4039 | 0.2698 to 0.5226 | <0.0001 | 0.0019 |
| FasL | 61.75 | 2.96 | 36.06 | 2.16 | <0.0001 | 0.0013 | 0.4657 | 0.33910 to 0.5759 | <0.0001 | 0.0019 |
| TNF-α | 12.76 | 0.74 | 10.33 | 0.52 | <0.0001 | 0.0013 | 0.1866 | 0.0361 to 0.3270 | 0.0126 | 0.0263 |
| TNFRI | 2391 | 95.3 | 1306 | 59.59 | <0.0001 | 0.0013 | 0.5297 | 0.4120 to 0.6300 | <0.0001 | 0.0019 |
| TNFRII | 4714 | 291.6 | 2682 | 211.4 | <0.0001 | 0.0013 | 0.4417 | 0.3119 to 0.5553 | <0.0001 | 0.0019 |
| TRAIL | 86.18 | 0.36 | 58.61 | 2.77 | <0.0001 | 0.0013 | 0.3709 | 0.2334 to 0.4939 | <0.0001 | 0.0019 |
| NGF-b | 4.04 | 0.29 | 2.67 | 0.15 | <0.0001 | 0.0013 | 0.2991 | 0.1556 to 0.4302 | <0.0001 | 0.0019 |
| MCP-1/CCL2 | 410.4 | 25.41 | 214.0 | 18.65 | <0.0001 | 0.0013 | 0.5942 | 0.4872 to 0.6836 | <0.0001 | 0.0019 |
| MCP-3/CCL7 | 96.04 | 9.35 | 58.37 | 3.37 | <0.0001 | 0.0013 | 0.3101 | 0.1674 to 0.4401 | <0.0001 | 0.0019 |
| MIP-1α/CCL3 | 290.8 | 13.26 | 183.8 | 7.42 | <0.0001 | 0.0013 | 0.3819 | 0.2455 to 0.5035 | <0.0001 | 0.0019 |
| MIP1-b/CCL4 | 395.9 | 15.16 | 284.9 | 11.57 | <0.0001 | 0.0013 | 0.4005 | 0.2661 to 0.5167 | <0.0001 | 0.0019 |
| RANTES/CCL5 | 4076 | 552.7 | 3948 | 387.6 | 0.7215 | 0.5063 | 0.0325 | −0.1186 to 0.1821 | 0.6654 | 0.4918 |
| Eotaxin/CCL11 | 190.0 | 92.2 | 214.7 | 114.9 | 0.8753 | 0.5632 | 0.0290 | −0.1220 to 0.1788 | 0.6988 | 0.4918 |
| GRO-α/CXCL1 | 150.80 | 9.17 | 150.50 | 9.21 | 0.8870 | 0.5632 | 0.0135 | −0.1373 to 0.1637 | 0.8576 | 0.5373 |
| IL-8/CXCL8 | 6.78 | 1.03 | 5.77 | 0.58 | 0.2425 | 0.3132 | 0.1178 | −0.0334 to 0.2636 | 0.1154 | 0.1581 |
| MIG/CXCL9 | 320.7 | 15.29 | 212.9 | 11.07 | <0.0001 | 0.0013 | 0.3768 | 0.2398 to 0.4990 | <0.0001 | 0.0019 |
| IP-10/CXCL10 | 330.7 | 42.54 | 136.60 | 19.82 | <0.0001 | 0.0013 | 0.5238 | 0.4052 to 0.6250 | <0.0001 | 0.0019 |
| ICAM-1 | 634170 | 44086 | 447193 | 38150 | <0.0001 | 0.0013 | 0.2884 | 0.1441 to 0.4206 | <0.0001 | 0.0019 |
| VCAM-1 | 674811 | 46455 | 626347 | 49217 | 0.0087 | 0.0232 | 0.1170 | −0.0341 to 0.2629 | 0.1178 | 0.1581 |
| E-selectin | 31300 | 1817 | 31337 | 1636 | 0.6918 | 0.5063 | −0.0271 | −0.1769 to 0.1239 | 0.7179 | 0.4918 |
| VEGF | 17.58 | 1.32 | 16.12 | 0.80 | 0.4633 | 0.4069 | 0.1238 | −0.0272 to 0.2693 | 0.0978 | 0.1532 |
| LIF | 1.67 | 0.34 | 1.69 | 0.49 | 0.6280 | 0.4926 | 0.0292 | −0.1219 to 0.1789 | 0.6975 | 0.4918 |
| PAI-1 | 11222 | 1461 | 11331 | 921 | 0.4882 | 0.4069 | −0.0638 | −0.2123 to 0.0876 | 0.3949 | 0.3374 |
| PDGF-BB | 207 | 36.17 | 227.90 | 28.27 | 0.1436 | 0.2128 | −0.0489 | −0.1980 to 0.1024 | 0.5145 | 0.4204 |
| Resistin | 11175 | 952 | 10133 | 822 | 0.0008 | 0.0036 | 0.1307 | −0.0203 to 0.2758 | 0.0804 | 0.1374 |
| Leptin | 45919 | 5536 | 46950 | 6839 | 0.9744 | 0.5906 | −0.1110 | −0.2572 to 0.0402 | 0.1381 | 0.1730 |
| SCF | 88.59 | 3.14 | 47.61 | 2.54 | <0.0001 | 0.0013 | 0.4674 | 0.3408 to 0.5773 | <0.0001 | 0.0019 |

TABLE 2B
LFPI subscore components corresponding to different soluble mediators

| | LFPI subscore Component | | | | | | | |
|---|---|---|---|---|---|---|---|---|
| | Pre- | | Pre- | | | | | |
| | Flare | | SNF | | | | P | q |
| Analyte | median | SD | median | SD | OR^{e} | 95% CI | value^{f} | value^{g} |
| IL-1α | 0.0694 | 0.201 | 0.0360 | 0.200 | 1.64 | 0.86 to 3.05 | 0.1578 | 0.4734 |
| IL-1b | 0.0822 | 0.165 | 0.0613 | 0.183 | 1.07 | 0.54 to 2.15 | 1.0000 | 1.0000 |
| IL-1RA | 0.1597 | 0.296 | −0.1418 | 0.279 | 4.23 | 2.25 to 8.02 | <0.0001 | 0.0039 |
| IFN-α | 0.1000 | 0.276 | 0.0613 | 0.309 | 2.00 | 0.84 to 4.56 | 0.1491 | 0.4734 |
| IFN-b | −0.0378 | 0.102 | −0.0378 | 0.084 | 1.86 | 0.84 to 4.29 | 0.2099 | 0.5847 |
| G-CSF | 0.0032 | 0.022 | 0.0011 | 0.023 | 1.14 | 0.64 to 2.06 | 0.7646 | 0.9332 |
| IL-7 | 0.0322 | 0.096 | 0.0268 | 0.090 | 0.96 | 0.83 to 1.73 | 1.0000 | 1.0000 |
| IL-15 | 0.0179 | 0.073 | 0.0154 | 0.072 | 1.26 | 0.70 to 2.29 | 0.5469 | 0.8597 |
| IL-12(p70) | 0.1169 | 0.209 | 0.0960 | 0.305 | 1.86 | 0.87 to 3.80 | 0.1394 | 0.4734 |
| IFN-g | 0.1537 | 0.305 | 0.0456 | 0.327 | 1.90 | 1.00 to 3.56 | 0.0603 | 0.2352 |
| IL-2 | 0.1147 | 0.204 | 0.0985 | 0.217 | 1.41 | 0.66 to 2.92 | 0.4610 | 0.8597 |
| IL-2Rα | 0.1279 | 0.308 | −0.1217 | 0.330 | 4.66 | 2.46 to 8.59 | <0.0001 | 0.0039 |
| IL-6 | −0.0010 | 0.090 | −0.0014 | 0.083 | 1.14 | 0.64 to 2.05 | 0.7653 | 0.9332 |
| IL-23(p19) | 0.0636 | 0.210 | −0.0358 | 0.225 | 2.17 | 1.20 to 3.84 | 0.0163 | 0.0908 |
| IL-17A | 0.1288 | 0.209 | 0.0808 | 0.200 | 1.33 | 0.67 to 2.51 | 0.5032 | 0.8597 |
| IL-21 | 0.1056 | 0.156 | 0.0651 | 0.290 | 1.27 | 0.68 to 2.29 | 0.5350 | 0.8597 |
| IL-4 | 0.0434 | 0.097 | 0.0347 | 0.095 | 1.00 | 0.54 to 1.84 | 1.0000 | 1.0000 |
| IL-5 | −0.0019 | 0.003 | −0.0019 | 0.003 | 0.90 | 0.48 to 1.68 | 0.8722 | 1.0000 |
| IL-13 | 0.1098 | 0.217 | −0.0067 | 0.277 | 2.60 | 1.39 to 4.86 | 0.0035 | 0.0416 |
| IL-10 | 0.0442 | 0.076 | 0.0430 | 0.080 | 1.29 | 0.70 to 2.40 | 0.5255 | 0.8597 |
| TGF-b^{h} | −0.0206 | 0.309 | −0.1962 | 0.256 | 4.42 | 2.25 to 8.52 | <0.0001 | 0.0039 |
| BLyS | −0.0045 | 0.024 | −0.0048 | 0.027 | 1.26 | 0.69 to 2.30 | 0.5455 | 0.8597 |
| APRIL | 0.0001 | 0.0003 | 0.0001 | 0.0003 | 1.09 | 0.48 to 2.49 | 1.0000 | 1.0000 |
| CD40L | 0.1226 | 0.172 | −0.0308 | 0.513 | 3.99 | 2.09 to 7.26 | <0.0001 | 0.0039 |
| Fas | 0.1661 | 0.263 | −0.0968 | 0.456 | 4.28 | 2.26 to 7.89 | <0.0001 | 0.0039 |
| FasL | 0.2589 | 0.407 | −0.1085 | 0.452 | 6.05 | 3.05 to 11.7 | <0.0001 | 0.0039 |
| TNF-α | 0.0485 | 0.221 | 0.0098 | 0.142 | 1.38 | 0.76 to 2.53 | 0.3644 | 0.8041 |
| TNFRI | 0.2956 | 0.380 | −0.2807 | 0.488 | 10.20 | 5.14 to 19.6 | <0.0001 | 0.0039 |
| TNFRII | 0.2198 | 0.332 | −0.1524 | 0.461 | 9.10 | 4.65 to 17.2 | <0.0001 | 0.0039 |
| TRAIL | 0.2005 | 0.349 | −0.1677 | 0.337 | 4.69 | 2.47 to 8.54 | <0.0001 | 0.0039 |
| NGF-b | 0.0917 | 0.316 | −0.0546 | 0.253 | 4.42 | 2.35 to 8.41 | <0.0001 | 0.0039 |
| MCP-1/CCL2 | 0.3353 | 0.482 | −0.3497 | 0.496 | 14.10 | 6.74 to 29.6 | <0.0001 | 0.0039 |
| MCP-3/CCL7 | 0.0811 | 0.148 | −0.0006 | 0.040 | 2.97 | 1.59 to 5.57 | 0.0008 | 0.0156 |
| MIP-1α/CCL3 | 0.1170 | 0.324 | 0.0473 | 0.427 | 3.27 | 1.45 to 7.85 | 0.0064 | 0.0416 |
| MIP1-b/CCL4 | 0.2185 | 0.356 | −0.0067 | 0.387 | 2.46 | 1.32 to 4.55 | 0.0059 | 0.0416 |
| RANTES/CCL5 | −0.0017 | 0.035 | 0.0013 | 0.030 | 0.84 | 0.47 to 1.49 | 0.6548 | 0.9120 |
| Eotaxin/CCL11 | −0.00003 | 0.029 | 0.0005 | 0.029 | 0.91 | 0.51 to 1.63 | 0.8815 | 1.0000 |
| GRO-α/CXCL1 | 0.0036 | 0.014 | 0.0036 | 0.014 | 0.58 | 0.25 to 1.31 | 0.2890 | 0.7514 |
| IL-8/CXCL8 | 0.0090 | 0.145 | −0.0064 | 0.083 | 1.37 | 0.77 to 2.46 | 0.3711 | 0.8041 |
| MIG/CXCL9 | 0.1195 | 0.264 | 0.0848 | 0.454 | 2.59 | 1.16 to 5.71 | 0.0309 | 0.1506 |
| IP-10/CXCL10 | 0.1930 | 0.380 | −0.2684 | 0.543 | 2.54 | 1.81 to 3.68 | <0.0001 | 0.0039 |
| ICAM-1 | 0.1204 | 0.278 | 0.0180 | 0.290 | 2.63 | 1.42 to 4.89 | 0.0044 | 0.0416 |
| VCAM-1 | 0.0027 | 0.113 | −0.0203 | 0.121 | 1.20 | 0.67 to 2.14 | 0.6548 | 0.9120 |
| E-selectin | 0.0049 | 0.029 | 0.0023 | 0.026 | 1.14 | 0.64 to 2.06 | 07650 | 0.9332 |
| VEGF | 0.0105 | 0.135 | 0.0114 | 0.112 | 1.00 | 0.56 to 1.80 | 1.0000 | 1.0000 |
| LIF | −0.0210 | 0.030 | −0.0210 | 0.029 | 1.41 | 0.75 to 2.58 | 0.3485 | 0.8041 |
| PAI-1 | 0.0050 | 0.063 | −0.0050 | 0.065 | 1.25 | 0.70 to 2.24 | 0.5511 | 0.8597 |
| PDGF-BB | 0.0005 | 0.039 | −0.0091 | 0.058 | 1.20 | 0.67 to 2.15 | 0.6545 | 0.9120 |
| Resistin | 0.0184 | 0.093 | −0.0103 | 0.159 | 1.87 | 1.05 to 3.44 | 0.0523 | 0.2266 |
| Leptin | 0.0057 | 0.111 | −0.0045 | 0.111 | 1.14 | 0.64 to 2.05 | 0.7657 | 0.9332 |
| SCF | 0.3140 | 0.2900 | −0.2701 | 0.4290 | 14.30 | 6.79 to 28.6 | <0.0001 | 0.0039 |

^{a}Wilcoxon matched pairs test; significant values (p ≤ 0.05) in bold
^{b}Benjamini-Hochberg multiple testing procedure (False Discovery Rate ≤ 0.05) using R version 2.15.3; significant values (q ≤ 0.05) in bold
^{c}Spearman rank correlation; significant values (p ≤ 0.05) in bold
^{d}Benjamini-Hochberg multiple testing procedure (False Discovery Rate ≤ 0.05 ) using R version 2.15.3
^{e}Odds Ratio (# of Pre-Flare vs Pre-SNF SLE patients with positive or negative LFPI subscore component value)
^{f}Fisher's exact test; significant values (p ≤ 0.05) in bold
^{g}Benjamini-Hochberg multiple testing procedure (False Discovery Rate ≤ 0.05) using R version 2.15.3; significant values (q ≤ 0.05) in bold
^{h}Active TGF-beta measured as part of multiplex xMAP assay using Ab pair (BR19) used in previous publications
BL = Baseline;
FU = Follow-up

TABLE 3A
Random Forest Ranking of Informative Soluble Mediators Altered Prior to Clinical Disease Flare

| | | BL Concentration (pg/ml) | | | | | | | |
|---|---|---|---|---|---|---|---|---|---|
| | | Pre- | | Pre- | | | BL Mediator vs. FU hSLEDAI score | | |
| RF | | Flare | | SNF | | | Spearman | | |
| Rank | Analyte | mean | SEM | mean | SEM | p value^{a} | r | 95% CI | P value^{b} |
| 1 | SCF | 88.59 | 3.14 | 47.61 | 2.54 | <0.0001 | 0.4674 | 0.3408 to 0.5773 | <0.0001 |
| 2 | MCP-1/CCL2 | 410.4 | 25.41 | 214.0 | 18.65 | <0.0001 | 0.5942 | 0.4872 to 0.6836 | <0.0001 |
| 3 | TNFRI | 2391 | 95.3 | 1306 | 59.59 | <0.0001 | 0.5297 | 0.4120 to 0.6300 | <0.0001 |
| 4 | IL-1RA | 913.4 | 66.67 | 1843 | 176.60 | <0.0001 | −0.3234 | −0.4519 to −0.1817 | <0.0001 |
| 5 | MIP-1α/CCL3 | 290.8 | 13.26 | 183.8 | 7.42 | <0.0001 | 0.3819 | 0.2455 to 0.5035 | <0.0001 |
| 6 | TNFRII | 4714 | 291.6 | 2682 | 211.4 | <0.0001 | 0.4417 | 0.3119 to 0.5553 | <0.0001 |
| 7 | IP-10/CXCL10 | 330.7 | 42.54 | 136.60 | 19.82 | <0.0001 | 0.5238 | 0.4052 to 0.6250 | <0.0001 |
| 8 | TGF-b (native) | 15.44 | 8.09 | 32.23 | 14.04 | <0.0001 | −0.3001 | −0.4312 to −0.1567 | <0.0001 |
| 9 | MIG/CXCL9 | 320.7 | 15.29 | 212.9 | 11.07 | <0.0001 | 0.3768 | 0.2398 to 0.4990 | <0.0001 |
| 10 | IFN-g | 65.91 | 7.65 | 33.89 | 2.18 | <0.0001 | 0.3353 | 0.1946 to 0.4625 | <0.0001 |
| 11 | TRAIL | 86.18 | 0.36 | 58.61 | 2.77 | <0.0001 | 0.3709 | 0.2334 to 0.4939 | <0.0001 |
| 12 | MIP1-b/CCL4 | 395.9 | 15.16 | 284.9 | 11.57 | <0.0001 | 0.4005 | 0.2661 to 0.5167 | <0.0001 |
| 13 | ICAM-1 | 634170 | 44086 | 447193 | 38150 | <0.0001 | 0.2884 | 0.1441 to 0.4206 | <0.0001 |
| 14 | TGF-b (total) | 16692 | 1984 | 26331 | 2630 | <0.0001 | −0.2439 | −0.3804 to −0.0969 | 0.0010 |

TABLE 3B
LFPI score component of Random Forest Ranked Soluble Mediators

| | | LFPI Score Component | | | | | | |
|---|---|---|---|---|---|---|---|---|
| | | Pre- | | Pre- | | | | |
| RF | | Flare | | SNF | | | | |
| Rank | Analyte | median | SD | median | SD | OR^{c} | 95% CI | P value^{d} |
| 1 | SCF | 0.3140 | 0.2900 | −0.2701 | 0.4290 | 14.30 | 6.79 to 28.6 | <0.0001 |
| 2 | MCP-1/CCL2 | 0.3353 | 0.482 | −0.3497 | 0.496 | 14.10 | 6.74 to 29.6 | <0.0001 |
| 3 | TNFRI | 0.2956 | 0.380 | −0.2807 | 0.488 | 10.20 | 5.14 to 19.6 | <0.0001 |
| 4 | IL-1RA | 0.1597 | 0.296 | −0.1418 | 0.279 | 4.23 | 2.25 to 8.02 | <0.0001 |
| 5 | MIP-1α/CCL3 | 0.1170 | 0.324 | 0.0473 | 0.427 | 3.27 | 1.45 to 7.85 | 0.0064 |
| 6 | TNFRII | 0.2198 | 0.332 | −0.1524 | 0.461 | 9.10 | 4.65 to 17.2 | <0.0001 |
| 7 | IP-10/CXCL10 | 0.1930 | 0.380 | −0.2684 | 0.543 | 2.54 | 1.81 to 3.68 | <0.0001 |
| 8 | TGF-b (native) | −0.0206 | 0.309 | −0.1962 | 0.256 | 4.42 | 2.25 to 8.52 | <0.0001 |
| 9 | MIG/CXCL9 | 0.1195 | 0.264 | 0.0848 | 0.454 | 2.59 | 1.16 to 5.71 | 0.0309 |
| 10 | IFN-g | 0.1537 | 0.305 | 0.0456 | 0.327 | 1.90 | 1.00 to 3.56 | 0.0603 |
| 11 | TRAIL | 0.2005 | 0.349 | −0.1677 | 0.337 | 4.69 | 2.47 to 8.54 | <0.0001 |
| 12 | MIP1-b/CCL4 | 0.2185 | 0.356 | −0.0067 | 0.387 | 2.46 | 1.32 to 4.55 | 0.0059 |
| 13 | ICAM-1 | 0.1204 | 0.278 | 0.0180 | 0.290 | 2.63 | 1.42 to 4.89 | 0.0044 |
| 14 | TGF-b (total) | 0.0869 | 0.228 | −0.0727 | 0.2387 | 3.87 | 2.06 to 7.31 | <0.0001 |

BL = Baseline;
FU = Follow-up;
hSLEDAI = hybrid SLEDAI;
LFPI = Lupus Flare Prediction Index;
RF = Random Forest;
SNF = SelfNon-flare
^{a}Wilcoxon matched pairs test; Bonferonni corrected significant p = 0.0035 in bold
^{b}Spearman rank correlation; Bonferonni corrected significant p = 0.0035 in bold
^{c}Odds Ratio (# of Pre-Flare vs Pre-SNF SLE patients with positive or negative LFPI component value)
^{d}Fisher's exact test; Bonferonni corrected significant p = 0.0035 in bold

TABLE 4
Biomarker/Soluble Mediators and UnitProt Identifier

| | | UnitProt |
|---|---|---|
| Analyte | Protein Name | Identifier |
| IL-1α | Interleukin-1 alpha | P01583 |
| IL-1b | Interleukin-1 beta | P01584 |
| IL-1RA | Interleukin-1 receptor antagonist | P18510 |
| | protein | |
| IFN-α | Interferon alpha-1 | P01562 |
| IFN-b | Interferon beta | P01574 |
| G-CSF | Granulocyte colony-stimulating | Q99062 |
| | factor receptor | |
| IL-7 | Interleukin-7 | P13232 |
| IL-15 | Interleukin-15 | P40933 |
| IL-12 (p70) | Interleukin 12 (p70) | P29459 |
| IFN-γ | Interferon gamma | P01579 |
| IL-2 | Interleukin-2 | P60568 |
| IL-2R_{α} | Interleukin-2 receptor subunit | P01589 |
| | alpha | |
| IL-6 | Interleukin-6 | P05231 |
| IL-23 (p19) | Interleukin-23 subunit alpha | Q9NPF7 |
| IL-17A | Interleukin-17A | Q16552 |
| IL-21 | Interleukin-21 | Q9HBE4 |
| IL-4 | Interleukin-4 | P05112 |
| IL-5 | Interleukin-5 | P05113 |
| IL-13 | Interleukin-13 | P35225 |
| IL-10 | Interleukin-10 | P22301 |
| TGF-β | Transforming Growth Factor Beta | P01137 |
| BLyS | Tumor necrosis factor ligand | Q9Y275 |
| | superfamily member 13B | |
| APRIL | Tumor necrosis factor ligand | Q9D777 |
| | superfamily member 13 | |
| CD40L | CD40 ligand | P29965 |
| Fas | Tumor necrosis factor receptor | P25445 |
| | superfamily member 6 | |
| FasL | Tumor necrosis factor ligand | P48023 |
| | superfamily member 6 | |
| TNF-α | Tumor necrosis factor | P01375 |
| TNFRI | Tumor necrosis factor receptor | P19438 |
| | superfamily member 1A | |
| TNFRII | Tumor necrosis factor receptor | P20333 |
| | superfamily member 1B | |
| TRAIL | TNF-related apoptosis-inducing | P50591 |
| | ligand | |
| NGF-β | Beta-nerve growth factor | P01138 |
| MCP-1/CCL2 | C—C motif chemokine 2 | P13500 |
| MCP-3/CCL7 | C—C motif chemokine 7 | P80098 |
| MIP-1α/CCL3 | C—C motif chemokine 3 | P10147 |
| MIP1-b/CCL4 | C—C motif chemokine 4 | P13236 |
| RANTES/CCL5 | C—C motif chemokine 5 | P13501 |
| Eotaxin/CCL11 | C—C motif chemokine 11 | P51671 |
| GRO-α/CXCL1 | Growth-regulated alpha protein | P09341 |
| IL-8/CXCL8 | Interleukin-8 | P10145 |
| MIG/CXCL9 | C—X—C motif chemokine 9 | Q07325 |
| IP-10/CXCL10 | C—X—C motif chemokine 10 | P02778 |
| ICAM-1 | Intercellular adhesion molecule 1 | P05362 |
| VCAM-1 | Vascular cell adhesion protein 1 | P19320 |
| E-selectin | E-selectin | P16581 |
| VEGF | Vascular endothelial growth | P15692 |
| | factor A | |
| LIF | Leukemia inhibitory factor | P15018 |
| PAI-1 | Plasminogen activator inibitor 1 | P05121 |
| PDGF-BB | Platelet-derived growth factor | P01127 |
| | subunit B | |
| Resistin | Resistin | Q9HD89 |
| Leptin | Leptin | P41159 |
| SCF | Kit ligand | P21583 |

1. A method comprising:
(a) obtaining a dataset comprising expression levels of biomarkers from a test sample from a systemic lupus erythematosus (SLE) subject, wherein the biomarkers comprise:
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
(b) generating a Lupus Flare Predictive Index (LFPI) based on the expression levels in the obtained dataset; and
(c) determining likelihood of a future SLE disease activity event in the SLE subject based on the LFPI.

2. The method of claim 1, wherein
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG),
the at least two TNFR superfamily member molecules comprise tumor necrosis factor receptor I (TNFRI), tumor necrosis factor receptor II (TNFRII),
the at least two regulatory mediator molecules comprise native transforming growth factor beta (native TGF-β) and an interleukin-1 receptor antagonist (IL-1RA), and
the at least one SLE mediator molecule comprises stem cell factor (SCF).

3. The method of claim 1 or 2, wherein the biomarkers further comprise at least one T-helper type-1 (Th1) cytokines selected from interferon-gamma (IFN-γ), LL-12p70, IL-2, and IL-2R_{α}.

4. The method of claim 3, wherein the at least one Th1 cytokine comprises interferon-gamma (IFN-γ).

5. The method of claim 1, wherein:
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1);
the at least two TNFR superfamily member molecules comprise tumor necrosis factor receptor I (TNFRI), tumor necrosis factor receptor II (TNFRII), and tumor necrosis factor-related apoptosis-inducing ligand (TRAIL),
the at least two regulatory mediator molecules comprise native transforming growth factor beta (native TGF-β), an interleukin-1 receptor antagonist (IL-1RA), a total transforming growth factor beta (total TGF-β), and
the at least one SLE mediator molecule comprises stem cell factor (SCF), and
wherein the biomarkers further comprise one or more T-helper type-1 (Th1) cytokines, wherein the one or more Th1 cytokines comprise an interferon-gamma (IFN-γ).

6. A method comprising:
(a) obtaining a dataset comprising expression levels of biomarkers from a test sample from a systemic lupus erythematosus (SLE) subject, wherein the biomarkers comprise:
at least one innate cytokine selected from IL-7, IL-1α, and IL-10;
at least one Th1 cytokine selected from interferon-gamma (IFN-γ), IL-12p70, IL-2, and IL-2R_{α};
at least one Th2 cytokine selected from IL-4 and IL-13;
at least one Th17 cytokine selected from IL-17A, IL-6, IL-21, and IL-23;
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
(b) generating a LFPI based on the expression levels in the obtained dataset; and
(c) determining likelihood of a future SLE disease activity event in the SLE subject based on the LFPI.

7. A method comprising:
(a) obtaining or having obtained a dataset comprising expression levels of biomarkers from a test sample from a systemic lupus erythematosus (SLE) subject, wherein the biomarkers comprise:
(i) chemokine(s) or adhesion molecules, wherein the chemokine(s) or adhesion molecules comprise:
a C—C motif chemokine ligand 2 (CCL2) monocyte chemoattractant protein-1 (MCP-1),
a C—C motif chemokine ligand 3 (CCL3)/macrophage inflammatory protein-1 alpha (MIP-1α),
a C—X—C motif chemokine ligand 10 (CXCL10)/IFN-gamma-inducible protein 10 (IP-10), and
a C—X—C motif chemokine ligand 9 (CXCL9)/monokine induced by interferon-gamma (MIG);
(ii) tumor necrosis factor receptor (TNFR) superfamily member molecules,
wherein the TNFR superfamily member molecules comprise:
a tumor necrosis factor receptor I (TNFRI), and
a tumor necrosis factor receptor II (TNFRII);
(iii) regulatory mediator molecules, wherein the regulatory mediator molecules comprise:
native transforming growth factor beta (native TGF-β), and
an interleukin-1 receptor antagonist (IL-1RA); and
(iv) one or more systemic lupus erythematosus (SLE) mediator molecules, wherein the one or more SLE mediator molecules comprise a stem cell factor (SCF);
(b) generating a LFPI based on the expression levels in the obtained dataset; and
(c) determining likelihood of a future SLE disease activity event in the SLE subject based on the LFPI.

8. The method of claim 7, wherein the biomarkers further comprise one or more T-helper type-1 (Th1) cytokines, wherein the one or more Th1 cytokines comprise an interferon-gamma (IFN-γ).

9. The method of claim 7 or 8, wherein the tumor necrosis factor receptor (TNFR) superfamily member molecules further comprise a tumor necrosis factor-related apoptosis-inducing ligand (TRAIL).

10. The method of any one of claims 7-9, wherein the chemokine(s) or adhesion molecules further comprise:
a C—C motif chemokine ligand 4 (CCL4)/macrophage inflammatory protein-1 beta (MIP-1β) and an Intercellular Adhesion Molecule 1 (ICAM-1).

11. The method of any one of claims 7-10, wherein the regulatory mediator molecules further comprise total TGF-β.

12. The method of any one of claims 7-11, wherein:
the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1);
the TNFR superfamily member molecules further comprise a tumor necrosis factor-related apoptosis-inducing ligand (TRAIL); and
the regulatory mediator molecules further comprise a total transforming growth factor beta (total TGF-β).

13. The method of any one of claims 7-12, wherein the biomarkers further comprise (vi) one or more innate cytokines, wherein the one or more innate cytokines are selected from IL-7, IL-1α, and IL-1β.

14. The method of any one of claims 7-13, wherein the biomarkers further comprise (vii) one or more T-helper type-2 (Th2) cytokines, wherein the one or more Th2 cytokines are selected from IL-13 and IL-4.

15. The method of any one of claims 7-14, wherein the biomarkers further comprise (viii) one or more Th17 cytokines, wherein the one or more Th17 cytokines comprise IL-17A.

16. The method of any one of claims 7-15, wherein the one or more SLE mediator molecules further comprise Resistin.

17. The method of any one of claims 8-16, wherein the Th1 cytokines further comprise IL-2, IL-12p70, and IL-2R_{α}.

18. The method of any one of claims 7-17, wherein the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

19. The method of any one of claims 7-18, wherein:
the Th1 cytokines comprise interferon-gamma (IFN-γ), IL-2R_{α}, IL-12p70, and IL-2;
the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8;
the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α;
the regulatory mediator molecules comprise: native transforming growth factor beta (native TGF-β), an interleukin-1 receptor antagonist (IL-1RA), a total transforming growth factor beta (total TGF-β), and IL-10;
the SLE mediator molecules comprise: a stem cell factor (SCF) and Resistin; and
wherein the biomarkers further comprise:
innate cytokines, wherein the innate cytokines comprise: IL-7, IL-1α, and IL-1β;
T-helper type-2 (Th2) cytokines, wherein the Th2 cytokines comprise: IL-13 and IL-4; and
one or more Th17 cytokines, wherein the one or more Th17 cytokines comprise IL-17A.

20. The method of any one of the preceding claims, wherein the expression levels of biomarkers comprise protein levels.

21. The method of claim 20, wherein the expression levels of biomarkers are determined using one of an ELISA assay, xMAP® technology, or SimplePlex™ assay.

22. The method of any one of the preceding claims, wherein the expression levels of biomarkers comprise mRNA levels.

23. The method of claim 22, wherein the mRNA levels are obtained from circulating cells.

24. The method of claim 22, wherein the mRNA levels are obtained from circulating T-cells.

25. The method of any one of the preceding claims, wherein generating the LFPI based on the expression levels comprises applying a predictive model.

26. The method of claim 25, wherein applying the predictive model comprises, for the expression level of each biomarker:
log-transforming the expression level;
standardizing the expression level;
obtaining a corresponding coefficient for the biomarker; the corresponding coefficient representing an association between pre-flare expression levels of the biomarker and a measurement of SLE clinical disease activity; and
weighting the standardized expression level with the corresponding coefficient to obtain a LFPI subscore for the biomarker; and
summing the LFPI subscores to obtain the LFPI.

27. The method of claim 26, wherein for each expression level, the corresponding coefficient is obtained from a linear regression model testing associations between the measurement of SLE clinical disease activity and the pre-flare expression levels of the biomarker.

28. The method of claim 26 or 27, wherein standardizing the expression level comprises normalizing the expression level to a mean expression value for SLE patients with stable SLE disease.

29. The method of any one of claims 25-28, wherein the biomarkers were selected for inclusion in the dataset using an applied machine learning modeling approach.

30. The method of claim 29, wherein the applied machine learning modeling approach is one of random forest or gradient boosting.

31. The method of any one of claims 26-30, wherein the measurement of SLE clinical disease activity is the Safety of Estrogens in Lupus Erythematosus National Assessment-Systemic Lupus Erythematosus Disease Activity Index (SELENA-SLEDAI).

32. The method of any one of claims 26-31, wherein the measurement of SLE clinical disease activity is determined from samples obtained from a group of patients undergoing a flare event.

33. The method of any one of claims 25-32, wherein performance of the predictive model is characterized by an area under a receiver operating characteristic curve that is greater than 0.85.

34. The method of any one of claims 25-32, wherein performance of the predictive model is characterized by an area under a receiver operating characteristic curve that is greater than 0.90.

35. The method of any one of the preceding claims, further comprising administering a treatment to the SLE subject.

36. The method of any one of the preceding claims, wherein obtaining the dataset comprising expression levels of biomarkers comprises:
obtaining a blood, serum or plasma sample from the SLE subject; and
assessing expression levels of biomarkers from the test sample from the SLE subject.

37. The method of any one of the preceding claims, wherein the future SLE disease activity event is one of a future flare event or future organ damage.

38. The method of any one of the preceding claims, wherein the dataset further comprises expression levels of biomarkers from a second test sample taken from the systemic lupus erythematosus (SLE) subject at a different time point.

39. A non-transitory computer readable medium storing instructions that, when executed by a processor, cause the processor to perform the steps of:
(a) obtaining a dataset comprising expression levels of biomarkers from a test sample from a systemic lupus erythematosus (SLE) subject, wherein the biomarkers comprise:
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
(b) generating a LFPI based on the expression levels in the obtained dataset; and
(c) determining likelihood of a future SLE disease activity event in the SLE subject based on the LFPI.

40. The non-transitory computer readable medium of claim 39, wherein:
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG),
the at least two TNFR superfamily member molecules comprise tumor necrosis factor receptor I (TNFRI), tumor necrosis factor receptor II (TNFRII),
the at least two regulatory mediator molecules comprise native transforming growth factor beta (native TGF-β) and an interleukin-1 receptor antagonist (IL-1RA), and
the at least one SLE mediator molecule comprises stem cell factor (SCF).

41. The non-transitory computer readable medium of claim 39 or 40, wherein the biomarkers further comprise at least one T-helper type-1 (Th1) cytokines selected from interferon-gamma (IFN-γ), IL-12p70, IL-2, and IL-2R_{α}.

42. The non-transitory computer readable medium of claim 41, wherein the at least one Th1 cytokine comprises interferon-gamma (IFN-γ).

43. The non-transitory computer readable medium of claim 39, wherein:
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1);
the at least two TNFR superfamily member molecules comprise tumor necrosis factor receptor I (TNFRI), tumor necrosis factor receptor II (TNFRII), and tumor necrosis factor-related apoptosis-inducing ligand (TRAIL),
the at least two regulatory mediator molecules comprise native transforming growth factor beta (native TGF-β), an interleukin-1 receptor antagonist (IL-1RA), a total transforming growth factor beta (total TGF-β), and
the at least one SLE mediator molecule comprises stem cell factor (SCF), and
wherein the biomarkers further comprise one or more T-helper type-1 (Th1) cytokines, wherein the one or more Th1 cytokines comprise an interferon-gamma (IFN-γ).

44. A non-transitory computer readable medium storing instructions that, when executed by a processor, cause the processor to perform the steps of:
(a) obtaining a dataset comprising expression levels of biomarkers from a test sample from a systemic lupus erythematosus (SLE) subject, wherein the biomarkers comprise:
at least one innate cytokine selected from IL-7, IL-1α, and IL-1β;
at least one Th1 cytokine selected from interferon-gamma (IFN-γ), IL-12p70, IL-2, and IL-2R_{α};
at least one Th2 cytokine selected from IL-4 and IL-13;
at least one Th17 cytokine selected from IL-17A, IL-6, IL-21, and IL-23;
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
(b) generating a LFPI based on the expression levels in the obtained dataset; and
(c) determining likelihood of a future SLE disease activity event in the SLE subject based on the LFPI.

45. A non-transitory computer readable medium storing instructions that, when executed by a processor, cause the processor to perform the steps of:
(a) obtaining a dataset comprising expression levels of biomarkers from a test sample from a systemic lupus erythematosus (SLE) subject, wherein the biomarkers comprise:
(i) chemokine(s) or adhesion molecules, wherein the chemokine(s) or adhesion molecules comprise:
a C—C motif chemokine ligand 2 (CCL2)/monocyte chemoattractant protein-1 (MCP-1),
a C—C motif chemokine ligand 3 (CCL3)/macrophage inflammatory protein-1 alpha (MIP-1α),
a C—X—C motif chemokine ligand 10 (CXCL10)/IFN-gamma-inducible protein 10 (IP-10), and
a C—X—C motif chemokine ligand 9 (CXCL9)/monokine induced by interferon-gamma (MIG);
(ii) tumor necrosis factor receptor (TNFR) superfamily member molecules, wherein the TNFR superfamily member molecules comprise:
a tumor necrosis factor receptor I (TNFRI), and
a tumor necrosis factor receptor II (TNFRII);
(iii) regulatory mediator molecules, wherein the regulatory mediator molecules comprise:
native transforming growth factor beta (native TGF-β), and
an interleukin-1 receptor antagonist (IL-1RA); and
(iv) one or more systemic lupus erythematosus (SLE) mediator molecules, wherein the one or more SLE mediator molecules comprise a stem cell factor (SCF);
(b) generating a LFPI based on the expression levels in the obtained dataset; and
(c) determining likelihood of a future SLE disease activity event in the SLE subject based on the LFPI.

46. The non-transitory computer readable medium of claim 45, wherein the biomarkers further comprise one or more T-helper type-1 (Th1) cytokines, wherein the one or more Th1 cytokines comprise an interferon-gamma (IFN-γ).

47. The non-transitory computer readable medium of claim 45 or 46, wherein the tumor necrosis factor receptor (TNFR) superfamily member molecules further comprise a tumor necrosis factor-related apoptosis-inducing ligand (TRAIL).

48. The non-transitory computer readable medium of any one of claims 45-47, wherein the chemokine(s) or adhesion molecules further comprise:
a C—C motif chemokine ligand 4 (CCL4)/macrophage inflammatory protein-1 beta (MIP-1β) and an Intercellular Adhesion Molecule 1 (ICAM-1).

49. The non-transitory computer readable medium of any one of claims 45-48, wherein the regulatory mediator molecules further comprise total TGF-β.

50. The non-transitory computer readable medium of any one of claims 45-49, wherein:
the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1);
the TNFR superfamily member molecules further comprise a tumor necrosis factor-related apoptosis-inducing ligand (TRAIL); and
the regulatory mediator molecules further comprise a total transforming growth factor beta (total TGF-β).

51. The non-transitory computer readable medium of any one of claims 45-50, wherein the biomarkers further comprise (vi) one or more innate cytokines, wherein the one or more innate cytokines are selected from IL-7, IL-1α, and IL-1β.

52. The non-transitory computer readable medium of any one of claims 45-51, wherein the biomarkers further comprise (vii) one or more T-helper type-2 (Th2) cytokines, wherein the one or more Th2 cytokines are selected from IL-13 and IL-4.

53. The non-transitory computer readable medium of any one of claims 45-52, wherein the biomarkers further comprise (viii) one or more Th17 cytokines, wherein the one or more Th17 cytokines comprise IL-17A.

54. The non-transitory computer readable medium of any one of claims 45-53, wherein the one or more SLE mediator molecules further comprise Resistin.

55. The non-transitory computer readable medium of any one of claims 46-54, wherein the Th1 cytokines further comprise IL-2, IL-12p70, and IL-2R_{α}.

56. The non-transitory computer readable medium of any one of claims 45-55, wherein the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

57. The non-transitory computer readable medium of any one of claims 45-56, wherein:
the Th1 cytokines comprise interferon-gamma (IFN-γ), LL-2R_{α}, IL-12p70, and IL-2;
the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8;
the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α;
the regulatory mediator molecules comprise: native transforming growth factor beta (native TGF-β), an interleukin-1 receptor antagonist (IL-1RA), a total transforming growth factor beta (total TGF-β), and IL-10;
the SLE mediator molecules comprise: a stem cell factor (SCF) and Resistin; and
wherein the biomarkers further comprise:
innate cytokines, wherein the innate cytokines comprise: IL-7, IL-1α, and IL-1β;
T-helper type-2 (Th2) cytokines, wherein the Th2 cytokines comprise: IL-13 and IL-4; and
one or more Th17 cytokines, wherein the one or more Th17 cytokines comprise IL-17A.

58. The non-transitory computer readable medium of any one of claims 39-57, wherein the expression levels of biomarkers comprise protein levels.

59. The non-transitory computer readable medium of claim 58, wherein the expression levels of biomarkers are determined using one of an ELISA assay, xMAP® technology, or SimplePlex™ assay.

60. The non-transitory computer readable medium of any one of claims 39-57, wherein the expression levels of biomarkers comprise mRNA levels.

61. The non-transitory computer readable medium of claim 60, wherein the mRNA levels are obtained from circulating cells.

62. The non-transitory computer readable medium of claim 60, wherein the mRNA levels are obtained from circulating T-cells.

63. The non-transitory computer readable medium of any one of claims 39-62, wherein the instructions that cause the processor to perform the step of generating the LFPI based on the expression levels comprise instructions that, when executed by the processor, cause the processor to perform the step of applying a predictive model.

64. The non-transitory computer readable medium of claim 63, wherein the instructions that cause the processor to perform the step of applying the predictive model comprise instructions that, when executed by the processor, cause the processor to perform the steps of:
for the expression level of each biomarker:
log-transforming the expression level;
standardizing the expression level;
obtaining a corresponding coefficient for the biomarker; the corresponding coefficient representing an association between pre-flare expression levels of the biomarker and a measurement of SLE clinical disease activity; and
weighting the standardized expression level with the corresponding coefficient to obtain a LFPI subscore for the biomarker; and
summing the LFPI subscores to obtain the LFPI.

65. The non-transitory computer readable medium of claim 64, wherein for each expression level, the corresponding coefficient is obtained from a linear regression model testing associations between the measurement of SLE clinical disease activity and the pre-flare expression levels of the biomarker.

66. The non-transitory computer readable medium of claim 64 or 65, wherein the instructions that cause the processor to perform the step of standardizing the expression level further comprises instructions that, when executed by the processor, cause the processor to perform the step of normalizing the expression level to a mean expression value for SLE patients with stable SLE disease.

67. The non-transitory computer readable medium of any one of claims 39-66, wherein the biomarkers are selected for inclusion in the dataset using an applied machine learning modeling approach.

68. The non-transitory computer readable medium of claim 67, wherein the applied machine learning modeling approach is one of random forest or gradient boosting.

69. The non-transitory computer readable medium of any one of claims 64-68, wherein the measurement of SLE clinical disease activity is the Safety of Estrogens in Lupus Erythematosus National Assessment-Systemic Lupus Erythematosus Disease Activity Index (SELENA-SLEDAI).

70. The non-transitory computer readable medium of any one of claims 64-69, wherein the measurement of SLE clinical disease activity is determined from samples obtained from a group of patients undergoing a flare event.

71. The non-transitory computer readable medium of any one of claims 63-70, wherein performance of the predictive model is characterized by an area under a receiver operating characteristic curve that is greater than 0.85.

72. The non-transitory computer readable medium of any one of claims 63-70, wherein performance of the predictive model is characterized by an area under a receiver operating characteristic curve that is greater than 0.90.

73. The non-transitory computer readable medium of any one of claims 39-72, wherein the future SLE disease activity event is one of a future flare event or future organ damage.

74. The non-transitory computer readable medium of any one of claims 39-73, wherein the dataset further comprises expression levels of biomarkers from a second test sample taken from the systemic lupus erythematosus (SLE) subject at a different time point.

75. A method comprising:
(a) obtaining a blood, serum, or plasma sample from the SLE subject;
(b) assessing expression levels of biomarkers from a test sample from the SLE subject, wherein the biomarkers comprise:
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin.

76. The method of claim 75, wherein
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG),
the at least two TNFR superfamily member molecules comprise tumor necrosis factor receptor I (TNFRI), tumor necrosis factor receptor II (TNFRII),
the at least two regulatory mediator molecules comprise native transforming growth factor beta (native TGF-β) and an interleukin-1 receptor antagonist (IL-1RA), and
the at least one SLE mediator molecule comprises stem cell factor (SCF).

77. The method of claim 75 or 76, wherein the biomarkers further comprise at least one T-helper type-1 (Th1) cytokines selected from interferon-gamma (IFN-γ), IL-12p70, IL-2, and IL-2R_{α}.

78. The method of claim 77, wherein the at least one Th1 cytokine comprises interferon-gamma (IFN-γ).

79. The method of claim 75, wherein:
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1);
the at least two TNFR superfamily member molecules comprise tumor necrosis factor receptor I (TNFRI), tumor necrosis factor receptor II (TNFRII), and tumor necrosis factor-related apoptosis-inducing ligand (TRAIL),
the at least two regulatory mediator molecules comprise native transforming growth factor beta (native TGF-β), an interleukin-1 receptor antagonist (IL-1RA), a total transforming growth factor beta (total TGF-β), and
the at least one SLE mediator molecule comprises stem cell factor (SCF), and
wherein the biomarkers further comprise one or more T-helper type-1 (Th1) cytokines, wherein the one or more Th1 cytokines comprise an interferon-gamma (IFN-γ).

80. A method comprising:
(a) obtaining a blood, serum, or plasma sample from the SLE subject;
(b) assessing expression levels of biomarkers from a test sample from the SLE subject, wherein the biomarkers comprise:
at least one innate cytokine selected from IL-7, IL-1α, and IL-1β;
at least one Th1 cytokine selected from interferon-gamma (IFN-γ), IL-12p70, IL-2, and IL-2R_{α};
at least one Th2 cytokine selected from IL-4 and IL-13;
at least one Th17 cytokine selected from IL-17A, IL-6, IL-21, and IL-23;
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
(b) generating a LFPI based on the expression levels in the obtained dataset; and
(c) determining likelihood of a future SLE disease activity event in the SLE subject based on the LFPI.

81. A method for assessing expression levels in a systemic lupus erythematosus (SLE) subject comprising:
(a) obtaining a blood, serum, or plasma sample from the SLE subject;
(b) assessing expression levels of biomarkers from a test sample from the SLE subject, wherein the biomarkers comprise:
chemokine(s) or adhesion molecules, wherein the chemokine(s) or adhesion molecules comprise:
a C—C motif chemokine ligand 2 (CCL2)/monocyte chemoattractant protein-1 (MCP-1),
a C—C motif chemokine ligand 3 (CCL3)/macrophage inflammatory protein-1 alpha (MIP-1α),
a C—X—C motif chemokine ligand 10 (CXCL10)/IFN-gamma-inducible protein 10 (IP-10), and
a C—X—C motif chemokine ligand 9 (CXCL9)/monokine induced by interferon-gamma (MIG);
tumor necrosis factor receptor (TNFR) superfamily member molecules, wherein the TNFR superfamily member molecules comprise:
a tumor necrosis factor receptor I (TNFRI), and
a tumor necrosis factor receptor II (TNFRII);
regulatory mediator molecules, wherein the regulatory mediator molecules comprise:
native transforming growth factor beta (native TGF-β), and
an interleukin-1 receptor antagonist (IL-1RA); and
one or more systemic lupus erythematosus (SLE) mediator molecules, wherein the one or more SLE mediator molecules comprise a stem cell factor (SCF).

82. The method of claim 81, wherein the biomarkers further comprise one or more T-helper type-1 (Th1) cytokines, wherein the one or more Th1 cytokines comprise an interferon-gamma (IFN-γ).

83. The method of claim 81 or 82, wherein the tumor necrosis factor receptor (TNFR) superfamily member molecules further comprise a tumor necrosis factor-related apoptosis-inducing ligand (TRAIL).

84. The method of any one of claims 81-83, wherein the chemokine(s) or adhesion molecules further comprise a C—C motif chemokine ligand 4 (CCL4)/macrophage inflammatory protein-1 beta (MIP-1P) and an Intercellular Adhesion Molecule 1 (ICAM-1).

85. The method of any one of claims 81-84, wherein the regulatory mediator molecules further comprise total TGF-β.

86. The method of any one of claims 81-85, wherein:
the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1);
the TNFR superfamily member molecules further comprise a tumor necrosis factor-related apoptosis-inducing ligand (TRAIL); and
the regulatory mediator molecules further comprise a total transforming growth factor beta (total TGF-β).

87. The method of any one of claims 81-86, wherein the biomarkers further comprise one or more innate cytokines, wherein the one or more innate cytokines are selected from IL-7, IL-1α, and IL-1p.

88. The method of any one of claims 81-87, wherein the biomarkers further comprise one or more T-helper type-2 (Th2) cytokines, wherein the one or more Th2 cytokines are selected from IL-13 and IL-4.

89. The method of any one of claims 81-88, wherein the biomarkers further comprise one or more Th17 cytokines, wherein the one or more Th17 cytokines comprise IL-17A.

90. The method of any one of claims 81-89, wherein the one or more SLE mediator molecules further comprise Resistin.

91. The method of any one of claims 82-90, wherein the Th1 cytokines further comprise IL-2, IL-12p70, and IL-2R_{α}.

92. The method of any one of claims 81-91, wherein the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

93. The method of any one of claims 81-92, wherein:
the Th1 cytokines comprise interferon-gamma (IFN-γ), IL-2R_{α}, IL-12p70, and IL-2;
the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8;
the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α;
the regulatory mediator molecules comprise: native transforming growth factor beta (native TGF-β), an interleukin-1 receptor antagonist (IL-1RA), a total transforming growth factor beta (total TGF-β), and IL-10;
the SLE mediator molecules comprise: a stem cell factor (SCF) and Resistin; and
wherein the biomarkers further comprise:
innate cytokines, wherein the innate cytokines comprise: IL-7, IL-1α, and IL-1β;
T-helper type-2 (Th2) cytokines, wherein the Th2 cytokines comprise: IL-13 and IL-4; and
one or more Th17 cytokines, wherein the one or more Th17 cytokines comprise IL-17A.

94. The method of any one of claims 75-93, wherein the expression levels of biomarkers comprise protein levels.

95. The method of claim 94, wherein the expression levels of biomarkers are determined using one of an ELISA assay, xMAP® technology, or SimplePlex™ assay.

96. The method of any one of claims 75-93, wherein the expression levels of biomarkers comprise mRNA levels.

97. The method of claim 96, wherein the mRNA levels are obtained from circulating cells.

98. The method of claim 96, wherein the mRNA levels are obtained from circulating T-cells.

99. The method of any one of claims 75-98, further comprising:
determining a likelihood that the SLE subject will have a future SLE disease activity event, wherein the determination comprises:
determining that expression levels of the Th1, chemokine/adhesion molecules, and TNFR superfamily member molecules are elevated and that expression levels of the regulator mediator molecules are reduced as compared to expression levels in a previous sample from the SLE subject.

100. The method of claim 99, further comprising administering a treatment to the SLE subject after determining that the SLE subject is likely to have the future SLE disease activity event.

101. The method of any one of claims 75-100, further comprising:
generating a LFPI based on the assessed expression levels.

102. The method of claim 101, wherein generating the LFPI based on the expression levels comprises applying a predictive model.

103. The method of claim 102, wherein applying the predictive model comprises,
for the assessed expression level of each biomarker:
log-transforming the expression level;
standardizing the expression level;
obtaining a corresponding coefficient for the biomarker; the corresponding coefficient representing an association between pre-flare expression levels of the biomarker and a measurement of SLE clinical disease activity; and
weighting the standardized expression level with the corresponding coefficient to obtain a LFPI subscore for the biomarker; and
summing the LFPI subscores to obtain the LFPI.

104. The method of claim 103, wherein for each assessed expression level, the corresponding coefficient is obtained from a linear regression model testing associations between the measurement of SLE clinical disease activity and the pre-flare expression levels of the biomarker.

105. The method of claim 103 or 104, wherein standardizing the assessed expression level comprises normalizing the expression level to a mean expression value for SLE patients with stable SLE disease.

106. The method of any one of claims 103-105, wherein the measurement of SLE clinical disease activity is the Safety of Estrogens in Lupus Erythematosus National Assessment-Systemic Lupus Erythematosus Disease Activity Index (SELENA-SLEDAI).

107. The method of any one of claims 103-106, wherein the measurement of SLE clinical disease activity is determined from samples obtained from a group of patients undergoing a flare event.

108. The method of any one of claims 102-107, wherein performance of the predictive model is characterized by an area under a receiver operating characteristic curve that is greater than 0.85.

109. The method of any one of claims 102-108, wherein performance of the predictive model is characterized by an area under a receiver operating characteristic curve that is greater than 0.90.

110. The method of any one of claims 99-109, wherein the future SLE disease activity event is one of a future flare event or future organ damage.

111. A computer system for assessing likelihood of a future SLE disease activity event in a systemic lupus erythematosus (SLE) subject, the computer system comprising:
a storage memory for storing a dataset comprising expression levels for biomarkers from a test sample from the SLE subject, the biomarkers comprising:
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1P), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
a processor communicatively coupled to the storage memory for determining a LFPI by applying a predictive model to the stored dataset, the LFPI predictive of the likelihood of the future SLE disease activity event in the SLE subject.

112. The computer system of claim 111, wherein:
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG),
the at least two TNFR superfamily member molecules comprise tumor necrosis factor receptor I (TNFRI), tumor necrosis factor receptor II (TNFRII),
the at least two regulatory mediator molecules comprise native transforming growth factor beta (native TGF-β) and an interleukin-1 receptor antagonist (IL-1RA), and
the at least one SLE mediator molecule comprises stem cell factor (SCF).

113. The computer system of claim 111 or 112, wherein the biomarkers further comprise at least one T-helper type-1 (Th1) cytokines selected from interferon-

114. The computer system of claim 113, wherein the at least one Th1 cytokine comprises interferon-gamma (IFN-γ).

115. The computer system of claim 111, wherein:
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1);
the at least two TNFR superfamily member molecules comprise tumor necrosis factor receptor I (TNFRI), tumor necrosis factor receptor II (TNFRII), and tumor necrosis factor-related apoptosis-inducing ligand (TRAIL),
the at least two regulatory mediator molecules comprise native transforming growth factor beta (native TGF-β), an interleukin-1 receptor antagonist (IL-1RA), a total transforming growth factor beta (total TGF-β), and
the at least one SLE mediator molecule comprises stem cell factor (SCF), and
wherein the biomarkers further comprise one or more T-helper type-1 (Th1) cytokines, wherein the one or more Th1 cytokines comprise an interferon-gamma (IFN-γ).

116. A computer system for assessing likelihood of a future SLE disease activity event in a systemic lupus erythematosus (SLE) subject, the computer system comprising:
a storage memory for storing a dataset comprising expression levels for biomarkers from a test sample from the SLE subject, the biomarkers comprising:
at least one innate cytokine selected from IL-7, IL-1α, and IL-1β;
at least one Th1 cytokine selected from interferon-gamma (IFN-γ), IL-12p70, IL-2, and IL-2R_{α};
at least one Th2 cytokine selected from IL-4 and IL-13;
at least one Th17 cytokine selected from IL-17A, IL-6, IL-21, and IL-23;
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
a processor communicatively coupled to the storage memory for determining a LFPI by applying a predictive model to the stored dataset, the LFPI predictive of the likelihood of the future SLE disease activity event in the SLE subject.

117. A computer system for assessing likelihood of a future SLE disease activity event in a systemic lupus erythematosus (SLE) subject, the computer system comprising:
a storage memory for storing a dataset comprising expression levels for biomarkers from a test sample from the SLE subject, the biomarkers comprising:
(i) chemokine(s) or adhesion molecules, wherein the chemokine(s) or adhesion molecules comprise:
a C—C motif chemokine ligand 2 (CCL2)/monocyte chemoattractant protein-1 (MCP-1),
a C—C motif chemokine ligand 3 (CCL3)/macrophage inflammatory protein-1 alpha (MIP-1α),
a C—X—C motif chemokine ligand 10 (CXCL10)/IFN-gamma-inducible protein 10 (IP-10), and
a C—X—C motif chemokine ligand 9 (CXCL9)/monokine induced by interferon-gamma (MIG);
(ii) tumor necrosis factor receptor (TNFR) superfamily member molecules, wherein the TNFR superfamily member molecules comprise:
a tumor necrosis factor receptor I (TNFRI), and
a tumor necrosis factor receptor II (TNFRII);
(iii) regulatory mediator molecules, wherein the regulatory mediator molecules comprise:
native transforming growth factor beta (native TGF-β), and
an interleukin-1 receptor antagonist (IL-1RA); and
(iv) one or more systemic lupus erythematosus (SLE) mediator molecules, wherein the one or more SLE mediator molecules comprise a stem cell factor (SCF); and
a processor communicatively coupled to the storage memory for determining a LFPI by applying a predictive model to the stored dataset, the LFPI predictive of the likelihood of the future SLE disease activity event in the SLE subject.

118. The computer system of claim 117, wherein the biomarkers further comprise one or more T-helper type-1 (Th1) cytokines, wherein the one or more Th1 cytokines comprise an interferon-gamma (IFN-γ).

119. The computer system of claim 117 or 118, wherein the tumor necrosis factor receptor (TNFR) superfamily member molecules further comprise a tumor necrosis factor-related apoptosis-inducing ligand (TRAIL).

120. The computer system of any one of claims 117-119, wherein the chemokine(s) or adhesion molecules further comprise:
a C—C motif chemokine ligand 4 (CCL4)/macrophage inflammatory protein-1 beta (MIP-1β) and an Intercellular Adhesion Molecule 1 (ICAM-1).

121. The computer system of any one of claims 117-120, wherein the regulatory mediator molecules further comprise total TGF-β.

122. The computer system of any one of claims 117-121, wherein:
the chemokine(s) or adhesion molecules further comprise a C—C motif chemokine ligand 4 (CCL4)/macrophage inflammatory protein-1 beta (MP-1β) and an Intercellular Adhesion Molecule 1 (ICAM-1);
the TNFR superfamily member molecules further comprise a tumor necrosis factor-related apoptosis-inducing ligand (TRAIL); and
the regulatory mediator molecules further comprise a total transforming growth factor beta (total TGF-β).

123. The computer system of any one of claims 117-122, wherein the biomarkers further comprise (vi) one or more innate cytokines, wherein the one or more innate cytokines are selected from IL-7, IL-1α, and IL-1β.

124. The computer system of any one of claims 117-123, wherein the biomarkers further comprise (vii) one or more T-helper type-2 (Th2) cytokines, wherein the one or more Th2 cytokines are selected from IL-13 and IL-4.

125. The computer system of any one of claims 117-124, wherein the biomarkers further comprise (viii) one or more Th17 cytokines, wherein the one or more Th17 cytokines comprise IL-17A.

126. The computer system of any one of claims 117-125, wherein the one or more SLE mediator molecules further comprise Resistin.

127. The computer system of any one of claims 118-126, wherein the Th1 cytokines further comprise IL-2, IL-12p70, and IL-2R_{α}.

128. The computer system of any one of claims 117-127, wherein the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

129. The computer system of any one of claims 117-128, wherein:
the Th1 cytokines comprise interferon-gamma (IFN-γ), IL-2R_{α}, IL-12p70, and IL-2;
the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8;
the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α;
the regulatory mediator molecules comprise: native transforming growth factor beta (native TGF-β), an interleukin-1 receptor antagonist (IL-1RA), a total transforming growth factor beta (total TGF-β), and IL-10;
the SLE mediator molecules comprise: a stem cell factor (SCF) and Resistin; and
wherein the biomarkers further comprise:
innate cytokines, wherein the innate cytokines comprise: IL-7, IL-1α, and IL-1β;
T-helper type-2 (Th2) cytokines, wherein the Th2 cytokines comprise: IL-13 and IL-4; and
one or more Th17 cytokines, wherein the one or more Th17 cytokines comprise IL-17A.

130. The computer system of any one of claims 111-129, wherein the expression levels of biomarkers comprise protein levels.

131. The computer system of claim 130, wherein the expression levels of biomarkers are determined using one of an ELISA assay, xMAP® technology, or SimplePlex™ assay.

132. The computer system of any one of claims 111-129, wherein the expression levels of biomarkers comprise mRNA levels.

133. The computer system of claim 132, wherein the mRNA levels are obtained from circulating cells.

134. The computer system of claim 132, wherein the mRNA levels are obtained from circulating T-cells.

135. The computer system of any one of claims 111-134, wherein applying the predictive model to the stored dataset comprises,
for the expression level of each biomarker:
log-transforming the expression level;
standardizing the expression level;
obtaining a corresponding coefficient for the biomarker; the corresponding coefficient representing an association between pre-flare expression levels of the biomarker and a measurement of SLE clinical disease activity; and
weighting the standardized expression level with the corresponding coefficient to obtain a LFPI subscore for the biomarker; and
summing the LFPI subscores to obtain the LFPI.

136. The computer system of claim 135, wherein for each expression level, the corresponding coefficient is obtained from a linear regression model testing associations between the measurement of SLE clinical disease activity and the pre-flare expression levels of the biomarker.

137. The computer system of claim 135 or 136, wherein standardizing the expression level comprises normalizing the expression level to a mean expression value for SLE patients with stable SLE disease.

138. The computer system of any one of claims 135-137, wherein the measurement of SLE clinical disease activity is the Safety of Estrogens in Lupus Erythematosus National Assessment-Systemic Lupus Erythematosus Disease Activity Index (SELENA-SLEDAI).

139. The computer system of any one of claims 135-138, wherein the measurement of SLE clinical disease activity is determined from samples obtained from a group of patients undergoing a flare event.

140. The computer system of any one of claims 111-139, wherein performance of the predictive model is characterized by an area under a receiver operating characteristic curve that is greater than 0.85.

141. The computer system of any one of claims 111-139, wherein performance of the predictive model is characterized by an area under a receiver operating characteristic curve that is greater than 0.90.

142. The computer system of any one of claims 111-141, wherein the future SLE disease activity event is one of a future flare event or future organ damage.

143. The computer system of any one of claims 111-142, wherein the dataset further comprises expression levels of biomarkers from a second test sample taken from the systemic lupus erythematosus (SLE) subject at a different time point.

144. A kit for assessing likelihood of a future SLE disease activity event in a systemic lupus erythematosus (SLE) subject, the kit comprising:
a set of reagents for determining expression levels for biomarkers from a test sample from the SLE subject, wherein the biomarkers comprise:
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin; and
instructions for using the set of reagents to determine the expression levels of biomarkers from the test sample.

145. The kit of claim 144, wherein
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG),
the at least two TNFR superfamily member molecules comprise tumor necrosis factor receptor I (TNFRI), tumor necrosis factor receptor II (TNFRII),
the at least two regulatory mediator molecules comprise native transforming growth factor beta (native TGF-β) and an interleukin-1 receptor antagonist (IL-1RA), and
the at least one SLE mediator molecule comprises stem cell factor (SCF).

146. The kit of claim 144 or 145, wherein the biomarkers further comprise at least one T-helper type-1 (Th1) cytokines selected from interferon-gamma (IFN-γ), IL-12p70, IL-2, and IL-2R_{α}.

147. The kit of claim 146, wherein the at least one Th1 cytokine comprises interferon-gamma (IFN-γ).

148. The kit of claim 144, wherein
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1);
the at least two TNFR superfamily member molecules comprise tumor necrosis factor receptor I (TNFRI), tumor necrosis factor receptor II (TNFRII), and tumor necrosis factor-related apoptosis-inducing ligand (TRAIL),
the at least two regulatory mediator molecules comprise native transforming growth factor beta (native TGF-β), an interleukin-1 receptor antagonist (IL-1RA), a total transforming growth factor beta (total TGF-β), and
the at least one SLE mediator molecule comprises stem cell factor (SCF), and
wherein the biomarkers further comprise one or more T-helper type-1 (Th1) cytokines, wherein the one or more Th1 cytokines comprise an interferon-gamma (IFN-γ).

149. A kit for assessing likelihood of a future SLE disease activity event in a systemic lupus erythematosus (SLE) subject, the kit comprising:
a set of reagents for determining expression levels for biomarkers from a test sample from the SLE subject, wherein the biomarkers comprise:
at least one innate cytokine selected from IL-7, IL-1α, and IL-1β;
at least one Th1 cytokine selected from interferon-gamma (IFN-γ), IL-12p70, IL-2, and IL-2R_{α};
at least one Th2 cytokine selected from IL-4 and IL-13;
at least one Th17 cytokine selected from IL-17A, IL-6, IL-21, and IL-23;
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin; and
instructions for using the set of reagents to determine the expression levels of biomarkers from the test sample.

150. A kit for assessing likelihood of a future SLE disease activity event in a systemic lupus erythematosus (SLE) subject, the kit comprising:
a set of reagents for determining expression levels for biomarkers from a test sample from the SLE subject, wherein the biomarkers comprise:
(i) chemokine(s) or adhesion molecules, wherein the chemokine(s) or adhesion molecules comprise:
a C—C motif chemokine ligand 2 (CCL2)/monocyte chemoattractant protein-1 (MCP-1),
a C—C motif chemokine ligand 3 (CCL3)/macrophage inflammatory protein-1 alpha (MIP-1α),
a C—X—C motif chemokine ligand 10 (CXCL10)/IFN-gamma-inducible protein 10 (IP-10), and
a C—X—C motif chemokine ligand 9 (CXCL9)/monokine induced by interferon-gamma (MIG);
(ii) tumor necrosis factor receptor (TNFR) superfamily member molecules, wherein the TNFR superfamily member molecules comprise:
a tumor necrosis factor receptor I (TNFRI), and
a tumor necrosis factor receptor II (TNFRII);
(iii) regulatory mediator molecules, wherein the regulatory mediator molecules comprise:
native transforming growth factor beta (native TGF-β), and
an interleukin-1 receptor antagonist (IL-1RA); and
(iv) one or more systemic lupus erythematosus (SLE) mediator molecules, wherein the one or more SLE mediator molecules comprise a stem cell factor (SCF); and
instructions for using the set of reagents to determine the expression levels of biomarkers from the test sample.

151. The kit of claim 150, wherein the biomarkers further comprise one or more T-helper type-1 (Th1) cytokines, wherein the one or more Th1 cytokines comprise an interferon-gamma (IFN-γ).

152. The kit of claim 150 or 151, wherein the tumor necrosis factor receptor (TNFR) superfamily member molecules further comprise a tumor necrosis factor-related apoptosis-inducing ligand (TRAIL).

153. The kit of any one of claims 150-152, wherein the chemokine(s) or adhesion molecules further comprise:
a C—C motif chemokine ligand 4 (CCL4)/macrophage inflammatory protein-1 beta (MIP-1β) and an Intercellular Adhesion Molecule 1 (ICAM-1).

154. The kit of any one of claims 150-153, wherein the regulatory mediator molecules further comprise total TGF-β.

155. The kit of any one of claims 150-154, wherein:
the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1);
the TNFR superfamily member molecules further comprise a tumor necrosis factor-related apoptosis-inducing ligand (TRAIL); and
the regulatory mediator molecules further comprise a total transforming growth factor beta (total TGF-β).

156. The kit of any one of claims 150-155, wherein the biomarkers further comprise (vi) one or more innate cytokines, wherein the one or more innate cytokines are selected from IL-7, IL-1α, and IL-1β.

157. The kit of any one of claims 150-156, wherein the biomarkers further comprise (vii) one or more T-helper type-2 (Th2) cytokines, wherein the one or more Th2 cytokines are selected from IL-13 and IL-4.

158. The kit of any one of claims 150-157, wherein the biomarkers further comprise (viii) one or more Th17 cytokines, wherein the one or more Th17 cytokines comprise IL-17A.

159. The kit of any one of claims 150-158, wherein the one or more SLE mediator molecules further comprise Resistin.

160. The kit of any one of claims 151-159, wherein the Th1 cytokines further comprise IL-2, IL-12p70, and IL-2R_{α}.

161. The kit of any one of claims 150-160, wherein the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

162. The kit of any one of claims 150-161, wherein:
the Th1 cytokines comprise interferon-gamma (IFN-γ), IL-2R_{α}, IL-12p70, and IL-2;
the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8;
the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α;
the regulatory mediator molecules comprise: native transforming growth factor beta (native TGF-β), an interleukin-1 receptor antagonist (IL-1RA), a total transforming growth factor beta (total TGF-β), and IL-10;
the SLE mediator molecules comprise: a stem cell factor (SCF) and Resistin; and
wherein the biomarkers further comprise:
innate cytokines, wherein the innate cytokines comprise: IL-7, IL-1α, and IL-1β;
T-helper type-2 (Th2) cytokines, wherein the Th2 cytokines comprise: IL-13 and IL-4; and
one or more Th17 cytokines, wherein the one or more Th17 cytokines comprise IL-17A.

163. The kit of any one of claims 144-162, wherein the expression levels of biomarkers comprise protein levels.

164. The kit of claim 163, wherein the expression levels of biomarkers are determined using one of an ELISA assay, xMAP® technology, or SimplePlex™ assay.

165. The kit of any one of claims 144-163, wherein the expression levels of biomarkers comprise mRNA levels.

166. The kit of claim 165, wherein the mRNA levels are obtained from circulating cells.

167. The kit of claim 165, wherein the mRNA levels are obtained from circulating T-cells.

168. The kit of any one of claims 144-167, wherein the instructions further comprise instructions for determining a LFPI from the expression levels by applying a predictive model, the LFPI predictive of the likelihood of the future SLE disease activity event in the SLE subject.

169. The kit of claim 168, wherein applying the predictive model comprises,
for the expression level of each biomarker:
log-transforming the expression level;
standardizing the expression level;
obtaining a corresponding coefficient for the biomarker; the corresponding coefficient representing an association between pre-flare expression levels of the biomarker and a measurement of SLE clinical disease activity; and
weighting the standardized expression level with the corresponding coefficient to obtain a LFPI subscore for the biomarker; and
summing the LFPI subscores to obtain the LFPI.

170. The kit of claim 169, wherein for each expression level, the corresponding coefficient is obtained from a linear regression model testing associations between the measurement of SLE clinical disease activity and the pre-flare expression levels of the biomarker.

171. The kit of claim 169 or 170, wherein standardizing the expression level comprises normalizing the expression level to a mean expression value for SLE patients with stable SLE disease.

172. The kit of any one of claims 169-171, wherein the measurement of SLE clinical disease activity is the Safety of Estrogens in Lupus Erythematosus National Assessment-Systemic Lupus Erythematosus Disease Activity Index (SELENA-SLEDAI).

173. The kit of any one of claims 169-172, wherein the measurement of SLE clinical disease activity is determined from samples obtained from a group of patients undergoing a flare event.

174. The kit of any one of claims 168-173, wherein performance of the predictive model is characterized by an area under a receiver operating characteristic curve that is greater than 0.85.

175. The kit of any one of claims 168-173, wherein performance of the predictive model is characterized by an area under a receiver operating characteristic curve that is greater than 0.90.

176. The kit of any one of claims 144-175, wherein the future SLE disease activity event is one of a future flare event or future organ damage.

177. A system for assessing likelihood of a future SLE disease activity event in a systemic lupus erythematosus (SLE) subject, the system comprising:
a set of reagents used for determining expression levels for biomarkers from a test sample from the SLE subject, wherein the biomarkers comprise:
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
an apparatus configured to receive a mixture of one or more reagents in the set and the test sample and to measure the expression levels for the biomarkers from the test sample; and
a computer system communicatively coupled to the apparatus to obtain a dataset comprising the measured expression levels for the biomarkers from the test sample and to determine a LFPI by applying a predictive model to the dataset, the LFPI predictive of the likelihood of the future SLE disease activity event in the SLE subject.

178. The system of claim 177, wherein
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG),
the at least two TNFR superfamily member molecules comprise tumor necrosis factor receptor I (TNFRI), tumor necrosis factor receptor II (TNFRII),
the at least two regulatory mediator molecules comprise native transforming growth factor beta (native TGF-β) and an interleukin-1 receptor antagonist (IL-1RA), and
the at least one SLE mediator molecule comprises stem cell factor (SCF).

179. The system of claim 177 or 178, wherein the biomarkers further comprise at least one T-helper type-1 (Th1) cytokines selected from interferon-gamma (IFN-γ), IL-12p70, IL-2, and IL-2R_{α}.

180. The system of claim 179, wherein the at least one Th1 cytokine comprises interferon-gamma (IFN-γ).

181. The system of claim 177, wherein
the at least four chemokine(s) or adhesion molecules comprise C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), and Intercellular Adhesion Molecule 1 (ICAM-1),
the at least two TNFR superfamily member molecules comprise tumor necrosis factor receptor I (TNFRI), tumor necrosis factor receptor II (TNFRII), and tumor necrosis factor-related apoptosis-inducing ligand (TRAIL),
the at least two regulatory mediator molecules comprise native transforming growth factor beta (native TGF-β), an interleukin-1 receptor antagonist (IL-1RA), a total transforming growth factor beta (total TGF-β), and
the at least one SLE mediator molecule comprises stem cell factor (SCF), and
wherein the biomarkers further comprise one or more T-helper type-1 (Th1) cytokines, wherein the one or more Th1 cytokines comprise an interferon-gamma (IFN-γ).

182. A system for assessing likelihood of a future SLE disease activity event in a systemic lupus erythematosus (SLE) subject, the system comprising:
a set of reagents used for determining expression levels for biomarkers from a test sample from the SLE subject, wherein the biomarkers comprise:
at least one innate cytokine selected from IL-7, IL-1α, and IL-1β;
at least one Th1 cytokine selected from interferon-gamma (IFN-γ), IL-12p70, IL-2, and IL-2R_{α};
at least one Th2 cytokine selected from IL-4 and IL-13;
at least one Th17 cytokine selected from IL-17A, IL-6, IL-21, and IL-23;
at least four chemokine(s) or adhesion molecules selected from C—C motif chemokine ligand 2 (CCL2)/monocyte chemoattractant protein-1 (MCP-1), C—C motif chemokine ligand 3 (CCL3)/macrophage inflammatory protein-1 alpha (MIP-1α), C—X—C motif chemokine ligand 10 (CXCL10)/IFN-gamma-inducible protein 10 (IP-10), C—X—C motif chemokine ligand 9 (CXCL9)/monokine induced by interferon-gamma (MIG), C—C motif chemokine ligand 4 (CCL4)/macrophage inflammatory protein-1 beta (MIP-1β), Intercellular Adhesion Molecule 1 (ICAM-1), CCL7/MCP-3, VCAM-1, and CXCL8/IL-8;
at least two TNFR superfamily member molecules selected from tumor necrosis factor receptor I (TNFRI), tumor necrosis factor receptor II (TNFRII), tumor necrosis factor-related apoptosis-inducing ligand (TRAIL), Fas, NGF-β, and TNF-α;
at least two regulatory mediator molecules selected from native transforming growth factor beta (native TGF-β), an interleukin-1 receptor antagonist (IL-1RA), a total transforming growth factor beta (total TGF-β), and IL-10; and
at least one SLE mediator molecule selected from a stem cell factor (SCF) and Resistin;
an apparatus configured to receive a mixture of one or more reagents in the set and the test sample and to measure the expression levels for the biomarkers from the test sample; and
a computer system communicatively coupled to the apparatus to obtain a dataset comprising the measured expression levels for the biomarkers from the test sample and to determine a LFPI by applying a predictive model to the dataset, the LFPI predictive of the likelihood of the future SLE disease activity event in the SLE subject.

183. A system for assessing likelihood of a future SLE disease activity event in a systemic lupus erythematosus (SLE) subject, the system comprising:
a set of reagents used for determining expression levels for biomarkers from a test sample from the SLE subject, wherein the biomarkers comprise:
(i) chemokine(s) or adhesion molecules, wherein the chemokine(s) or adhesion molecules comprise:
a C—C motif chemokine ligand 2 (CCL2)/monocyte chemoattractant protein-1 (MCP-1),
a C—C motif chemokine ligand 3 (CCL3)/macrophage inflammatory protein-1 alpha (MIP-1α),
a C—X—C motif chemokine ligand 10 (CXCL10)/IFN-gamma-inducible protein 10 (IP-10), and
a C—X—C motif chemokine ligand 9 (CXCL9)/monokine induced by interferon-gamma (MIG);
(ii) tumor necrosis factor receptor (TNFR) superfamily member molecules, wherein the TNFR superfamily member molecules comprise:
a tumor necrosis factor receptor I (TNFRI), and
a tumor necrosis factor receptor II (TNFRII);
(iii) regulatory mediator molecules, wherein the regulatory mediator molecules comprise:
native transforming growth factor beta (native TGF-β), and
an interleukin-1 receptor antagonist (IL-1RA); and
(iv) one or more systemic lupus erythematosus (SLE) mediator molecules, wherein the one or more SLE mediator molecules comprise a stem cell factor (SCF);
an apparatus configured to receive a mixture of one or more reagents in the set and the test sample and to measure the expression levels for the biomarkers from the test sample; and
a computer system communicatively coupled to the apparatus to obtain a dataset comprising the measured expression levels for the biomarkers from the test sample and to determine a LFPI by applying a predictive model to the dataset, the LFPI predictive of the likelihood of the future SLE disease activity event in the SLE subject.

184. The system of claim 183, wherein the biomarkers further comprise (i) one or more T-helper type-1 (Th1) cytokines, wherein the one or more Th1 cytokines comprise an interferon-gamma (IFN-γ).

185. The system of claim 183 or 184, wherein the tumor necrosis factor receptor (TNFR) superfamily member molecules further comprise a tumor necrosis factor-related apoptosis-inducing ligand (TRAIL).

186. The system of any one of claims 183-185, wherein the chemokine(s) or adhesion molecules further comprise:
a C—C motif chemokine ligand 4 (CCL4)/macrophage inflammatory protein-1 beta (MIP-1β) and an Intercellular Adhesion Molecule 1 (ICAM-1).

187. The system of any one of claims 183-186, wherein the regulatory mediator molecules further comprise total TGF-β.

188. The system of any one of claims 183-187, wherein:
the chemokine(s) or adhesion molecules further comprise a C—C motif chemokine ligand 4 (CCL4)/macrophage inflammatory protein-1 beta (MIP-1β) and an Intercellular Adhesion Molecule 1 (ICAM-1);
the TNFR superfamily member molecules further comprise a tumor necrosis factor-related apoptosis-inducing ligand (TRAIL); and
the regulatory mediator molecules further comprise a total transforming growth factor beta (total TGF-β).

189. The system of any one of claims 183-188, wherein the biomarkers further comprise (vi) one or more innate cytokines, wherein the one or more innate cytokines are selected from IL-γ, IL-1α, and IL-1β.

190. The system of any one of claims 183-189, wherein the biomarkers further comprise (vii) one or more T-helper type-2 (Th2) cytokines, wherein the one or more Th2 cytokines are selected from IL-13 and IL-4.

191. The system of any one of claims 183-190, wherein the biomarkers further comprise (viii) one or more Th17 cytokines, wherein the one or more Th17 cytokines comprise IL-17A.

192. The system of any one of claims 183-191, wherein the one or more SLE mediator molecules further comprise Resistin.

193. The system of any one of claims 184-192, wherein the Th1 cytokines further comprise IL-2, IL-12p70, and IL-2R_{α}.

194. The system of any one of claims 183-193, wherein the chemokine(s) or adhesion molecules further comprise CCL7/MCP-3, VCAM-1, and CXCL8/IL-8, wherein the tumor necrosis factor receptor (TNFR) superfamily member molecules further comprise Fas, NGF-β, and TNF-α, and wherein the regulatory mediator molecules further comprise IL-10.

195. The system of any one of claims 183-194, wherein:
the Th1 cytokines comprise interferon-gamma (IFN-γ), IL-2R_{α}, IL-12p70, and IL-2;
the chemokine(s) or adhesion molecules comprise: a C—C motif chemokine ligand 2 (CCL2)/monocyte chemoattractant protein-1 (MCP-1), a C—C motif chemokine ligand 3 (CCL3)/macrophage inflammatory protein-1 alpha (MIP-1α), a C—X—C motif chemokine ligand 10 (CXCL10)/IFN-gamma-inducible protein 10 (IP-10), a C—X—C motif chemokine ligand 9 (CXCL9)/monokine induced by interferon-gamma (MIG), a C—C motif chemokine ligand 4 (CCL4)/macrophage inflammatory protein-1 beta (MIP-1β), an Intercellular Adhesion Molecule 1 (ICAM-1), CCL7/MCP-3, VCAM-1, and CXCL8/IL-8;
the TNFR superfamily member molecules comprise: a tumor necrosis factor receptor I (TNFRI), a tumor necrosis factor receptor II (TNFRII), a tumor necrosis factor-related apoptosis-inducing ligand (TRAIL), Fas, NGF-β, and TNF-α;
the regulatory mediator molecules comprise: native transforming growth factor beta (native TGF-β), an interleukin-1 receptor antagonist (IL-1RA), a total transforming growth factor beta (total TGF-β), and IL-10;
the SLE mediator molecules comprise: a stem cell factor (SCF) and Resistin; and
wherein the biomarkers further comprise:
innate cytokines, wherein the innate cytokines comprise: IL-7, IL-1α, and IL-1β;
T-helper type-2 (Th2) cytokines, wherein the Th2 cytokines comprise: IL-13 and IL-4; and
one or more Th17 cytokines, wherein the one or more Th17 cytokines comprise IL-17A.

196. The system of any one of claims 177-195, wherein the expression levels comprise protein levels.

197. The system of claim 196, wherein the expression levels of biomarkers are determined using one of an ELISA assay, xMAP® technology, or SimplePlex™ assay.

198. The system of any one of claims 177-196, wherein the expression levels comprise mRNA levels.

199. The system of claim 198, wherein the mRNA levels are obtained from circulating cells.

200. The system of claim 198, wherein the mRNA levels are obtained from circulating T-cells.

201. The system of any one of claims 177-200, wherein applying the predictive model to the dataset comprises,
for the expression level of each biomarker:
log-transforming the expression level;
standardizing the expression level;
obtaining a corresponding coefficient for the biomarker; the corresponding coefficient representing an association between pre-flare expression levels of the biomarker and a measurement of SLE clinical disease activity; and
weighting the standardized expression level with the corresponding coefficient to obtain a LFPI subscore for the biomarker; and
summing the LFPI subscores to obtain the LFPI.

202. The system of claim 201, wherein for each expression level, the corresponding coefficient is obtained from a linear regression model testing associations between the measurement of SLE clinical disease activity and the pre-flare expression levels of the biomarker.

203. The system of claim 201 or 202, wherein standardizing the expression level comprises normalizing the expression level to a mean expression value for SLE patients with stable SLE disease.

204. The system of any one of claims 201-203, wherein the measurement of SLE clinical disease activity is the Safety of Estrogens in Lupus Erythematosus National Assessment-Systemic Lupus Erythematosus Disease Activity Index (SELENA-SLEDAI).

205. The system of any one of claims 201-204, wherein the measurement of SLE clinical disease activity is determined from samples obtained from a group of patients undergoing a flare event.

206. The system of any one of claims 177-205, wherein performance of the predictive model is characterized by an area under a receiver operating characteristic curve that is greater than 0.85.

207. The system of any one of claims 177-205, wherein performance of the predictive model is characterized by an area under a receiver operating characteristic curve that is greater than 0.90.

208. The system of any one of claims 177-205, wherein performance of the predictive model is characterized by an area under a receiver operating characteristic curve that is greater than 0.94.

209. The system of any one of claims 177-208, wherein the future SLE disease activity event is one of a future flare event or future organ damage.
